(19) United States
(12) Patent Application Publication
(10) Pub. No.: US 2021/0102007 A1
(43) Pub. Date: Apr. 8, 2021

(54) REFINED BETA-GLUCAN AND METHODS OF MAKING THE SAME

(71) Applicant: CARGILL, INCORPORATED, Wayzata, MN (US)

(72) Inventors: Dominique LELIMOUSIN, Cretteville (FR); Jeffrey J. MALSAM, Minneapolis, MN (US); Eric Stanley SUMNER, Minneapolis, MN (US)

(73) Assignee: CARGILL, INCORPORATED, Wayzata, MN (US)

(21) Appl. No.: 16/499,733

(22) PCT Filed: Mar. 23, 2018
(86) PCT No.: PCT/US2018/024039

§ 371 Date: Sep. 30, 2019

Related U.S. Application Data
(60) Provisional application No. 62/477,646, filed on Mar. 28, 2017.

(51) Int. Cl. C08B 37/00; C09K 8/588

(52) U.S. Cl. CPC C08B 37/0024; C09K 8/588

(57) ABSTRACT

Refined beta-glucans, such as scleroglucan or schizophyllan, and methods of making and using the same, such as for treating subterranean formations. A method of making a refined beta-glucan includes filtering a solution of a crude beta-glucan.

### CROSS-REFERENCE TO RELATED APPLICATION

This application claims the benefit of priority to U.S. Provisional Patent Application Ser. No. 62/477,646 filed Mar. 28, 2017, the disclosure of which is incorporated herein in its entirety by reference.

### BACKGROUND

Beta-glucans can be used as thickeners in aqueous fluids for treatment of subterranean formations, such as for enhanced oil recovery (EOR). Due to transportation costs and lack of space (particularly for off-shore applications), a fully-diluted and ready-to-use aqueous beta-glucan solution is expensive and undesirable; therefore, a solid or concentrated form of the beta-glucan is preferable for such applications to avoid the unneeded transport of water. However, conventional forms of beta-glucans are difficult to solubilize or disperse into solution to form effective subterranean treatment fluids and suffer from problems such as long required mixing times, high shear requirements for mixing, insufficient viscosity build during mixing, and poor filterability during subterranean use (e.g., clogs pores of subterranean formations). In addition, controlling viscosity and filterability (e.g., resistance to clogging of subterranean formation pores) of aqueous compositions including conventional forms of beta-glucans can be difficult and inconvenient, with compositions having acceptable filterabilities often having lower than desired viscosities.

### SUMMARY OF THE INVENTION

In various aspects, the present invention provides a refined beta-glucan. The refined beta-glucan can be characterized by any one or any combination of features described herein.

Various aspects of the present invention provide a refined beta-glucan. A dispersed mixture of the beta-glucan in water at a concentration of 1 mg/mL has an obscuration of less than or equal to about 0.7%. The T_{g} of the beta-glucan as measured by onset of storage modulus change as detected by dynamic mechanical analysis is about 50° C. to about 90° C. The T_{g} of the beta-glucan as measured by the peak tan delta as detected by dynamic mechanical analysis is about 70° C. to about 110° C. The beta-glucan has a majority decomposition temperature of about 300° C. to about 350° C. About 80 wt % to about 98 wt % of the beta-glucan is dry matter. The beta-glucan has a total atomic calcium content of about 300 μg/g to about 10,000 μg/g. The beta-glucan has a total atomic copper content of about 0 μg/g to about 4 μg/g. The beta-glucan has a total atomic iron content of about 10 μg/g to about 300 μg/g. A total atomic potassium content of about 0 μg/g to about 500 μg/g. The beta-glucan has a total atomic magnesium content of about 1 μg/g to about 14,000 μg/g. The beta-glucan has a total atomic manganese content of about 0.1 μg/g, to about 30 μg/g. The beta-glucan has a total atomic sodium content of about 100 μg/g to about 4,000 μg/g. The beta-glucan has a total atomic phosphorus content of about 0 μg/g to about 15,000 μg/g. The beta-glucan has a total atomic sulfur content of about 50 μg/g to about 400 μg/g. The beta-glucan has a total atomic zinc content of about 0 μg/g to about 15 μg/g. The beta-glucan has a total atomic nitrogen content of about 1 μg/g to about 10 μg/g.

Various aspects of the present invention provide a refined beta-glucan. A dispersed mixture of the beta-glucan in water at a concentration of 1 mg/mL has an obscuration of about 0.01% to about 0.6%. The T_{g} of the beta-glucan as measured by onset of storage modulus change as detected by dynamic mechanical analysis is about 60° C. to about 80° C., The T_{g} of the beta-glucan as measured by the peak tan delta as detected by dynamic mechanical analysis is about 85° C. to about 100° C., The beta-glucan has a majority decomposition temperature of about 315° C. to about 340° C. About 80 wt % to about 98 wt % of the beta-glucan is dry matter. The beta-glucan has a total atomic calcium content of about 500 μg/g to about 9,000 μg/g. The beta-glucan has a total atomic copper content of about 0 μg/g to about 3 μg/g. The beta-glucan has a total atomic iron content of about 40 μg/g to about 290 μg/g. The beta-glucan has a total atomic potassium content of about 0 μg/g to about 300 μg/g. The beta-glucan has a total atomic magnesium content of about 5 μg/g to about 13,000 μg/g. The beta-glucan has a total atomic manganese content of about 1 μg/g to about 20 μg/g. The beta-glucan has a total atomic sodium content of about 200 μg/g to about 3,200 μg/g. The beta-glucan has a total atomic phosphorus content of about 0 μg/g to about 12,000 μg/g. The beta-glucan has a total atomic sulfur content of about 100 μg/g to about 350 μg/g. The beta-glucan has a total atomic zinc content of about 0 μg/g to about 13 μg/g. The beta-glucan has a total atomic nitrogen content of about 2 μg/g to about 7 μg/g.

Various aspects of the present invention provide a refined beta-glucan. The beta-glucan is scleroglucan. A dispersed mixture of the beta-glucan in water at a concentration of 1 mg/mL, has an obscuration of about 0.001% to about 0.5%. The T_{g} of the beta-glucan as measured by the onset of storage modulus change as detected by dynamic mechanical analysis is about 70° C. to about 80° C. The T_{g} of the beta-glucan as measured by the peak tan delta as detected by dynamic mechanical analysis is about 90° C. to about 105° C. The beta-glucan has a majority decomposition temperature of about 330° C. to about 350° C. About 80 wt % to about 98 wt % of the beta-glucan is dry matter. The beta-glucan has a total atomic calcium content of about 300 μg/g to about 4,500 μg/g. The beta-glucan has a total atomic copper content of about 0 μg/g to about 4 μg/g. The beta-glucan has a total atomic iron content of about 150 μg/g to about 300 μg/g. The beta-glucan has a total atomic potassium content of about 0 μg/g, to about 200 μg/g. The beta-glucan has a total atomic magnesium content of about 1 μg/g to about 100 μg/g. The beta-glucan has a total atomic manganese content of about 0.2 μg/g to about 2 μg/g. The beta-glucan has a total atomic sodium content of about 100 μg/g to about 3,500 μg/g. The beta-glucan has a total atomic phosphorus content of about 0 μg/g to about 500 μg/g. The beta-glucan has a total atomic sulfur content of about 50 μg/g to about 300 μg/g. The beta-glucan has a total atomic zinc content of about 0 μg/g to about 4 μg/g. The beta-glucan has a total atomic nitrogen content of about 1 μg/g to about 5 μg/g. Upon total combustion the beta-glucan forms an ash that is about 0.1 wt % to about 1.3 wt % of the beta-glucan.

Various aspects of the present invention provide a refined beta-glucan. The beta-glucan is scleroglucan. Protein is about 0.10 wt % to about 0.20 wt % of the beta-glucan. A dispersed mixture of the beta-glucan in water at a concentration of 1 mg/mL has an obscuration of about 0.01% to about 0.35%. The T_{g} of the beta-glucan as measured by the onset of storage modulus change as detected by dynamic mechanical analysis is about 72° C. to about 76° C. The T_{g} of the beta-glucan as measured by the peak tan delta as detected by dynamic mechanical analysis is about 97° C. to about 99° C. The beta-glucan has a majority decomposition temperature of about 335° C. to about 345° C. About 80 wt % to about 98 wt % of the beta-glucan is dry matter. The beta-glucan has a total atomic calcium content of about 500 μg/g to about 4,100 μg/g. The beta-glucan has a total atomic copper content of about 0 μg/g to about 3.5 μg/g. The beta-glucan has a total atomic iron content of about 160 μg/g to about 290 μg/g. The beta-glucan has a total atomic potassium content of about 0 μg/g to about 125 μg/g. The beta-glucan has a total atomic magnesium content of about 5 μg/g to about 50 μg/g. The beta-glucan has a total atomic manganese content of about 0.1 μg/g to about 1.9 μg/g, The beta-glucan has a total atomic sodium content of about 250 μg/g to about 3,200 μg/g. The beta-glucan has a total atomic phosphorus content of about 0 μg/g to about 300 μg/g. The beta-glucan has a total atomic sulfur content of about 100 μg/g to about 250 μg/g. The beta-glucan has a total atomic zinc content of about 0 μg/g to about 3 μg/g. The beta-glucan has a total atomic nitrogen content of about 2.5 μg/g to about 3 μg/g. Upon total combustion the beta-glucan forms an ash that is about 0.1 wt % to about 1.2 wt % of the beta-glucan.

Various aspects of the present invention provide a refined beta-glucan. The beta-glucan is schizophyllan. A dispersed mixture of the beta-glucan in water at a concentration of 1 mg/mL has an obscuration of about 0.3% to about 0.7%. The T_{g} of the beta-glucan as measured by onset of storage modulus change as detected by dynamic mechanical analysis is about 60° C. to about 70° C. The T_{g} of the beta-glucan as measured by the peak tan delta as detected by dynamic mechanical analysis is about 85° C. to about 95° C. The beta-glucan has a majority decomposition temperature of about 340° C. to about 355° C. About 80 wt % to about 98 wt % of the beta-glucan is dry matter. The beta-glucan has a total atomic calcium content of about 7,000 μg/g to about 10,000 μg/g. The beta-glucan has a total atomic copper content of about 0.5 μg/g to about 2 μg/g. The beta-glucan has a total atomic iron content of about 30 μg/g to about 80 μg/g. The beta-glucan has a total atomic potassium content of about 250 μg/g to about 310 μg/g. The beta-glucan has a total atomic magnesium content of about 12,000 μg/g to about 14,000 μg/g. The beta-glucan has a total atomic manganese content of about 14 μg/g to about 25 μg/g. The beta-glucan has a total atomic sodium content of about 150 μg/g to about 350 μg/g. The beta-glucan has a total atomic phosphorus content of about 10,000 μg/g to about 12,000 μg/g. The beta-glucan has a total atomic sulfur content of about 200 μg/g to about 400 μg/g. The beta-glucan has a total atomic zinc content of about 10 μg/g to about 16 μg/g. The beta-glucan has a total atomic nitrogen content of about 4 μg/g to about 8 μg/g.

Various aspects of the present invention provide a refined beta-glucan. The beta-glucan is schizophyllan. Protein is about 0.35 wt % to about 0.45 wt % of the beta-glucan. A dispersed mixture of the beta-glucan in water at a concentration of 1 mg/mL has an obscuration of about 0.4% to about 0.5%. The T_{g} of the beta-glucan as measured by onset of storage modulus change as detected by dynamic mechanical analysis is about 65° C. to about 66° C. The T_{g} of the beta-glucan as measured by the peak tan delta as detected by dynamic mechanical analysis is about 89° C. to about 90° C. The beta-glucan has a majority decomposition temperature of about 345° C. to about 350° C. About 80 wt % to about 98 wt % of the beta-glucan is dry matter. The beta-glucan has a total atomic calcium content of about 8,000 μg/g to about 9,000 μg/g. The beta-glucan has a total atomic copper content of about 1.1 μg/g to about 1.5 μg/g. The beta-glucan has a total atomic iron content of about 45 μg/g to about 60 μg/g. The beta-glucan has a total atomic potassium content of about 260 μg/g to about 300 μg/g. The beta-glucan has a total atomic magnesium content of about 12,800 μg/g to about 12,900 μg/g. The beta-glucan has a total atomic manganese content of about 16 μg/g to about 22 μg/g. The beta-glucan has a total atomic sodium content of about 200 μg/g to about 300 μg/g. The beta-glucan has a total atomic phosphorus content of about 10,500 μg/g to about 11,500 μg/g. The beta-glucan has a total atomic sulfur content of about 250 μg/g to about 350 μg/g. The beta-glucan has a total atomic zinc content of about 12 μg/g to about 14 μg/g. The beta-glucan has a total atomic nitrogen content of about 5.5 μg/g to about 6.5 μg/g.

Various aspects of the present invention provide a composition including the refined beta-glucan. The composition can be a liquid, a solid, or a combination thereof (e.g., a suspension).

Various aspects of the present invention provide a method of forming a beta-glucan. The method includes filtering a solution of a crude beta-glucan, to form a filtrate that includes the beta-glucan. The beta-glucan can be precipitated from the filtrate, to provide the refined beta-glucan described herein. Various aspects of the present invention provide a refined beta-glucan that is made by the method.

Various aspects of the present invention provide a method of forming a refined beta-glucan. The method includes filtering a solution of a crude beta-glucan, including adding one or more filter aids to the solution and filtering all or a portion of the solution through a filter to form a filter cake on a filter, and filtering all of the solution through the filter cake on the filter, to form a first filtrate. The method includes filtering the first filtrate, including adding one or more filter aids to the solution and filtering all or a portion of the solution through a filter to form a filter cake on a filter, and filtering all of the solution through the filter cake on the filter, to form a second filtrate. The method includes filtering the second filtrate, including adding one or more filter aids to the solution and filtering all or a portion of the solution through a filter to form a filter cake on a filter, and filtering all of the solution through the filter cake on the filter, to form a third filtrate. The method includes precipitating biopolymer from the third filtrate, including adding an organic solvent to the filtrate to decrease the solubility of the biopolymer therein, and draining liquid from the precipitated biopolymer. The method includes washing the biopolymer with an organic solvent and draining the organic solvent wash from the biopolymer. The method includes drying the biopolymer such that the biopolymer has a dry matter content of about 80 wt % to about 98 wt %. The method includes grinding the dried biopolymer to a particle size of equal to or less than about 1,000 microns, to provide the refined beta-glucan. Each filtration is independently performed at a temperature of about 40° C. to about 90° C. The concentration of each filter aid is independently about 1 g/L to about 100 g/L. Each filter aid independently has a permeability of about 0.001 Darcy to about 30 Darcy.

Various aspects of the present invention provide a method of treating a subterranean formation. The method includes placing the refined beta-glucan in the subterranean formation. In some aspects, the method includes enhanced oil recovery, hydraulic fracturing, water shut-off, conformance, or a combination thereof.

Various aspects of the present invention have advantages over other beta-glucans and methods of making and using the same, at least some of which are unexpected, For example, some beta-glucans can require long mixing times, high shear rates, or a combination thereof, to disperse the beta-glucan in water. In various aspects, the refined beta-glucan of the present invention, in a dry or concentrated liquid state (e.g., a suspension or a solution), can more easily be combined with aqueous liquids to form homogeneous solutions than other beta-glucans. In various aspects, the refined beta-glucan of the present invention can provide a homogeneous mixture of water and the beta-glucan using a shorter mixing time, less shear, or a combination thereof, as compared to other beta-glucans.

With conventional beta-glucans it can be difficult or impossible to prepare fully-diluted and ready-to-use aqueous solutions using salt water, especially with high salt concentrations, due to problems such as insufficient viscosity and insufficient dispersion of the beta-glucan in the water. In various aspects, the refined beta-glucan of the present invention can be diluted using salt water to form a homogenous mixture of the water and the beta-glucan with better dispersion of the beta-glucan (e.g., more dispersed), less mixing time or lower shear rate for preparation, better viscosity performance (e.g., faster viscosity build or higher final viscosity), or a combination thereof, as compared to other beta-glucans.

Conventional beta-glucans can suffer from slow or insufficient viscosity build during mixing with water, such that an ultimate viscosity of the fully-diluted and dispersed beta-glucan can only be achieved with long mixing times or can never be achieved. In various aspects, a solution including the refined beta-glucan of the present invention can build viscosity faster (e.g., can reach maximum viscosity more quickly and easily) than solutions made with existing commercially available beta-glucan materials. Some beta-glucans can form fully-diluted and ready-to-use treatment fluids that perform poorly under heated conditions (e.g., 70° C. to 150° C.), such as having insufficient or decreasing viscosity. In various aspects, the refined beta-glucan of the present invention can be used to form a homogenous mixture of the water and the beta-glucan with better performance under heated conditions, such as higher viscosity or less or no viscosity degradation, as compared to other beta-glucans.

Some subterranean formation treatment fluids can clog pores and flowpaths in subterranean formations which can result in decreased production rates or increased pressures that can damage the formation. In various aspects, a solution including the refined beta-glucan of the present invention can provide higher filterability (e.g., lower Filterability Ratio) than solutions made with other beta-glucans.

In various aspects, a solution including the refined beta-glucan of the present invention can maintain viscosity more effectively during various filtration procedures, such as various procedures for treatment of a subterranean formation, as compared to solutions formed with other beta-glucans. In various aspects, a solution of the refined beta-glucan of the present invention used for treatment of a subterranean formation can have a lower injection pressure at the same viscosity and the same injection rate (e.g., at the same injection pressure a higher injection rate can occur), as compared to solutions formed with other viscosifiers such as other beta-glucans.

In various aspects, the refined beta-glucan of the present invention can have increased thermal stability, as indicated by higher T_{g} values, than other beta-glucans, allowing higher maximum reservoir temperatures for treatment of subterranean formations, such as for enhanced oil recovery. In various aspects, the refined beta-glucan of the present invention can resist or avoid forming solid precipitants in the presence of high levels of Ca²⁺ and Mg²⁺ ions to a greater extent than other viscosifying materials.

In various aspects a low impurity level of the refined beta-glucan of the present invention can reduce mineral and nutrient loading a solution formed therefrom, as compared to solutions formed from other beta-glucans. In various aspects, filtration of a solution of the refined beta-glucan of the present invention prior to injection into a subterranean formation can be conducted with less loading of the filter (e.g., less accumulation on the filter per time), with a need for less cleaning or replacement of filters as compared to solutions formed with other viscosifiers such as other beta-glucans.

In various aspects, the particle size distribution of the refined beta-glucan of the present invention can provide good flow characteristics for transport (e.g., can be a narrow distribution to facilitate flow characteristics), can be large enough to avoid explosion risks or dust health hazards, and can be small enough to accelerate solubilization.

### BRIEF DESCRIPTION OF THE FIGURES

The drawings illustrate generally, by way of example, but not by way of limitation, various aspects of the present invention.

FIG. 1A illustrates a top view of a stirrer, in accordance with various aspects.

FIG. 1B illustrates a side view of the bend of the stirrer, as viewed perpendicularly to one the slot adjacent to the bend.

FIG. 2A illustrates storage modulus versus temperature for various beta-glucan compositions, according to various aspects.

FIG. 2B illustrates tan delta versus temperature for various beta-glucan compositions, according to various aspects.

FIGS. 3A-3H illustrate atomic-force microscopy images of a beta-glucan composition at 2 micron and 10 micron image size, in accordance with various aspects.

FIGS. 4A-4I illustrate atomic-force microscopy images of a beta-glucan composition at 2 micron and 10 micron image size, in accordance with various aspects.

FIGS. 5A-5H illustrate atomic-force microscopy images of a beta-glucan composition at 2 micron and 10 micron image size, in accordance with various aspects.

FIGS. 6A-6I illustrate atomic-force microscopy images of a beta-glucan composition at 2 micron and 10 micron image size, in accordance with various aspects.

FIGS. 7A-7J illustrate atomic-force microscopy images of a beta-glucan composition at 2 micron and 10 micron image size, in accordance with various aspects.

FIGS. 8A-8H illustrate confocal laser scanning microscopy images of various beta-glucan compositions with staining to illustrate carbohydrates or protein, in accordance with various aspects.

FIGS. 9A-9F illustrate confocal laser scanning microscopy images of a beta-glucan composition with staining to illustrate carbohydrates or protein, in accordance with various aspects.

FIGS. 10A-10E illustrate confocal laser scanning microscopy images of a beta-glucan composition with staining to illustrate carbohydrates or protein, in accordance with various aspects.

FIGS. 11A-11D illustrate confocal laser scanning microscopy images of a beta-glucan composition with staining to illustrate carbohydrates or protein, in accordance with various aspects.

FIGS. 12A-12F illustrate confocal laser scanning microscopy images of a beta-glucan composition with staining to illustrate carbohydrates or protein, in accordance with various aspects.

FIG. 13A illustrates weight % versus temperature for various beta-glucan compositions during thermogravimetric analysis, in accordance with various aspects.

FIG. 13B illustrates weight % versus temperature for various beta-glucan compositions during thermogravimetric analysis, in accordance with various aspects.

FIG. 14 illustrates Viscosity Build versus passes through the Magic Lab for various beta-glucan compositions, in accordance with various aspects.

FIG. 15 illustrates mass of filtrate versus time for various solubilized beta-glucan compositions, in accordance with various aspects.

FIG. 16 illustrates heat flow versus temperature for various beta-glucan compositions, in accordance with various aspects.

FIG. 17A illustrates flowrate and delta P versus time in a permeability measurement of a sand pack column, in accordance with various embodiments.

FIG. 17B illustrates delta P and permeability versus flowrate in a permeability measurement of a sand pack column, in accordance with various embodiments.

FIG. 18 illustrates the pressure drop versus the total flow for a sand pack test for various beta-glucan compositions, in accordance with various aspects.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99% 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0,2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “standard temperature and pressure” as used herein refers to 20° C. and 101 kPa.

The term “downhole” as used herein refers to under the surface of the earth, such as a location within or fluidly connected to a wellbore.

As used herein, the term “subterranean material” or “subterranean formation” refers to any material under the surface of the earth, including under the surface of the bottom of the ocean. For example, a subterranean formation or material can be any section of a wellbore and any section of a subterranean petroleum- or water-producing formation or region in fluid contact with the wellbore. Placing a material in a subterranean formation can include contacting the material with any section of a wellbore or with any subterranean region in fluid contact therewith. Subterranean materials can include any materials placed into the wellbore such as cement, drill shafts, liners, tubing, casing, or screens; placing a material in a subterranean formation can include contacting with such subterranean materials. In some examples, a subterranean formation or material can be any below-ground region that can produce liquid or gaseous petroleum materials, water, or any section below-ground in fluid contact therewith. For example, a subterranean formation or material can be at least one of an area desired to be fractured, a fracture or an area surrounding a fracture, and a flow pathway or an area surrounding a flow pathway, wherein a fracture or a flow pathway can be optionally fluidly connected to a subterranean petroleum- or water-producing region, directly or through one or more fractures or flow pathways.

As used herein, “treatment of a subterranean formation” can include any activity directed to extraction of water or petroleum materials from a subterranean petroleum- or water-producing formation or region, for example, including drilling, stimulation, hydraulic fracturing, clean-up, acidizing, completion, cementing, remedial treatment, abandonment, water shut-off, conformance, and the like.

As used herein, a “flow pathway” downhole can include any suitable subterranean flow pathway through which two subterranean locations are in fluid connection. The flow pathway can be sufficient for petroleum or water to flow from one subterranean location to the wellbore or vice-versa. A flow pathway can include at least one of a hydraulic fracture, and a fluid connection across a screen, across gravel pack, across proppant, including across resin-bonded proppant or proppant deposited in a fracture, and across sand. A flow pathway can include a natural subterranean passageway through which fluids can flow. In some aspects, a flow pathway can be a water source and can include water. In some aspects, a flow pathway can be a petroleum source and can include petroleum. In some aspects, a flow pathway can be sufficient to divert from a wellbore, fracture, or flow pathway connected thereto at least one of water, a downhole fluid, or a produced hydrocarbon.

### Refined Beta-Glucan.

Various aspects of the present invention provide a refined beta-glucan. The beta-glucan is refined (e.g., isolated, separated, or purified) from a crude beta-glucan, such as from fermentation broth including microorganisms that generated the beta-glucan or such as from any suitable commercially available beta-glucan material, such as Cargill's Actigum® CS-6 or CS-11 materials. The refined beta-glucan can be free of other materials, such as free of a fermentation broth and associated contaminants therein. For example, the refined beta-glucan can have a purity of at least 75 wt % (e.g., less than 25 wt % contaminates), at least 80 wt %, about 75 to about 100 wt %, about 80 to about 95 wt %, about 82 to about 92 wt %, or greater than, equal to, or less than about 75 wt %, 76, 78, 80, 81, 82, 83, 84, 85, 86, 87, 88, 89, 90, 91, 92, 93, 94, 95, 96, 97, 98, 99, 99.9, or about 99.99 wt % or more purity. The refined beta-glucan can be characterized in various ways, such as by any one or combination of characterization features described herein, by the method of making the refined beta-glucan described herein, or a combination thereof. The refined beta-glucan can be a solid, such as a powder (e.g., dry or sticky), fibers, a monolithic solid, or a combination thereof. The refined beta-glucan can be part of a composition such as a liquid or solid composition, wherein the composition is different than the crude beta-glucan or the fermentation broth that formed the same.

The beta-glucan can be a 1,3 beta-glucan. The beta-glucan can be a 1,3-1,4 beta-D-glucan. The beta-glucan can be a 1,3-1,6 beta-D-glucan, such as having a main chain from beta-1,3-glycosidically bonded glucose units, and side groups which are formed from glucose units and are beta-1,6-glycosidically bonded thereto. Examples of 1,3 beta-D-glucans include curdlan (a homopolymer of beta-(1,3)-linked D-glucose residues produced from, e.g., Agrobacterium spp.), grifolan (a branched beta-(1,3)-D-glucan produced from, e.g., the fungus Grifola frondosa), lentinan (a branched beta-(1,3)-D-glucan having two glucose branches attached at each fifth glucose residue of the beta-(1,3)-backbone produces from, e.g., the fungus Lentinus eeodes), schizophyllan (a branched beta-(1,3)-D-glucan having one glucose branch for every third glucose residue in the beta-(1,3)-backbone produced from, e.g., the fungus Schizophyllan commune), scleroglucan (a branched beta-(1,3)-D-glucan with one out of three glucose molecules of the beta-(1,3)-backbone being linked to a side D-glucose unit by a (1,6)-beta bond produced from, e.g., fungi of the Sclerotium spp.), SSG (a highly branched beta-(1,3)-glucan produced from, e.g., the fungus Sclerotinia sclerotiorum), soluble glucans from yeast (a beta-(1,3)-D-glucan with beta-(1,6)-linked side groups produced from, e.g., Saccharomyces cerevisiae), laminarin (a beta-(1,3)-glucan with beta-(1,3)-glucan and beta-(1,6)-glucan side groups produced from, e.g., the brown algae Laminaria digitata), and cereal glucans such as barley beta-glucans (linear beta-(1,3)(1,4)-D-glucan produced from, e.g., Hordeum vulgare, Avena sativa, or Triticum vulgare).

The beta-glucan can be scleroglucan, a branched beta-glucan with one out of three glucose molecules of the beta-(1,3)-backbone being linked to a side D-glucose unit by a (1,6)-beta bond produced from, e.g., fungi of the Sclerotium. The beta-glucan can be schizophyllan, a branched beta-glucan having one glucose branch for every third glucose residue in the beta-(1,3)-backbone produced from, e.g., the fungus Schizophyllan commune. Fungal strains that secrete such glucans are known to those skilled in the art. Examples include Schizophylium commune Sclerotium rolfsii, Sclerotium glucanicum, Monilinla fructigena, Lentinula edodes, or Botrygs cinera. The beta-glucan can have desirable characteristics for treatment of subterranean formations as described in co-pending patent applications U.S. Provisional Application Ser. Nos. 62/313,973, 62/313,988, 62/345,109, and 62/348,278, and U.S. Patent Publication No. 2012/0205099.

An aqueous solution including the refined beta-glucan can have desirable characteristics for treatment of subterranean formations as described in co-pending patent applications U.S. Provisional Application Ser. Nos. 62/313,973, 62/313,988, 62/345,109, and 62/348,278, and U.S. Patent Publication No. 2012/0205099.

The beta-glucan described herein can have any suitable molecular weight, such as about 300,000 to about 8 million Daltons, about 2 million to about 8 million Daltons, or about 4 million to about 6 million Daltons.

### Particle Size.

The refined beta-glucan can have any suitable particle size, such as a dry particle size (e.g., as a powder that is not dispersed in a liquid) of about 100 microns or less to about 1,000 microns or less, or to a particle size that is less than or equal to about 100 microns, 250 microns, or less than or equal to about 1,000 microns or more.

A dispersed mixture of the beta-glucan in water at a concentration of 1 mg/mL (e.g., formed by mixing the beta-glucan in the form of a powder with water in a concentration of 10 mg/mL at 20,000 rpm for 8 minutes and diluting the mixture with additional water to form a 1 mg/mL solution) can have an obscuration (i.e., 1/transmittance, such as at 633 nm and 470 nm) of less than 0.7%, or about 0.001% to about 0.7%, about 0.001% to about 0.6%, about 0.2% to about 0.6%, or about 0.001% or less, or less than, equal to, or greater than about 0.005, 0.01, 0.02, 0.03, 0.04, 0.05, 0.06, 0.08, 0.1, 0.15, 0.2, 0.25, 0.3, 0.35, 0.4, 0.45, 0.5, 0.55, 0.6, 0.65%, or about 0.7% or more.

The refined beta-glucan can be scleroglucan, and a dispersed mixture of the beta-glucan in water at a concentration of 1 mg/mL can have an obscuration of about 0.001% to about 0.5%, 0.01% to about 0.35%, about 0.25% to about 0.35%, or about 0.001% or less, or less than, equal to, or greater than about 0.005, 0.01, 0.02, 0.03, 0.04, 0.05, 0.06, 0.08, 0.1, 0.12, 0.14, 0.16, 0.18, 0.2, 0.22, 0.24, 0.25, 0.26, 0.27, 0.28, 0.29, 0.3, 0.31, 0.32, 0.33, 0.34, 0.35, 0.36, 0.38, 0.4, 0.42, 0.44, 0.46, 0.48, or about 0.5 wt % or more.

The refined beta-glucan can be schizophyllan, and a dispersed mixture of the beta-glucan in water at a concentration of 1 mg/mL can have an obscuration of less than or equal to about 0.7%, or about 0.001% to about 0.7%, about 0.3% to about 0.7%, about 0.4% to about 0.5%, or about 0.001% or less, or less than, equal to, or greater than about 0.005, 0.01, 0.02, 0.03, 0.04, 0.05, 0.06, 0.08, 0.1, 0.12, 0.14, 0.16, 0.18, 0.2, 0.22, 0.24, 0.25, 0.26, 0.27, 0.28, 0.29, 0.3, 0.32, 0.34, 0.36, 0.38, 0.4, 0.41, 0.42, 0.43, 0.44, 0.45, 0.46, 0.47, 0.48, 0.49, 0.5, 0.52, 0.54, 0.56, 0.58, 0.6, 0.62, 0.64, 0.68, or about 0.7% or more.

The beta-glucan as a dry powder not dispersed in a liquid can have any suitable particle size (e.g., number average particle size), such as about 0.01 microns to about 5,000 microns, or about 0.01 microns or less, or less than, equal to, or greater than about 0.1 microns, 1, 2, 3, 4, 5, 6, 8, 10, 12, 14, 16, 18, 20, 25, 30, 35, 40, 45, 50, 60, 70, 80, 90, 100, 125, 150, 175, 200, 225, 250, 275, 300, 350, 400, 450, 500, 600, 700, 800, 900, 1,000, 1,500, 2,000, 2,500, 3,000, 3,500, 4,000, 4,500, or about 5,000 microns or more.

The refined beta-glucan can be scleroglucan and can have a majority of (e.g., more than 50 wt % of) particles with a particle size of about 1.5 microns to about 500 microns and of about 700 microns to about 5,000 microns. The beta-glucan can be substantially free of particles having a particle size of greater than about 500 microns to less than about 700 microns, particles having a particle size greater than about 5,000 microns, and particles having a particle size of 0.01 microns to less than about 1.5 microns.

The refined beta-glucan can be schizophyllan and can have a majority of particles with a particle size of about 0.01 micron to about 0.8 microns and of about 1.05 micron to about 2,000 microns. The beta-glucan can be substantially free of particles having a particle size of greater than about 0.8 microns to less than about 1.05 microns and particles having a particle size greater than about 2,000 microns.

### Dynamic Mechanical Analysis.

The refined beta-glucan can have a T_{g} (glass transition temperature) as measured by onset of storage modulus change as detected by dynamic mechanical analysis that is about 50° C. to about 90° C., or about 60° C. to about 80° C., or about 50° C. or less, or less than, equal to, or greater than about 52° C., 54, 56, 58, 60, 62, 64, 66, 68, 70, 72, 74, 76, 78, 80, 82, 84, 86, 88° C., or about 90° C. or more.

The refined beta-glucan can be scleroglucan and can have a T_{g} as measured by onset of storage modulus change as detected by dynamic mechanical analysis of about 70° C. to about 80° C., about 72° C. to about 76° C., or about 70° C. or less, or less than, equal to, or greater than about 71° C., 72, 72.5, 73, 73.5, 74. 74.5, 75, 75.5, 76, 77, 78, 79° C., or about 80° C. or more.

The refined beta-glucan can be schizophyllan and can have a T_{g} as measured by onset of storage modulus change as detected by dynamic mechanical analysis that is about 60° C. to about 70° C., about 65° C. to about 66° C., or about 60° C. or less, or less than, equal to, or greater than about 60° C., 61, 62, 63, 64, 64.5, 65, 65.5, 66, 66.5, 67, 68, 69° C., or about 70° C. or more.

The refined beta-glucan can have a T_{g} as measured by the peak tan delta as detected by dynamic mechanical analysis of about 70° C. to about 110° C., about 85° C. to about 100° C., or about 70° C. or less, or less than, equal to, or greater than about 72° C., 74, 76, 78, 80, 82, 84, 86, 88, 90, 92, 94, 96, 98, 100, 102, 104, 106, 108° C., or about 110° C. or more.

The refined beta-glucan can be scleroglucan and can have a T_{g} as measured by the peak tan delta as detected by dynamic mechanical analysis of about 90° C. to about 105° C., about 97° C. to about 99° C., or about 90° C. or less, or less than, equal to, or greater than about 91° C., 92, 93, 94, 95, 96, 96.5, 97, 97.5, 98, 98.5, 99, 99.5, 100, 101, 102, 103, 104° C., or about 105° C. or more.

The refined beta-glucan can be schizophyllan and can have T_{g} as measured by the peak tan delta as detected by dynamic mechanical analysis of about 85° C. to about 95° C., about 89° C. to about 90° C., or about 85° C. or less, or less than, equal to, or greater than about 86° C., 87, 88, 88.5, 89, 89.5, 90, 90.5, 91, 92, 93, 94° C., or about 95° C. or more.

### Atomic Force Microscopy.

Atomic force microscopy images of the beta-glucan can be substantially free of monolithic globular domains (e.g., domains that are not fibrous) larger than about 4 microns, 3.5, 3, 2.5, 2, 1.5, 1, or larger than about 0.5 microns.

The refined beta-glucan can be scleroglucan and AFM images thereof can be substantially free of monolithic globular domains larger than about 1 micron.

The refined beta-glucan can be schizophyllan and AFM images thereof can be substantially free of monolithic globular domains larger than about 2 microns.

### Thermogravimetric Analysis.

The refined beta-glucan can have any suitable majority decomposition temperature (e.g., the temperature wherein the majority of the refined beta-glucan decomposes, which can be determined from an inflection point in a weight percent versus temperature plot of themogravimetric analysis data), such as about 300° C. to about 350° C., about 315° C. to about 340° C., or about 300° C. or less, or less than, equal to, or greater than about 305° C., 310, 315, 320, 325, 330, 335, 340, 345° C., or about 350° C. or more.

The refined beta-glucan can be scleroglucan and can have a majority decomposition temperature of about 330° C. to about 350° C., or about 335° C. to about 345° C., or about 330° C. or less, or less than, equal to, or greater than about 331° C., 332, 333, 334, 335, 336, 337, 338, 339, 340, 341, 342, 343, 344, 345, 346, 347, 348, 349° C., or about 350° C. or more.

The refined beta-glucan can be schizophyllan and can have a majority decomposition temperature of about 340° C. to about 355° C., about 345° C. to about 350° C., or about 340° C. or less, or less than, equal to, or greater than about 341° C., 342, 343, 344, 345, 346, 347, 348, 349, 350, 351, 352, 353, 354° C., or about 355° C. or more.

### Dry Matter.

Any suitable proportion of the beta-glucan can be dry matter (e.g., substantially free of liquids such as water, or organic solvent, or a combination thereof), such as about 80 wt % to about 98 wt % of the beta-glucan, about 88 wt % to about 94.5 wt % of the beta-glucan, or about 80 wt % or less, or less than, equal to, or greater than about 81 wt %, 82, 83, 84, 85, 86, 87, 88, 88.5, 89, 89.5, 90, 90.5, 91, 91.5, 92, 92.5, 93, 93.5, 94, 94.5, 95, 96, 97 wt %, or about 98 wt % or more.

### Viscosity Build.

A solution of the beta-glucan in water prepared by subjecting to a shear of about 260,000 s⁻¹ or 200,000 s⁻¹ for about 0.01 s to about 2 s can have a viscosity that is at least about 70% (e.g., at least 75%, 80, 85, 90, or 95% or more) of an ultimate viscosity of the solution. The ultimate viscosity of the solution is the highest possible viscosity of the solution having the same composition, and can be estimated as the viscosity of a solution of the beta-glucan in water prepared by subjecting to a shear of about 260,000 s⁻¹ for about 0.06 s to about 6 s, or 200,000 s⁻¹ for about 0.12 s to about 12 s.

### Filterability.

The Filterability Ratio of an aqueous composition including the refined beta-glucan can be about 1.01 to about 1.5, or about 1.08 to about 1.25, or about 1, or about 1.01 or less, or less than, equal to, or greater than about 1.02, 1.04, 1.06, 1.08, 1.10, 1.12, 1.14, 1.16, 1.18, 1.20, 1.22, 1.24, 1.26, 1.28, 1.3, 1.32, 1.34, 1.36, 1.38, 1.4, 1.42, 1.44, 1.46, 1.48, or about 1.5 or more. The Filterability Ratio can be determined by the procedure described in the Examples. The Filterability Ratio indicates the degree to which the mixture causes pore clogging over time, and is a ratio of time required for 20 g flow at a steady pressure through a filter at a later time divided by the time required for 20 g flow through the filter at an earlier time, with a ratio of 1 indicating no pore clogging (e.g., equal times required for flow at later and earlier times through the same filter at the same pressure). The Filterability Ratio can be determined by passing the sample through a filter having a pore size of about 1.2 microns (e.g., 47 mm diameter, 1.2 μm pore size, EMD Millipore mixed cellulose esters filter (part #RAWP04700)) using a pressure to achieve a flux of about 1-3 g/s and maintaining such pressure consistently while measuring the mass of filtrate produced. The Filterability Ratio is (time(180 g)−time (160 g))/(time(80 g)−time (60 g)). Prior to passing the sample through the 1.2 micron filter, the sample can first be optionally passed through a filter having a pore size of about 2 microns (e.g., 47 mm diameter Millipore AP25 filter (AP2504700)) at about 100-300 mL/min. The sample can optionally be prepared by combining powdered refined beta-glucan with water in a concentration of 1 g/L, mixing at 700 rpm for 20 minutes, and then agitating at 2,000 rpm for 4 hours.

A 2 g/L solution of the refined beta-glucan in water prepared by mixing at 260,000 s⁻¹ for about 0.06 s to about 6 s, or at 200,000 s⁻¹ for about 0.12 s to about 12 s, can have a Filterability Ratio that is about 1.01 to about 1.3, about 1.05 to about 1.3, or about 1.1 to about 1.25, or about 1.01 or less, or less than, equal to, or greater than about 1.02, 1.03, 1.04, 1.05, 1.06, 1.08, 1.10, 1.12, 1.14, 1.16, 1.18, 1.20, 1.22, 1.24, 1.26, 1.28 or about 1.3.

The refined beta-glucan can be scleroglucan, and a 2 g/L solution of the beta-glucan in water prepared by mixing at 260,000 s⁻¹ for about 0.06 s to about 6 s, or at 200,000 s⁻¹ for about 0.12 s to about 12 s, can have a Filterability Ratio that is about 1.01 to 1.2, about 1.1 to 1.2, or about 1.01 or less, or less than, equal to, or greater than about 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, or about 1.2 or more.

The refined beta-glucan can be schizophyllan, and a 2 g/L solution of the beta-glucan in water prepared by mixing at 260,000 s⁻¹ for about 0.06 s to about 6 s, or at 200,000 for about 0.12 s to about 12 s, can have a Filterability Ratio that is about 1.01 to 1.25, about 1.15 to 1.25, or about 1.01 or less, or less than, equal to, or greater than about 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, or about 1.25 or more.

### Viscosity Retention.

A 2 g/L solution of the refined beta-glucan in water prepared by mixing at 260,000 s⁻¹ for about 0.06 s to about 6 s, or at 200,000 s for about 0.12 s to about 12 s, has an original viscosity, and then subjecting the solution to filtration through a 1.2. micron filter provides a filtered solution that can have a viscosity that is about 90% to about 100% of the original viscosity, or about 95% to about 100% of the original viscosity, or about 90% or less, or less than, equal to, or greater than about 91%, 92, 93, 94, 95, 96, 97, 98, 99, 99.9%, or about 99.99% or more of the original viscosity.

The refined beta-glucan can be scleroglucan. A 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and then subjecting the solution to filtration through a 1.2 micron filter provides a filtered solution that can have a viscosity that is about 95% to about 100% of the original viscosity, about 98% to about 100%, about 99.5% to about 100%, or about 95% or less, or less than, equal to, or greater than about 95.5%, 96, 96.5, 97, 97.5, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9%, or about 99.99% or more of the original viscosity.

The refined beta-glucan can be schizophyllan. A 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and then subjecting the solution to filtration through a 1.2 micron filter provides a filtered solution that can have a viscosity that is about 94% to about 100% of the original viscosity, about 94% to about 99%, about 96% to about 98% of the original viscosity, or about 94% or less, or less than, equal to, or greater than about 94.2%, 94.4, 94.6, 94.8, 95, 95.2, 95.4, 95.6, 95.8, 96, 96.2, 96.4, 96.6, 96.8, 97, 97.2, 97.4, 97.6, 97.8, 98, 98.2, 98.4, 98.6, 98.8%, or about 99% or more of the original viscosity.

### Inductively Coupled Plasma Atomic Emission Spectroscopy.

The refined beta-glucan can have a total atomic calcium content of about 300 μg/g to about 10,000 μg/g, about 500 μg/g to about 9,000μg/g, or about 300 μg/g or less, or less than, equal to, or greater than about 400 μg/g, 500, 600, 700, 800, 900, 1,000, 2,000, 3,000, 4,000, 5,000, 6,000, 7,000, 8,000, 9,000 μg/g, or about 10,000 μg/g or more.

The refined beta-glucan can be scleroglucan and can have a total atomic calcium content of about 300 μg/g to about 4,500 μg/g, about 500 μg/g to about 4,100 μg/g, or about 300 μg/g or less, or less than, equal to, or greater than about 400 μg/g, 500, 600, 700, 800, 900, 1,000, 2,000, 3,000, 3,500, 3,700, 3,800, 3,900, 4,000, 4,100, 4,200, 4,300, 4,400 μg/g, or about 4,500 μg/g or more.

The refined beta-glucan can be schizophyllan and can have a total atomic calcium content of about 7,000 μg/g to about 10,000 μg/g, about 8,000 μg/g to about 9,000 μg/g, or about 7,000 μg/g or less, or less than, equal to, or greater than about 7,200 μg/g, 7,400, 7,600, 7,800, 8,000, 8,100, 8,200, 8,300, 8,400, 8,500, 8,600, 8,700, 8,800, 8,900, 9,000, 9,200, 9,400, 9,600, 9,800 μg/g, or about 10,000 μg/g or more.

The refined beta-glucan can have a total atomic copper content of about 0 μg/g to about 4 μg/g about 0 μg/g to about 3 μg/g, or about 0.1 μg/g or less, or less than, equal to, or greater than about 0.2 μg/g, 0.3, 0.4, 0.5, 0.6, 0.7, 0.8, 1, 1.2, 1.4, 1.6, 1.8, 2, 2.2, 2.4, 2.6, 2.8, 3, 3.2, 3.4, 3.6, 3.8 μg/g, or about 4 μg/g or more.

The refined beta-glucan can be scleroglucan and can have a total atomic copper content of about 0 μg/g to about 4 μg/g, about 0 μg/g to about 3.5 μg/g, or about 0.1 μg/g or less, or less than, equal to, or greater than about 0.2 μg/g, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9 μg/g, or about 4 μg/g or more.

The refined beta-glucan can be schizophyllan and can have a total atomic copper content of about 0.5 μg/g to about 2 μg/g, about 1.1 μg/g to about 1.5 μg/g, or about 0.5 μg/g or less, or less than, equal to, or greater than about 0.6 μg/g, 0.7, 0.8, 0.9, 1, 1.1, 1.2, 1.3, 1.4, 1.5, 16, 1.7, 18, 1.9 μg/g, or about 2 μg/g or more.

The refined beta-glucan can have a total atomic iron content of about 10 μg/g to about 300 μg/g, about 40 μg/g to about 290 μg/g, or about 10 μg/g or less, or less than, equal to, or greater than about 20 μg/g, 40, 60, 80, 100, 120, 140, 160, 180, 200, 220, 240, 260, 280 μg/g, or about 300 μg/g or more.

The refined beta-glucan can be scleroglucan and can have a total atomic iron content of about 150 μg/g to about 300 μg/g, about 160 μg/g to about 290 μg/g, or about 150 μg/g or less, or less than, equal to, or greater than about 155 μg/g, 160, 165, 170, 180, 190, 200, 210, 220, 230, 240, 250, 260, 265, 270, 275, 280, 285, 290, 295 or about 300 μg/g or more.

The refined beta-glucan can be schizophyllan and can have a total atomic iron content of about 30 μg/g, to about 80 μg/g, about 45 μg/g to about 60 μg/g, or about 30 μg/g or less, or less than, equal to, or greater than about 30 μg/g, 35, 40, 45, 50, 55, 60, 65, 70, 75 μg/g, or about 80 μg/g, or more.

The refined beta-glucan can have a total atomic potassium content of about 0 μg/g to about 500 μg/g, about 0 μg/g to about 300 μg/g, or about 0 μg/g, or about 50 μg/g or less, or less than, equal to, or greater than about 60 μg/g, 70, 80, 90, 100, 110, 120, 130, 140, 150, 200, 250, 300, 350, 400, 450 μg/g, or about 500 μg/g or more.

The refined beta-glucan can be scleroglucan and can have a total atomic potassium content of about 0 μg/g to about 200 μg/g, about 0 μg/g to about 125 μg/g, or about 0 μg/g, or about 10 μg/g or less, or less than, equal to, or greater than about 20 μg/g, 30, 40, 50, 60, 70, 80, 100, 110, 120, 130, 140, 150, 160, 170, 180, 190 μg/g, or about 200 μg/g or more.

The refined beta-glucan can be schizophyllan and can have a total atomic potassium content of about 25 μg/g to about 310 μg/g, about 260 μg/g to about 300 μg/g, or about 250 μg/g or less, or less than, equal to, or greater than about 260 μg/g, 265, 270, 275, 280, 285, 290, 295, 300, 305 μg/g, or about 310 μg/g or more.

The refined beta-glucan can have a total atomic magnesium content of about 1 μg/g to about 14,000 μg/g, about 5 μg/g to about 13,000 μg/g, or about 1 μg/g or less, or less than, equal to, or greater than about 2 μg/g, 4, 6, 8, 10, 20, 30, 40, 50, 60, 80, 100, 150, 200, 250, 300, 400, 500, 600, 800, 1,000, 1,200, 1,400, 1,600, 1,800, 2,000, 2,500, 3,000, 4,000, 5,000, 6,000, 8,000, 10,000, 12,000 μg/g, or about 14,000 μg/g or more.

The refined beta-glucan can be scleroglucan and can have a total atomic magnesium content of about 1 μg/g to about 100 μg/g, about 5 μg/g to about 50 μg/g, or about 1 μg/g or less, or less than, equal to, or greater than about 2 μg/g, 4, 6, 8, 10, 12, 14, 16, 18, 20, 22, 24, 26, 28, 30, 32, 34, 36, 38, 40, 45, 50, 55, 60, 65, 70, 75, 80, 85, 90, 95 μg/g, or about 100 μg/g or more.

The refined beta-glucan can be schizophyllan and can have a total atomic magnesium content of about 12,000 μg/g to about 14,000 μg/g, about 12,800 μg/g to about 12,900 μg/g, or about 12,000 μg/g or less, or less than, equal to, or greater than about 12,100 μg/g, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, or about 14,000 μg/g, or more.

The refined beta-glucan can have a total atomic manganese content of about 0.1 μg/g to about 30 μg/g, about 0.2 μg/g to about 20 μg/g, or about 0.1 μg/g or less, or less than, equal to, or greater than about 0.2 μg/g, 0.3, 0.4, 0.5, 0.6, 0.8, 1,1.2,1.4,1.6,1.8,2,4,6,8,10,12,14,16,18,20,22,24,26, 28 μg/g, or about 30 μg/g or more.

The refined beta-glucan can be scleroglucan and can have a total atomic manganese content of about 0.1 μg/g to about 2 μg/g, about 0.2 μg/g to about 1.9 μg/g, or about 0.1 μg/g or less, or less than, equal to, or greater than about 0.2 μg/g, 0.3, 0.4, 0.5, 0.6, 0.7, 0.8, 0.9, 1, 1.1, 1.2, 1.3, 1.4, 1.5, 1.6, 1.7, 1.8, 1.9 μg/g, or about 2 μg/g or more.

The refined beta-glucan can be schizophyllan and can have a total atomic manganese content of about 14 μg/g to about 25 μg/g, about 16 μg/g to about 22 μg/g, or about 14 μg/g or less, or less than, equal to, or greater than about 15 μg/g, 16, 17, 18, 19, 20, 21, 22, 23, 24 μg/g, or about 25 μg/g or more.

The refined beta-glucan can have a total atomic sodium content of about 100 μg/g, to about 4,000 μg/g, about 200 μg/g to about 3,200 μg/g, or about 100 μg/g or less, or less than, equal to, or greater than about 200 μg/g, 300, 400, 500, 600, 800, 1,000, 1,500, 2,000, 2,500, 3,000, 3,500 μg/g, or about 4,000 μg/g or more.

The refined beta-glucan can be scleroglucan and can have a total atomic sodium content of about 100 μg/g to about 3,500 μg/g, about 250 μg/g to about 3,200 μg/g, or about 100 μg/g or less, or less than, equal to, or greater than about 200 μg/g, 250, 300, 400, 500, 600, 800, 1,000, 1,500, 2,500, 2,700, 2,800, 2,900, 3,000, 3,100, 3,200, 3,300, 3,400 μg/g, or about 3,500 μg/g or more.

The refined beta-glucan can be schizophyllan and can have a total atomic sodium content of about 150 μg/g to about 350 μg/g, about 200 μg/g to about 300 μg/g, or about 150 μg/g or less, or less than, equal to, or greater than about 160 μg/g, 170, 180, 190, 200, 210, 220, 230, 240, 250, 260, 270, 280, 290, 300, 310, 320, 330, 340 μg/g, or about 350 μg/g or more.

The refined beta-glucan can have a total atomic phosphorus content of about 0 μg/g to about 15,000 μg/g, about 0 μg/g to about 12,000 μg/g, or about 0 μg/g, or about 100 μg/g or less, or less than, equal to, or greater than about 200 μg/g, 300, 400, 500, 600, 800, 1,000, 1,500, 2,000, 3,000, 4,000, 5,000, 6,000, 7,000, 8,000, 9,000, 10,000, 11,000, 12,000, 13,000, 14,000 μg/g, or about 15,000 μg/g or more.

The refined beta-glucan can be scleroglucan and can have a total atomic phosphorus content of about 0 μg/g, to about 500 μg/g, about 0 μg/g to about 300 μg/g, or about 0 μg/g, or about 10 or less, or less than, equal to, or greater than about 20 μg/g, 30, 40, 50, 60, 70, 80, 90, 100, 110, 120, 130, 140, 150, 160, 170, 180, 190, 200, 220, 240, 260, 280, 290, 300, 350, 400, 450 μg/g, or about 500 μg/g or more.

The refined beta-glucan can be schizophyllan and can have a total atomic phosphorus content of about 10,00 μ/g to about 12,000 μg/g, about 10,500 μg/g to about 11,500 μg/g, or about 10,000 μg/g or less, or less than, equal to, or greater than about 10,100 μg/g, 10,200, 10,300, 10,400, 10,500, 10,600, 10,700, 10,800,10,900, 11,000, 11,100, 11,200, 11,300, 11,400, 11,500, 11,600, 11,700, 11,800, 11,900 μg/g or about 12,000 μg/g or more.

The refined beta-glucan can have a total atomic sulfur content of about 50 μg/g to about 400 μg/g, about 100 ps/g, to about 350 μg/g, or about 50 μg/g or less, or less than, equal to, or greater than about 60 μg/g, 80, 100, 120, 140, 160, 180, 200, 220, 240, 260, 280, 300, 320, 340, 360, 380 or about 400 μg/g or more.

The refined beta-glucan can be scleroglucan and can have a total atomic sulfur content of about 50 μg/g to about 300 μg/g, about 100 μg/g to about 250 μg/g, or about 50 μg/g or less, or less than, equal to, or greater than about 60 μg/g, 70, 80, 90, 100, 110, 120, 130, 140, 150, 160, 170, 180, 190, 200, 210, 220, 230, 240, 250, 260, 270, 280, 290 μg/g, or about 300 μg/g or more.

The refined beta-glucan can be schizophyllan and can have a total atomic sulfur content of about 200 μg/g to about 400 μg/g, about 250 μg/g to about 350 μg/g, or about 200 μg/g or less, or less than, equal to, or greater than about 210 μg/g, 220, 230, 240, 250, 260, 270, 280, 290, 300, 310, 320, 330, 340, 350, 360, 370, 380, 390 μg/g, or about 400 μg/g or more.

The refined beta-glucan can have a total atomic zinc content of about 0 μg/g to about 15 μg/g, about 0 μg/g to about 13 μg/g, or about 0 μg/g, or less than, equal to, or greater than about 0.5 μg/g, 1, 1.5, 2, 2.5, 3, 4, 5, 6, 7, 8, 9, 10, 11, 12, 13, 14 μg/g, or about 15 μg/g or more.

The refined beta-glucan can be scleroglucan and can have a total atomic zinc content of about 0 μg/g to about 4 μg/g, about 0 μg/g to about 3 μg/g, or about 0 μg/g, or less than, equal to, or greater than about 0.5 μg/g, 1, 1.5, 2, 2.5, 3, 3.5 μg/g, or about 4 μg/g, or more.

The refined beta-glucan can be schizophyllan and can have a total atomic zinc content of about 10 μg/g to about 16 μg/g, about 12 μg/g, to about 14 μg/g, or about 10 μg/g or less, or less than, equal to, or greater than about 10.5 μg/g, 11, 11,5, 12,5, 13, 13.5, 14, 14.5, 15, 15.5 μg/g, or about 16 μg/g or more.

### Protein Content.

The refined beta-glucan can have any suitable protein content, such as about 0.01 wt % to about 2 wt % of the beta-glucan, about 0.10 wt % to about 0.45 wt %, or about 0.01 wt % or less, or less than, equal to, or greater than 0.05, 0.1, 0.15, 0.2, 0.25, 0.3, 0.35, 0.4, 0.45, 0.5, 0.6, 0.7, 0.8, 0,9, 1, 1.1, 1.2, 1.3, 1.4, 1.5, 1.6, 1.7, 1.8, 1.9 wt %, or about 2 wt % or more.

The refined beta-glucan can be scleroglucan and the protein content can be about 0.05 wt % to about 0.3 wt % of the beta-glucan, about 0.10 wt % to about 0.20 wt %, about 0.05 wt % or less, or less than, equal to, or greater than about 0.06 wt %, 0.07, 0.08, 0.09, 0.1, 0.11, 0.12, 0.13, 0.14, 0.15, 0.16, 0.17, 0.18, 0.19, 0.2, 0.22, 0.24, 0.26, 0.28, or about 0.3 wt % or more of the beta-glucan.

The refined beta-glucan can be schizophyllan and the protein content can be about 0.2 wt % to about 0.6 wt % of the beta-glucan, about 0.35 wt % to about 0.45 wt %, or about 0.2 wt % or less, or less than, equal to, or greater than about 0.25 wt %, 0,3, 0,31, 032, 033, 034, 0.35, 0.36, 0.37, 0.38, 0.39, 0.4, 0.41, 0.42, 0.43, 0.44 0.45, 0.46 0.47, 0.48, 0.49, 0.5, 0.55 wt %, or about 0.6 wt % or more.

### Total Nitrogen.

The refined beta-glucan can have any suitable total atomic nitrogen content, such as about 1 μg/g to about 10 μg/g, about 2μg/g to about 7 μg/g, or about 1 μg/g or less, or less than, equal to, or greater than about 2 μg/g, 3, 4, 5, 6, 7, 8, 9 μg/g, or about 10 μg/g or more.

The refined beta-glucan can be scleroglucan and can have a total atomic nitrogen content of about 1 μg/g to about 5 μg/g, about 2.5 μg/g to about 3 μg/g, or about 1 μg/g or less, or less than, equal to, or greater than about 1.5 μg/g, 2, 2.5, 3, 3.5, 4, 4.5, 5 μg/g or about 5 μg/g or more.

The refined beta-glucan can be schizophyllan and can have a total atomic nitrogen content of about 4 μg/g to about 8 μg/g, about 5.5 μg/g to about 6.5 μg/g, or about 4 μg/g or less, or less than, equal to, or greater than about 4 μg/g, 4.5, 5, 5.5, 6, 6.5, 7, 7.5 μg/g, or about 8 μg/g or more.

### Ash Content.

The refined beta-glucan can have any suitable ash content, as a weight percent of the pre-combusted refined beta-glucan. For example, upon total combustion, the beta-glucan can form an ash that is less than about 3 wt % of the (pre-combusted) beta-glucan, or less than about 0.5 wt %, or about 0.01 wt % to about 3 wt %, about 0.1 wt % to about 1.3 wt %, about 0.1 wt % to about 1.2 wt %, about 0.001 wt % or less, or less than, equal to, or greater than about 0.01, 0.1, 0.2, 0.3, 0.4, 0.5, 0.6, 0.7, 0.8, 0.9, 1, 1.1, 1.2, 1.3, 1.4, 1.5, 1.6, 1,7, 1.8, 1.9, 2, 2.1, 2.2, 2.3, 2.4, 2.5, 2.6, 2.7, 2.8, 2.9, or about 3 wt % or more of the beta-glucan.

### Sand Pack.

The refined beta-glucan can provide reduced increase in pressure drop across a sand-packed column over time compared to other viscosifiers. For example, a dispersed mixture of the beta-glucan in water at a concentration of 1 mg/mL, experiences less than or equal to a 50% increase in pressure drop across a sand-packed column having a total pore volume equal to one Sand Column Void Space Volume during passage of 200 Sand Column Void Space Volumes of the dispersed mixture through the sand-packed column, or an about 0.1% to about 50% increase in pressure drop, an about 1% to about 10% increase in pressure drop, or about 0.1% or less, or less than, equal to, or greater than about 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 7, 8, 9, 10, 12, 14, 15, 16, 20, 25, 30, 35, 40%, or about 50% or more.

During measurement of the pressure drop across the sand-packed column, the dispersed mixture of the beta-glucan in water can be at a relatively stable flow rate during the counting of the Sand Column Void Space Volumes. For example, water can be passed through the column first until a stable flow rate is achieved, and then the dispersed mixture of the beta-glucan in water can be passed through the column. At the beginning of the counting of the Sand Column Void Space Volumes, the sand-packed column can be substantially fresh and free of viscosifiers, such that no viscosifiers have yet caused any significant plugging of the sand-packed column to cause a decrease in the pressure drop across the column.

The sand-packed column used to measure the change in pressure drop during passage of the dispersed mixture of the beta-glucan therethrough can have any suitable permeability. For example, the sand-packed column can have a permeability of about 0.001 Darcy to about 30 Darcy, about 1.5 Darcy to about 5 Darcy, about 1 Darcy to about 4 Darcy, about 2 Darcy to about 2.5 Darcy, or about 0.001 Darcy or less, or less than, equal to, or greater than about 0.002 Darcy, 0.004, 0.006, 0.008, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.6, 0.7, 0.8, 0.9, 1, 1.2, 1.4, 1.6, 1.8, 2, 2.1, 2.2, 2.3, 2.4, 2.5, 2.6, 2.7, 2.8, 2.9, 3, 3.2, 3.4, 3.6, 3.8, 4, 4.5, 5, 5.5, 6, 7, 8, 9, 10, 12, 14, 16, 18, 20, 25, or about 30 Darcy or more.

The dispersed mixture of the beta-glucan in water used to characterize the reduced increase in pressure drop across the sand-packed column can be a dispersed mixture of the beta-glucan in salt water. The salt water can be any suitable salt water, such as brine, produced water, flowback water, brackish water, sea water, synthetic sea water, or a combination thereof. The one or more salts therein can be any suitable salt, such as at least one of NaBr, CaCl₂, CaBr₂, ZnBr₂, KCl, NaCl, a carbonate salt, a sulfonate salt, sulfite salts, sulfide salts, a phosphate salt, a phosphonate salt, a magnesium salt, a sodium salt, a calcium salt, a bromide salt, a formate salt, an acetate salt, a nitrate salt, or a combination thereof. The salt water can have a total dissolved solids level of about 1,000 mg/L to about 250,000 mg/L, about 20,000 mg/L to about 50,000 mg/L, or about 1,000 mg/L, or less, or about 0 mg/L, or about 5,000 mg/L, 10,000, 15,000, 20,000, 25,000, 30,000, 35,000, 40,000, 50,000, 75,000, 100,000, 125,000, 150,000, 175,000, 200,000, 225,000, or about 250,000 mg/L or more, such as the total level of dissolved solids from a sea salt.

The dispersed mixture of the beta-glucan in water used to characterize the reduced increase in pressure drop across the sand-packed column can be passed through the sand-packed column at any suitable flow rate. For example, in an embodiment wherein the column is about 2.5 cm in diameter and about 15 cm in length, the flow rate can be 0.01 mL/min to about 100 mL/min, 0.1 to 10 mL/min, 0.5 mL/min to about 2 mL/min, or about 0.01 mL/min or less, or less than, equal to, or greater than about 0.1 mL/min, 0.5, 1, 1.5, 2, 2.5, 3, 4, 5, 6, 7, 8, 9, 10, 12, 14, 16, 18, 20, 25, 50, 75, or about 100 mL/min or more. The flow rate can be generalized to any sized column in terms of the number of Sand Column Void Space Volumes of the flowed dispersed mixture of the beta-glucan per time, such as about 0.01 to about 10 Sand Column Void Space Volumes/min, about 0.01 to about 1, about 0.1 to about 0.3, or about 0.01 or less, or less than, equal to, or greater than about 0.02 Sand Column Void Space Volumes/min, 0.04, 0.06, 0.08, 0.1, 0.15, 0.2, 0.25, 0.3, 0.35, 0.4, 0.5, 0.6, 0.7, 0.8, 0.9, 1, 1.2, 1.4, 1.6, 1.8, 2, 2.5, 3, 3.5, 4, 4.5, 5, 6, 7, 8, 9, or about 10 Sand Column Void Space Volumes/min or more.

### Oxalic Acid Concentration.

The refined beta-glucan can have any suitable oxalic acid concentration, such as about 0 ppm to about 2,000 ppm, about 5 ppm to about 1,000 ppm, about 10 ppm to about 500 ppm, about 20 ppm to about 100 ppm, about 30 ppm to about 70 ppm, about 40 ppm to about 500 ppm, about 50 ppm to about 400 ppm, about 52 ppm to about 377 ppm, about 75 ppm to about 100 ppm, or about 0 ppm, or less than, equal to, or greater than about 5 ppm, 10, 15, 20, 25, 30, 35, 40, 45, 50, 51, 52, 53, 54, 55, 60, 65, 70, 75, 80, 85, 90, 95, 100, 120, 140, 160, 180, 200, 250, 300, 350, 400, 500, 750, 1,000, 1,500, or about 2,000 ppm or more. The oxalic acid concentration can be less than, equal to, or greater than about 52 ppm, 75, 76, 77, 88, 97, 118, 121, 132, 137, 147, 171, 218, 321, or about 377 ppm.

### Bulk Density.

The refined beta-glucan can have any suitable bulk density. For example, the refined beta-glucan can have a bulk density of about 0.2 kg/L to about 0.6 kg/L, or about 0.3 kg/L to about 0.5 kg/L, or about 0.2 kg/L or less, or less than, equal to, or greater than 0.25 kg/L, 0.3, 0.32, 0.34, 0.36, 0.38, 0.39, 0.4, 0.41, 0.42, 0.44, 0.46, 0.48, 0.5, 0.55 kg/L, or about 0.6 kg/L or more. The bulk density can be determined by any suitable method, such as by weighing a volume of 200 mL of powder.

### Composition Including the Refined Beta-Glucan.

Various aspects of the present invention provide a composition that includes the refined beta-glucan. The refined beta-glucan can be any suitable aspect of the refined beta-glucan described herein. The composition can be a solid (e.g., a powder), a liquid (e.g., an aqueous liquid, an organic liquid, or a combination thereof), or a combination thereof (e.g., a suspension of the solid refined beta-glucan in a liquid, or a partially dissolved solution of the refined beta-glucan).

The composition can be a liquid, such as an aqueous liquid (e.g., having 50 wt % or more water therein), or an organic liquid (e.g., having 50 wt % or more organic liquid therein, such as an alcohol, an alpha-hydroxy acid alkyl ester, a polyalkylene glycol alkyl ether, or a combination thereof. The beta-glucan can be any suitable proportion of the liquid, such as about 0.001 wt % to about 99.999 wt % of the liquid, or about 0.001 wt % or less, or less than, equal to, or greater than about 0.01 wt %, 0.1, 1, 2, 3, 4, 5, 6, 8, 10, 12, 14, 16, 18, 20, 25, 30, 35, 40, 45, 50, 55, 60, 65, 70, 75, 80, 85, 90, 91, 92, 93, 94, 95, 96, 97, 98, 99, 99.9, 99.99, or about 99.999 wt % or more. The liquid can be a liquid for treating a subterranean formation (e.g., for enhanced oil recovery polymer flooding, for hydraulic fracturing, water shut-off, conformance, or a combination thereof), or a concentrated liquid designed to be diluted to form a liquid for treating a subterranean formation.

An aqueous composition can include water as any suitable proportion thereof, such as about 70 wt % to about 99.999 wt %, or about 95 wt % to about 99.99 wt %, or about 70 wt % or less, or less than, equal to, or greater than about 75 wt %, 80, 82, 84, 86, 88, 90, 91, 92, 93, 94, 95, 96, 97, 98, 99, 99.9, 99.99 wt %, or about 99.999 wt % or more. The water can include fresh water, salt water, brine, produced water, flowback water, brackish water, sea water, synthetic sea water, or a combination thereof. For a salt water, the one or more salts therein can be any suitable salt, such as at least one of NaBr, CaCl₂, CaBr₂, ZnBr₂, KCl, NaCl, a carbonate salt, a sulfonate salt, sulfite salts, sulfide salts, a phosphate salt, a phosphonate salt, a magnesium salt, a sodium salt, a calcium salt, a bromide salt, a formate salt, an acetate salt, a nitrate salt, or a combination thereof. The water can have any suitable total dissolved solids level, such as about 1,000 mg/L to about 250,000 mg/L, or about 1,000 mg/L or less, or about 0 mg/L, or about 5,000 mg/L, 10,000, 15,000, 20,000, 25,000, 30,000, 40,000, 50,000, 75,000, 100,000, 125,000, 150,000, 175,000, 200,000, 225,000, or about 250,000 mg/L or more. The water can have any suitable salt concentration, such as about 1,000 ppm to about 300,000 ppm, or about 1,000 ppm to about 150,000 ppm, or about 0 ppm, or about 1,000 ppm or less, or about 5,000 ppm, 10,000, 15,000, 20,000, 25,000, 30,000, 40,000, 50,000, 75,000, 100,000, 125,000, 150,000, 175,000, 200,000, 225,000, 250,000, 275,000, or about 300,000 ppm or more. In some examples, the water can have a concentration of at least one of NaBr, CaCl₂, CaBr₂, ZnBr₂, KCl, and NaCl of about 0.1% w/v to about 20% w/v, or about 0%, or about 0.1% w/v or less, or about 0.5% w/v, 1, 2, 3, 4, 5, 6, 7, 8, 9, 10, 11, 12, 13, 14, 15, 16, 17, 18, 19, 20, 21, 22, 23, 24, 26, 27, 28, 29, or about 30% w/v or more.

In some aspects, the composition is a solid, such as a powder. The refined beta-glucan can be any suitable proportion of the solid, such as about 0.001 wt % to about 99.999 wt % of the solid, or about 0.001 wt % or less, or less than, equal to, or greater than about 0.01 wt %, 0.1, 1, 2, 3, 4, 5, 6, 8, 10, 12, 14, 16, 18, 20, 25, 30, 35, 40, 45, 50, 55, 60, 65, 70, 75, 80, 85, 90, 91, 92, 93, 94, 95, 96, 97, 98, 99, 99,9, 99,99, or about 99.999 wt % or more.

### Method of Making Refined Beta-Glucan.

Various aspects of the present invention provide a method of making a refined beta-glucan. The method can include filtering a solution of a crude beta-glucan (e.g., an aqueous solution), to form a filtrate. The refined beta-glucan can be precipitated from the filtrate. The refined beta-glucan made by the method can be any suitable beta-glucan that can be made by the method, such as any refined beta-glucan described herein. Various aspects of the present invention provide a refined beta-glucan made by the method of making the refined beta-glucan described herein.

The solution of the crude beta-glucan can be provided prior to the onset of the method. The solution of the crude beta-glucan can be a substantially homogeneous solution. In some aspects, the method includes preparing the solution of the crude beta-glucan, such as including homogenizing a mixture of the crude beta-glucan and water. The crude beta-glucan can be any suitable crude beta-glucan, such as a fermentation product of microorganisms that formed the beta-glucan. The method can include homogenizing a mixture of the crude beta-glucan and water, to form the solution of the crude beta-glucan. The homogenizing can occur at any suitable temperature, such as ambient temperature, or such as at about 40° C. to about 90° C., or about 60° C. to about 85° C., or about 40° C. or less, or less than, equal to, or greater than about 45° C., 50, 55, 60, 65, 70, 75, 80, 85° C., or about 90° C. or more. The solution of the crude beta-glucan can have any suitable pH, such as about 4 to about 7, about 5 to about 6, or about 4 or less, or less than, equal to, or greater than about 4.5, 5, 5.5, 6, 6.5, or about 7 or more.

The method can include acidifying the solution of the crude beta-glucan prior to filtration. The acidifying can include adding a suitable acid (e.g., HCl) to decrease the pH of the solution to about 1 to about 4.5, about 1.5 to about 3.5, about 1.5 to about 2.5, or about 1 or less, or less than, equal to, or greater than about 1.5, 2, 2.5, 3, 3.5, 4, or about 4.5 or more. With the addition of various salts, such as calcium chloride, the acidifying can cause various materials to precipitate from the solution, such as oxalic acid (e.g., as a salt thereof such as calcium oxalate). After the acidifying, the solution can be agitated for a suitable duration. After the acidifying, the pH of the solution can be restored prior to filtration by addition of a suitable base (e.g., Na₂CO₃ or NaOH), such as to a pH of about 4 to about 7, about 5 to about 6, or about 4 or less, or less than, equal to, or greater than about 4.5, 5, 5.5, 6, 6.5, or about 7 or more. Restoring the pH of the solution can increase precipitation, such as the precipitation of the oxalic acid salt.

The filtering can be any suitable filtering that separates at least some materials from the crude beta-glucan. The filtering can include filtering the solution through a filter, such as any suitable filter, such as a filter having a permeability (e.g., water permeability) equal to the filter aid permeability values described herein. The filter can have any suitable pore size, such as a pore size of about 0.001 microns to about 1,000 microns, or about 0.1 microns to about 100 microns, or about 0.001 microns or less, or less than, equal to, or greater than about 0.01 microns, 0.1, 1, 2, 3, 4, 5, 6, 8, 10, 12, 14, 16, 18, 20, 25, 30, 35, 40, 45, 50, 60, 70, 80, 90, 100, 125, 150, 175, 200, 225, 250, 275, 300, 350, 400, 450, 500, 600, 700, 800, 900, or about 1,000 microns or more. The filtration can be conducted at any suitable temperature (e.g., having the solution at any suitable temperature, and optionally including temperature control of the filtration apparatus such as via jacketed filters), such as at ambient temperature, or such as at about 40° C. to about 90 ° C., or about 60° C. to about 85° C., or about 75° C. to about 85° C., or about 40° C. or less, or less than, equal to, or greater than about 45° C., 50, 55, 60, 65, 70, 75, 80, 85° C., or about 90° C. or more

The filtering can include adding one or more filter aids to the solution prior to filtering the solution through the filter. The filtering can include filtering all or a portion of the solution including the one or more filter aids through the filter to form a filter cake on the filter, and then returning the filtrate to the filter and filtering all of the solution through the filter cake on the filter. In some aspects, the filtering can includes filtering all or a portion of the solution including the one or more filter aids through the filter to form a filer cake on the filter, adding additional filter aid to the filtrate (e.g., finer filter aid than the filter aid in the filter cake that has been formed), filtering all or a portion of the solution with the additional aid through the filter cake to form a second filter cake (e.g., including a filter cake of fine filter aid on top of a filter cake of coarser filter aid), returning all the filtrate to the filter, and then filtering all of the solution through the second filter cake on the filter.

The one or more filter aids can be independently added at any suitable concentration to the solution, such as at about 1 g/L, to about 100 g/L, about 2 g/L to about 50 g/L, or about 1 g/L or less, or less than, equal to, or greater than about 2 g/L, 3, 4, 5, 6, 8, 10, 12, 14, 16, 18, 20, 25, 30, 35, 40, 45, 50, 55, 60, 65, 70, 75, 80, 85, 90, 95, or about 100 g/L or more. The one or more filter aids can independently be any suitable filter aid, such as a filter aid including diatomaceous earth, perlite, cellulose or cellulose derivatives, or a combination thereof. The one or more filter aids can independently have any suitable permeability (e.g., water permeability), such as about 0.001 Darcy to about 30 Darcy. The filter aid can be a coarse filter aid having a permeability of about 1 Darcy to about 30 Darcy, about 1.5 Darcy to about 5 Darcy, 1 Darcy to about 4 Darcy, or about 1 Darcy or less, or less than, equal to, or greater than about 1.2 Darcy, 1.4, 1.6, 1.8, 2, 2.2, 2.4, 2.6, 2.8, 3, 3.2, 3.4, 3.6, 3.8, 4, 4.5, 5, 5.5, 6, 7, 8, 9, 10, 12, 14, 16, 18, 20, 25, or about 30 Darcy or more. The filter aid can be a fine filter aid having a permeability of about 0.001 Darcy to about 1 Darcy, or about 0.02 Darcy to about 0.200 Darcy, or about 0.001 Darcy or less, or less than, equal to, or greater than about 0.002 Darcy, 0.004, 0.006, 0.008, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.6, 0.7, 0.8, 0.9, or about 1.0 Darcy or more.

The filtering can include performing multiple cycles of the filtration. The filter can be cleaned after each cycle of filtration (e.g., the filter cake can be removed, optionally squeezing liquid from therein). The filtering can include performing about 1 to about 10, or about 2 to about 5 cycles of filtration, or about 3 cycles of filtration. The filter aid used in each cycle of the filtration can be the same or different. In some aspects, the first filtration cycle can include no filter aid or only coarse filter aid or a combination of coarse and fine filter aids, while later filtration cycles include only fine filter aid or a combination of coarse and fine filter aids.

After the filtration is complete, the method can include precipitating biopolymer from the final filtrate. The precipitating can include adding a solvent to the filtrate that is miscible with water but in which the biopolymer has poor solubility, such as an organic solvent, for example, an alcohol (e.g, isopropyl alcohol). After adding the solvent, the mixture can be agitated for a suitable time to allow precipitation to occur. The precipitated biopolymer can be separated from the liquid. The precipitated biopolymer can optionally be washed, such as with an organic solvent (e.g., an alcohol, such as isopropyl alcohol), and the wash liquid can be drained away from the washed precipitated biopolymer.

After precipitation of the biopolymer, the method can further include drying the biopolymer, such as thermally, mechanically, or a combination thereof. Drying can include drying the biopolymer to a dry matter content of about 80 wt % to about 98 wt %, or about 85 wt % to about 95 wt %, or about 80 wt % or less, or about 81 wt %, 82, 83, 84, 85, 86, 87, 88, 89, 90, 91, 92, 93, 94, 95, 96, 97 wt %, or about 98 wt % or more.

The method can include grinding the product, such as to a particle size of about 100 microns or less to about 1,000 microns or less, or to a particle size that is less than or equal to about 100 microns, 250 microns, or less than or equal to about 1,000 microns or more.

### Method of Treating a Subterranean Formation.

Various aspects of the present invention provide a method of treating a subterranean formation. The method can include placing the refined beta-glucan described herein in the subterranean formation. The method of treating the subterranean formation can include performing enhanced oil recovery (e.g., using the refined beta-glucan as a component of a polymer flooding or sweep fluid), hydraulic fracturing, water shut-off, conformance, or a combination thereof. In a hydraulic fracturing operation, the refined beta-glucan can be used as a component of a fluid for treating the subterranean formation during any suitable stage of the hydraulic fracturing, such as during at least one of a pre-pad stage (e.g., during injection of water with no proppant, and additionally optionally mid- to low-strength acid), a pad stage (e.g., during injection of fluid only with no proppant, such as to begin to break into an area and initiate fractures to produce sufficient penetration and width to allow proppant-laden later stages to enter), or at a slurry stage of the fracturing (e.g., as viscous fluid including proppant).

The method of treating the subterranean formation with the refined beta-glucan can include performing an enhanced oil recovery procedure in the subterranean formation using a liquid that includes the refined beta-glucan. The enhanced oil recovery procedure can include polymer flooding. The method can include using the liquid including the refined beta-glucan in the subterranean formation to sweep petroleum in the subterranean formation toward a well (e.g., a different well from a well the refined beta-glucan was originally placed in). The method can include removing the petroleum from the well (e.g., at least some of the petroleum that was swept toward the well). The liquid that includes the refined beta-glucan can optionally include one or more surfactants (e.g., for surfactant flooding).

### EXAMPLES

Various aspects of the present invention can be better understood by reference to the following Examples which are offered by way of illustration. The present invention is not limited to the Examples given herein.

The term “ambient conditions” as used in the Examples refers to about 18° C. to about 22° C. and about 96 kPa to about 103 kPa. All Examples were performed under ambient conditions unless otherwise indicated.

### Part I. Beta-Glucan Preparation.

### Example I-1

### Beta-Glucan Preparation from Commercial Material

Using a 5000 liter jacketed vessel with moderate agitation, 7 g/L of commercial Actigum® CS6 from Cargill (crude powder blend of scleroglucan and sclerotium rolfsii organism powder) was added to 2400 liters of 11.8° C. water and mixed for 1 hour. After an hour of mixing, the vessel was heated to 85° C. and left under agitation for 12 hours without temperature control. After 12 hours the temperature was 41.3° C. and the vessel was reheated to 80° C. and passed through a Guerin homogenizer at 200 bar of pressure and 300 L/hr.

The homogenized mixture was cooled to 50° C. 4 g/L of CaCl₂*2H₂O was added. pH was reduced to 1.81 using 20% HCl. This mixture was agitated for 30 minutes to enable precipitation of oxalic acid (i.e., as the calcium salt thereof, calcium oxalate).

After maturation, the solution was adjusted back to 5.62 pH using 10% Na₂CO₃ and heated to 85° C. and left under agitation without temperature control for 14 hours, then reheated to 80° C.

After reaching 80° C. 20 g/L of Dicalite 4158 filter aid (water permeability 1.4 Darcy to 3.8 Darcy) was added to the vessel and mixed for 10 minutes.

After mixing, the solution was fed to a clean Choquenet 12 m² press filter with Sefar Fyltris 25080 AM filter cloths at 1400 L/hr recycling the product back to the feed tank for 10 minutes. The pore size of the filter cloths was sufficient to prevent passage of the filter aid. At the end of recycle, the flow was adjusted to 1300 L/hr and passed through the filter. Once the tank was empty an additional 50 liters of water was pushed into the filter. The fluid from this water flush and a 12 bar compression of the cake were both added to the collected permeate. The filter was cleaned after use.

The filtered permeate, water flush, and compression fluid was agitated and heated back to 80° C.

The heated mixture had 6 kg of Dicalite 4158 added thereto and was mixed for 10 minutes. At 140 L/hr this solution was recycled through a clean Choquenet 12 m² press filter with Sefar Fyltris 25080 AM filter cloths at 1400 L/hr for 15 minutes. After the recycle, the tank was passed through the filter at 1400 L/hr.

Without cleaning the filter, 5.33 g/L of Clarcel® DICS (water permeability 2.4 Darcy to 4.0 Darcy) and 6.667 g/L of Clarcel® CBL (water permeability 0.049 Darcy to 0.101 Darcy) were added to the mixture and agitation was performed for one hour while maintaining the temperature at 80° C. This mixture was then recycled through the Dicalite coated Choquenet 12 m² press filter with Sefar Fyltris 25080 AM filter cloths at 1400 L/hr for 15 minutes. After the recycle, the tank was passed through the filter at 1350 L/hr. An additional 50 liters of flush water were pushed through the filter and permeate was collected as well. Compression fluid from the filter was not captured.

This twice filtered material was heated to 85° C. and left agitated without temperature control for 14 hours. At this point the material was reheated to 80° C. for a third filtration step.

The heated mixture had 6 kg of Dicalite 4158 added thereto and mixing was performed for 10 minutes. At 1400 L/hr this solution was recycled through a clean Choquenet 12 m² press filter with Sefar Fyltris 25080 AM filter cloths at 1400 L/hr for 15 minutes. After the recycle, the tank was passed through the filter at 1450 L/hr.

Without cleaning the filter, 5.33 g/L of Clarcel® DICS and 6.667 g/L of Clarcel® CBL were added to the mixture and agitation was performed for one hour while maintaining the temperature at 80° C. This mixture was then recycled through the Dicalite coated Choquenet 12 m² press filter with Sefar Fyltris 25080 AM filter cloths at 1600 L/hr for 15 minutes. After the recycle, the tank was passed through the filter at 1700 L/hr. An additional 50 liters of flush water was pushed through the filter and permeate was collected as well. Compression fluid from the filter was not captured.

The triple filtered permeate was cooled to 60° C. and mixed with 83% IPA at a 1:2 ratio, 2 g IPA solution for each g of scleroglucan solution. This precipitated scleroglucan fibers which can be mechanically separated from the bulk solution. In this example, a tromel separator was used to partition the precipitated fibers from the bulk liquid solution.

After recovery of the fibers they were washed with another 0.5 g 83% IPA solution for each 1 g of initial triple filtered permeate scleroglucan solution.

Wash fibers were dried in an ECI dryer with 95° C. hot water for 1 hour and 13 minutes to produce a product with 88.64% dry matter. This material was ground up and sieved to provide powder smaller in size than 250 microns. The final ground scleroglucan material was Sample 1A, characterized in Part II herein.

A scaled-up version of the procedure was performed, having approximately 100 times the scale of the procedure used to form Sample 1A. The final ground scleroglucan material formed by the scaled-up process was Sample 1B, characterized in Part II herein. The scaled-up version included passing the refined scleroglucan material through several check filters which decreased or minimized the occurrence of small amounts of filter aid in the product but in all other respects seemed to form a product substantially the same as Sample 1A.

### Example I-2

### Beta-Glucan Preparation from Fermentation

Crude Schizophyllan was produced via fermentation using IAM culture collection 9006: C-180. A few grams of material was cultured in multiple steps to generate inoculum for the production fermentation run. Dosing similar nutrients and sugar as the main fermenter, each initial step was run with active oxygen transfer until roughly half the dextrose was consumed. At these small scales, fermentation was more difficult to design and run to precise specifications.

The production fermenter was inoculated with water, nutrients, and substrate as detailed in Table 1. The fermenter was a 15-liter vessel that was 462 mm tall, 202 mm in diameter, and having ellipsoidal heads. To provide mixing, the vessel had an agitator with a Rushton mixing element near the bottom of 128 mm in diameter and two marine agitators higher up that were both 145 mm in diameter. The agitator started at 200 rpm and ramped to 255 rpm over the course of fermentation shown in Table 2. During the fermentation air was supplied at 0.8 VVM (standard volumes of air per volume of liquid per minute) and temperature was controlled to 28° C. Fermentation was stopped after 95 hours with residual dextrose between 1 to 3 g/L. Fermentation ended with some dextrose to avoid unwanted production of enzymes that can consume beta-glucan substrate.

TABLE 1
Production fermenter contents.

| Ingredient | Commercial Product Name | Mass (g) |
|---|---|---|
| Substrate (sugar) | Cargill C*Sweet D 02767 | 470 |
| KH₂PO₄ | KH₂PO₄ | 7 |
| MgSO₄ | MgSO₄ | 10.5 |
| Fava bean flour | CPX55 | 10 |
| Nutrient Blend | Rochette Solulys 048E | 45 |
| | (corn steep water) | |
| Oil | Sunflower Oil | 2.7 |
| AntiFoam | Breviol D102K | 4 |
| Inorganic Nitrogen | NaNO₃ | 45 |
| Water | Water | 9000 |
| Innoculum | Seed train output | 1000 |

TABLE 2
Fermentation characterization.

| | | | Viscosity | BG + | |
|---|---|---|---|---|---|
| | Glucose | | (cP at | biomass | Agitator |
| Hours | (g/L) | pH | 7.3 s⁻¹) | (g/L) | RPM |
| 0 | 26.3 | 4.5 | | | 200 |
| 23 | 21.4 | 4.39 | | | 215 |
| 47 | 13 | 5.33 | 350 | 4.93 | 255 |
| 55 | 11 | 5.45 | 425 | 8.77 | 255 |
| 71 | 5.5 | 5.54 | 1260 | 16.96 | 255 |
| 78.5 | 4.2 | 5.56 | 1320 | 20.16 | 255 |
| 94.5 | 1.6 | 5.66 | 1880 | 27.51 | 255 |

After fermentation was complete, the broth was heat-killed at 95° C. for 5 minutes. The solution was combined while being stirred at 1:1 with 90% IPA (isopropyl alcohol) to precipitate biomass (e.g., a blend of beta-glucan biopolymer and the producing organism). Using cheese cloth to retain fibers, the excess liquid was drained away from the fibers. The fibers were then blended with a 90% IPA that was 50% of the initial fermentation solution volume. Using cheese cloth and 10 bar of pressure, the fibers were drained of liquid as much as possible. They were then dried at 60° C. to 91.2% dry matter (8.8% residual water/IPA). Dried fibers were ground and classified to <500 microns to make the crude schizophyllan powder (Sample C2A in Part II).

To refine the crude material, using a 15-liter jacketed fermenter, 15 g/L of crude schizophyllan was heated to 80° C. for one hour. After heating, the material was fed at 70° C. through a lab homogenizer (APV, Lab 2000 model) at 200-250 bar, dropping to 50° C. during processing. After homogenization, the material was diluted to 8 g/L relative to the original dosing.

The material was then passed through a coarse filtration on a Gautier filter (model ALM 2) covered with 25302 AN membranes and jacketed with 85° C. water to target an 80° C. solution temperature inside the filter. To fit the filter, 1.5 liters of diluted broth was mixed with 72 g of Dicalite 4158 filter aid and heated to 80° C. The pore size of the membranes was sufficient to prevent the filter aid from passing through. The mixture was put into the Gautier filter and 0.1 to 1 barg of pressure was applied, increasing over the filtration to maintain flow at 20-150 mL/min. After 20% of the original diluted broth passes, the filter was opened and the material was put back into the Gautier. At this point, the entire volume was passed through the filter. This filtrate was carried forward to the 2^{nd} filtration step.

The second filtration step used the same filtration equipment setup but with different filter aids. A water mixture of 0.5 liters with 10 grams of Dicalite was run through twice to apply a precoat to the filter. A dose of 5.33 g/L of Clarcel® DICS and 6.667 g/L of Clarcel® CBL was added to the coarse filtrate and agitation was performed for one hour while maintaining temperature at 80° C. This mixture was then added to the Gautier and 20% of the volume was passed. This material was put back in the filter housing. At this point the entire volume was passed through filter and 0.1 to 1 barg of pressure was applied, increasing over the filtration to maintain flow at 20-150 mL/min. This filtrate was carried forward to the 3^{rd} filtration step.

The third filtration was a duplication of the second filtration using the second filtrate instead of the coarse filtrate for feed material. The filtrate from this step was carried forward to alcohol precipitation. When working with larger volumes of broth, the three filtration steps can be run multiple times, blending all of the third filtrate material before precipitation.

To precipitate and dry the material, the third filtrate solution was combined while being stirred at 1:1 with 90% IPA (isopropyl alcohol) to precipitate biopolymer (e.g., biomass that is refined and enriched in the beta-glucan biopolymer fraction). Using cheese cloth to retain fibers, the excess liquid was drained away from fibers. The fibers were then blended with a 90% IPA that was 50% of the initial fermentation solution volume. Using cheese cloth and 10 bar of pressure, the fibers were drained as much as possible of liquid. Afterwards they were dried in a 60° C. to 87.1% dry matter (12.9% residual water/IPA) in an oven (Memmert model ULM 700). Dried fibers were ground and classified to <500 microns to make the beta-glucan material (Sample 2 in Part II).

### Part II. Characterization of Beta-Glucans.

The Samples tested are shown in Table 3. Samples 1A, 1B, and 2 are different inventive examples. Samples C1A, C2A2, C2A, and C2B are Comparative Samples.

TABLE 3
Beta-glucan powder samples.

| Sample | Description |
|---|---|
| 1A | Scleroglucan powder product from Example I-1 |
| 1B | Scleroglucan powder product from scaled-up Example I-1 |
| C1A | Commercially available crude powder blend of scleroglucan |
| | and sclerotium rolfsii organism powder |
| C1B | Commercially available clarified scleroglucan powder |
| 2 | Schizophyllan powder product of Example I-2 |
| C2A | Crude schizophyllan powder from Example I-2. |
| C2B | Schizophyllan powder commercially available from |
| | InvivoGen, San Diego, California |

### Example II-1

### Protein Characterization

A mass balance was used to add 25 mg of the powdered beta-glucan Sample to a beaker. After adding the beta-glucan, 5 mL of deionized water at room temperature was added to the beaker. The solution was then mixed with an IKA® T25 digital Ultra TURRAX® at 20,000 rpm for 8 minutes, forming a single phase with no visible solid particles.

Protein content was measured using a Thermo Fischer Scientific Pierce™ BCA Protein Assay Kit. The colorimetric assay used a set volume (0.1 mL) of a dissolved solution of each beta-glucan Sample in a test tube to measure total protein concentration as compared to protein standards. Each standard and beta-glucan Sample (0.1 mL) were pipetted into appropriately labeled test tubes. A working reagent (2.0 mL), made from 50 parts reagent A and 1 part reagent B, from the Thermo Fischer Scientific Pierce™ BCA Protein Assay Kit, was added to each test tube and the contents were mixed well. The test tubes were covered, shaken to fully mix, and incubated at 60° C. for 30 minutes. The test tubes were cooled to ambient conditions. This material was pipetted onto a microplate and tested with a BioTek® Synergy™ HT spectrophotometer set to 562 nm. Subsequently, the absorbance of each of the Samples were measured within 10 minutes. The average 562 nm absorbance measurement of blank standards were subtracted from the absorbance measurements of the protein standards and the beta-glucan Samples. A standard curve was prepared by plotting the average blank standard-corrected 562 nm measurement for each protein standard versus its concentration in μg/mL. The standard curve was used to determine the protein concentration in each beta-glucan Sample.

Table 4 illustrates the results. The protein concentration in the beta-glucan composition ranged from 7 μg/mL to 348 μg/mL, which corresponded to beta-glucan protein concentrations in the powdered Samples of from about 0.14 wt % to about 6.81 wt % (on a solid basis). Samples 1A and 2 had lower protein content then the Comparative Samples.

TABLE 4
Protein analysis results.

| | | | | | Determined | wt % |
|---|---|---|---|---|---|---|
| | Dry | | Concen- | | value from | protein |
| | Weight | Water | tration | μg/ | Bradford | in sample |
| Sample | (g) | (g) | g/mL | mL | (μg/mL) | (solid/solid) |
| 1A | 0.0201 | 3.9539 | 0.005084 | 5084 | 7.21 | 0.14% |
| C1A | 0.0249 | 4.9180 | 0.005063 | 5063 | 161 | 3.18% |
| C1B | 0.0267 | 5.2449 | 0.005091 | 5091 | 21.0 | 0.41% |
| 2 | 0.0236 | 4.6552 | 0.005070 | 5070 | 20.4 | 0.40% |
| C2A | 0.0254 | 4.9768 | 0.005104 | 5104 | 348 | 6.81% |
| C2B | 0.0226 | 4.5629 | 0.004953 | 4953 | 147 | 2.97% |

### Example II-2

### Obscuration Characterization

A mass balance was used to add 200 mg of powdered beta-glucan Sample to a small beaker. After adding beta-glucan, 20 mL of deionized water at room temperature was added to the beaker. The solution was then mixed with an IKA® T25 digital Ultra TURRAX® at 20,000 rpm for 8 minutes. Glutaraldehyde (900 ppm) was added as a biocide to the Samples. Samples were filtered through a Pall Acrodisc® 37 mm diameter syringe filter with a 1 μm pore size glass filter membrane. The filtered Samples were then diluted to 1:9 by volume beta-glucan:DI water (for a final concentration of 1 mg/mL). The Samples were placed in a Malvern Mastersizer 3000, analyzing transmission at 633 nm and 470 nm, which reported obscuration of the Samples (i.e., 1/transmittance).

The results are illustrated in Table 5-A, showing Samples 1A, 1B, and 2 to be in the range of 0.02% to 0.46% obscuration, compared to 2.05 to 17.3 for the Comparative Samples. A small amount of residual filter aid may have been present in Samples 1A and 2 that impacted the obscuration values.

TABLE 5-A
Obscuration of Samples.

| Sample | Concentration (g/L) | Obscuration (%) |
|---|---|---|
| 1A | 1.0 | 0.31 |
| 1B | 1.0 | 0.02 |
| C1A | 1.0 | 17.3 |
| C1B | 1.0 | 2.05 |
| 2 | 1.0 | 0.46 |
| C2A | 1.0 | 6.36 |

### Example II-3

### Dynamic Mechanical Analysis (DMA) Characterization

Viscoelastic behavior was measured using a TA Instruments Q800 Dynamic Mechanical Analyzer. The powdered Samples were loaded into a 35 mm dual cantilever with powder cell. The Samples were sprinkled into the lower basin of the powder cell. The mass and volume were not controlled, such that the relative magnitude of the signals collected (modulus, tan delta) between different Samples did not provide a quantitative comparison; however, the relationship to temperature was quantitative. The Samples were equilibrated at −20° C. for 10 minutes. The temperature was ramped at 3° C./min to 250° C. Oscillation conditions: strain was 15 μm; frequency was 10 Hz.

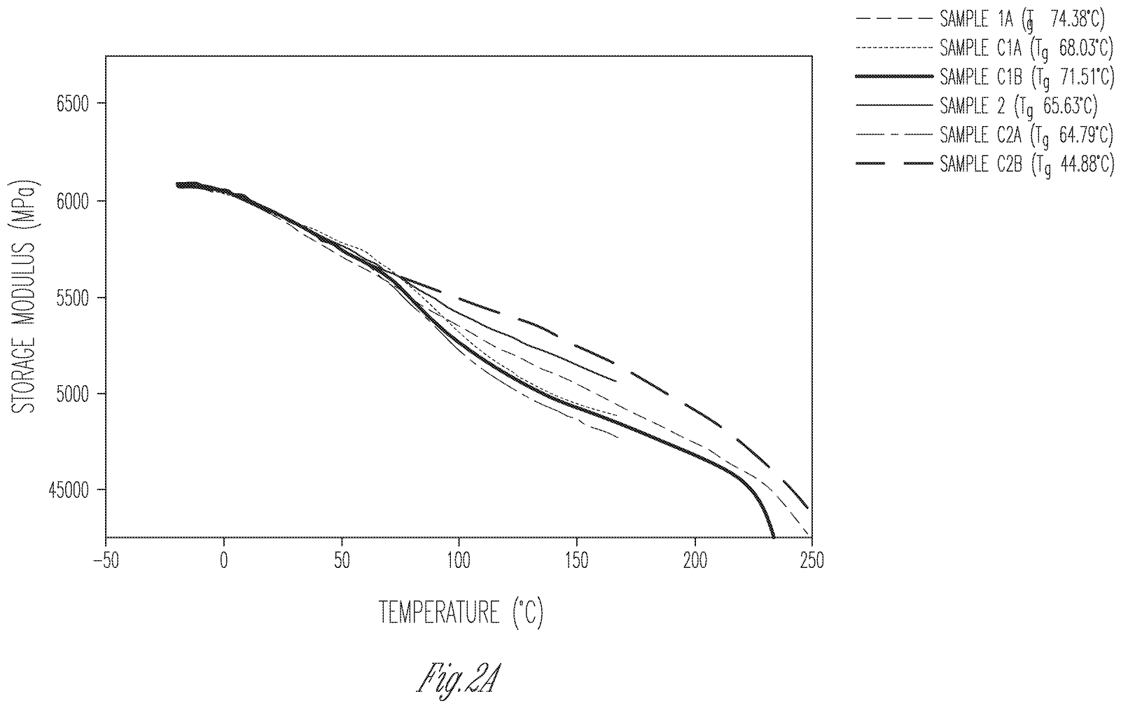

The results are shown in FIGS. 2A-B. FIG. 2A illustrates storage modulus versus temperature. FIG. 2B illustrates tan delta versus temperature. For the scleroglucan Samples the temperature trend for T_{g} as measured by onset of storage modulus change as detected by dynamic mechanical analysis and peak tan delta was Sample C1A<Sample C1B<Sample 1A. Likewise, the schizophyllan Sample 2 had a higher temperature for both the T_{g} as measured by onset of storage modulus change and peak tan delta as compared to Comparative Samples C2A and C2B. The curves were empirically shifted vertically to gauge relative amounts of modulus drop after going through a transition. The magnitude of the modulus drop after the T_{g} transition relates to amount of cross-linking and crystallinity present. The more cross-linking in the sample, the less of a modulus drop is recorded. Schizophyllan Samples 2, C2A, and C2B showed earlier onsets than the other treatments, which could be due to, for example, moisture content (e.g., more water present could plasticize and shift a glass transition temperature lower), lower molecular weight, or a combination thereof.

### Example II-4

### Atomic-Force Microscopy (AFM) and Confocal Laser Scanning Microscopy (CLSM) Characterization

### Atomic-Force Microscopy (AFM).

A mass balance was used to add 25 mg of powdered beta-glucan Sample to a small beaker. After adding the beta-glucan, 5 mL of deionized water at room temperature was added to the beaker. The solution was then mixed with an IKA® T25 digital Ultra TURRAX® at 20,000 rpm for 8 minutes, at which point the solution was a single phase with no visible solid particles.

The single phase Samples were diluted with DI water down to a beta-glucan concentration of about 5 μg/mL. A thin layer (about 50 μL) of the solution was placed on freshly cleaved mica surface and dried in air.

AFM imaging was performed in desiccated air (relative humidity less than about 1%) using a silicon tip/cantilever (with uncoated backside) having nominal spring constant k=2 N/m and a manufacturer's estimated tip radius of curvature of R=6 nm. A Keysight 5500 scanning probe microscope and WITec Digital Pulsed Force mode add-on (intermittent quasistatic contact modality) were employed for all images. The scanning rate was 1 line/second for 2×2 micron images (512×512 pixels) and 0.5 lines/second for 10×10 micron images (1024×1024 pixels).

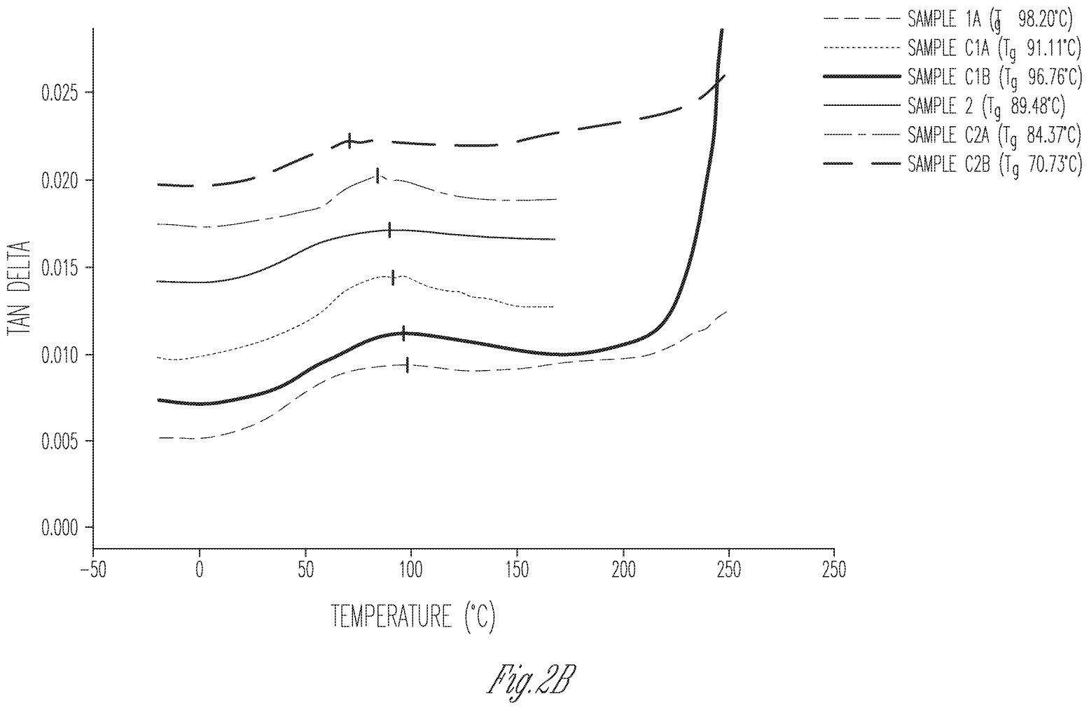

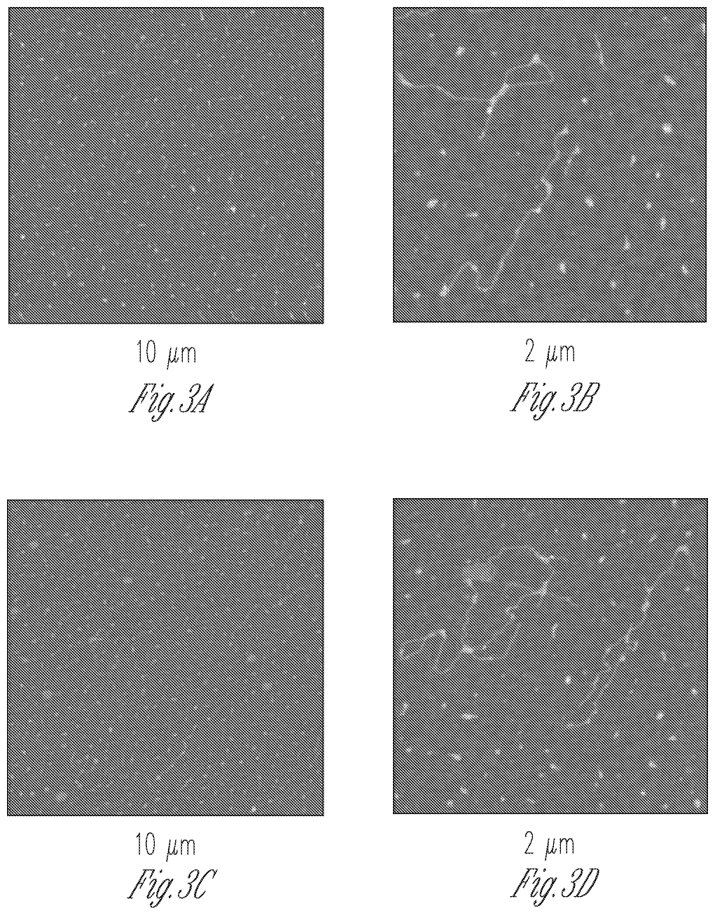

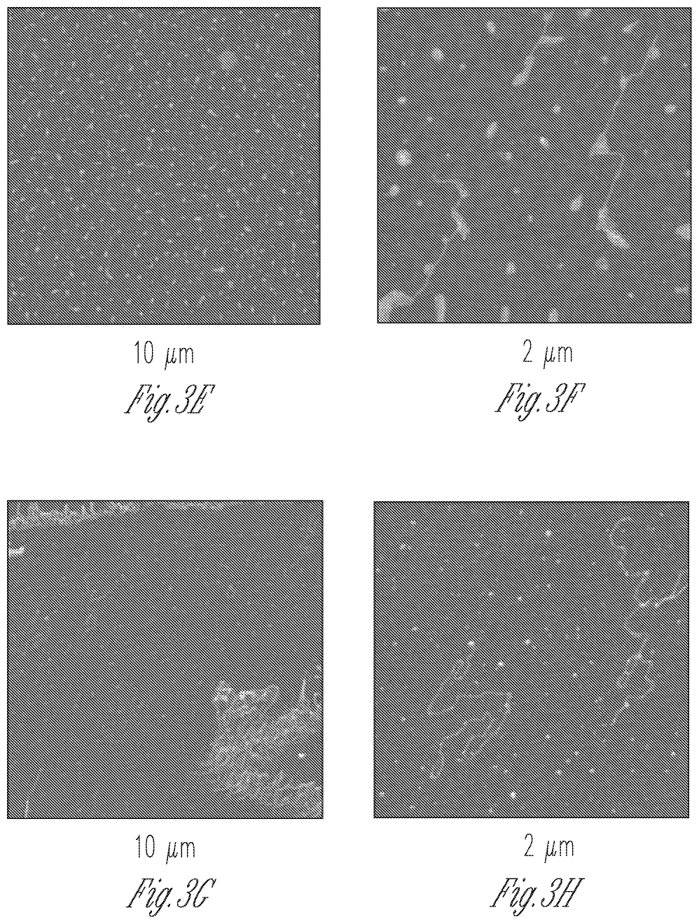

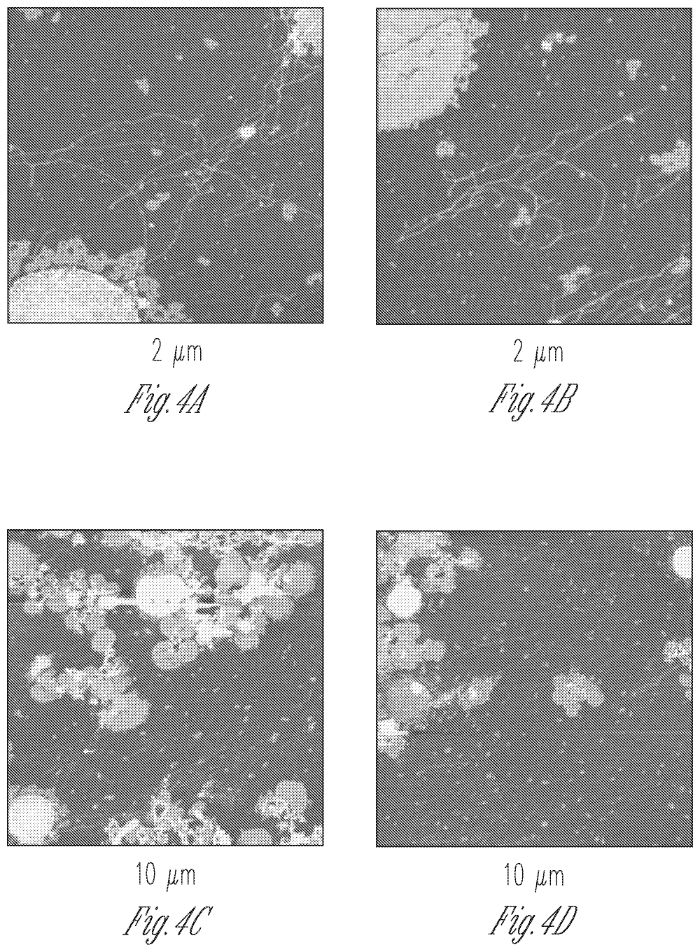

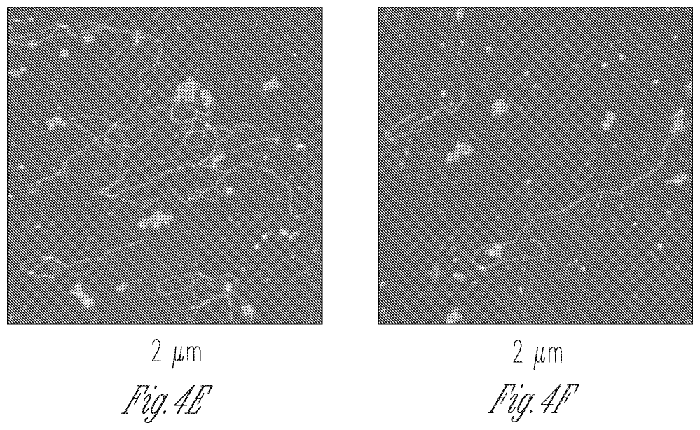

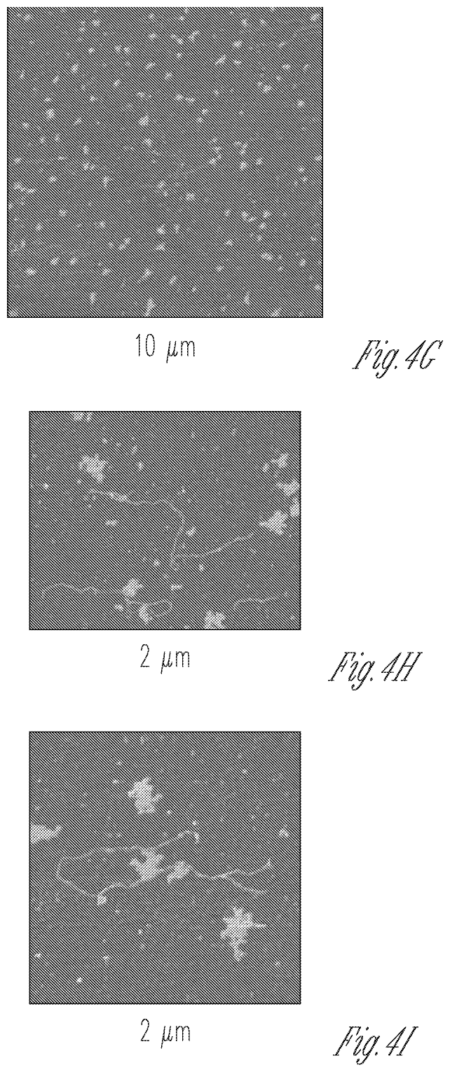

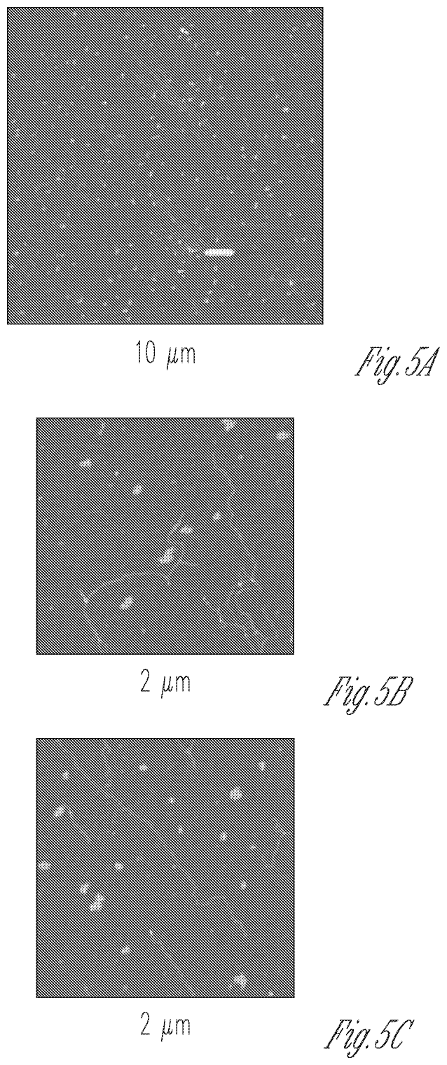

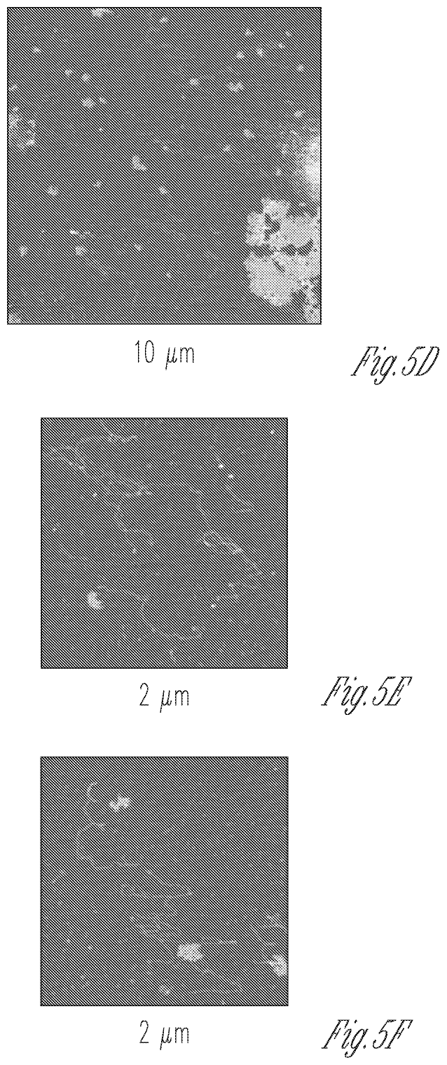

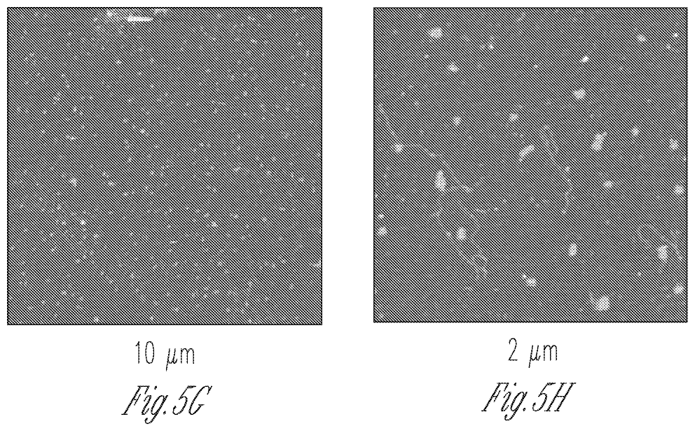

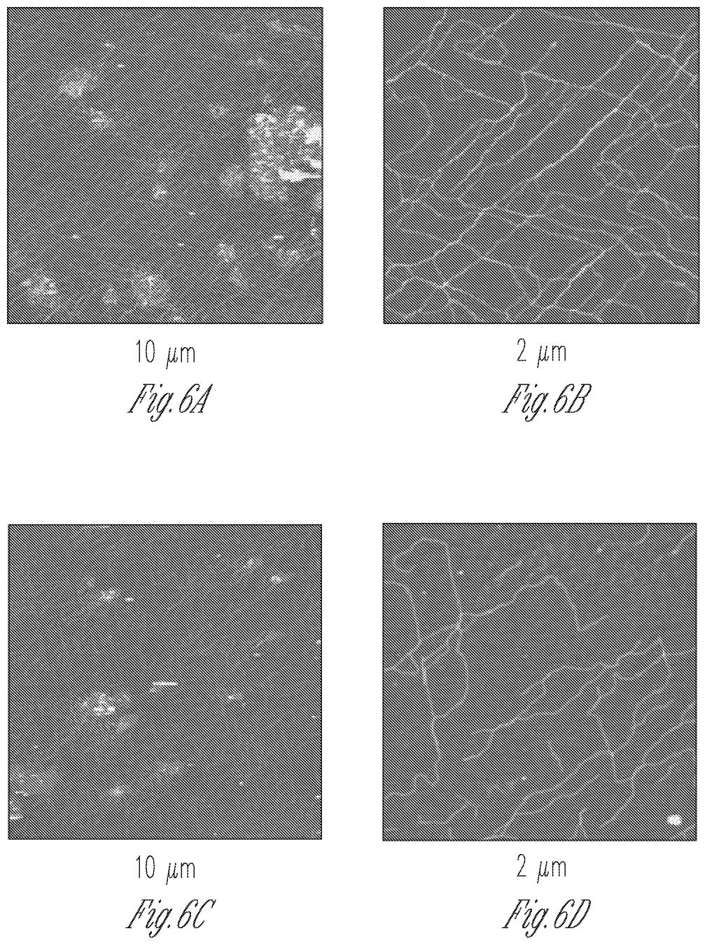

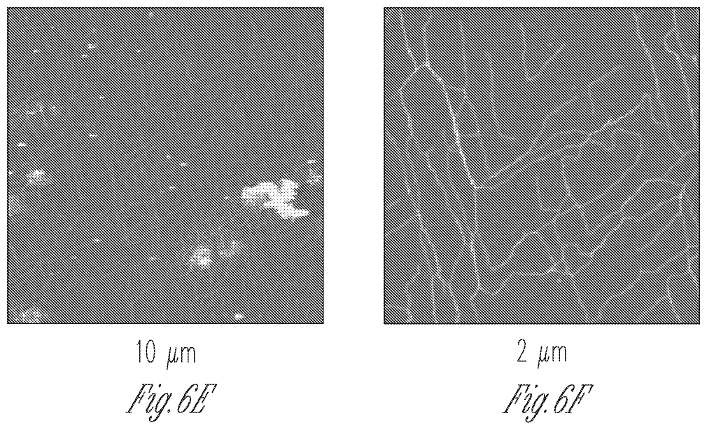

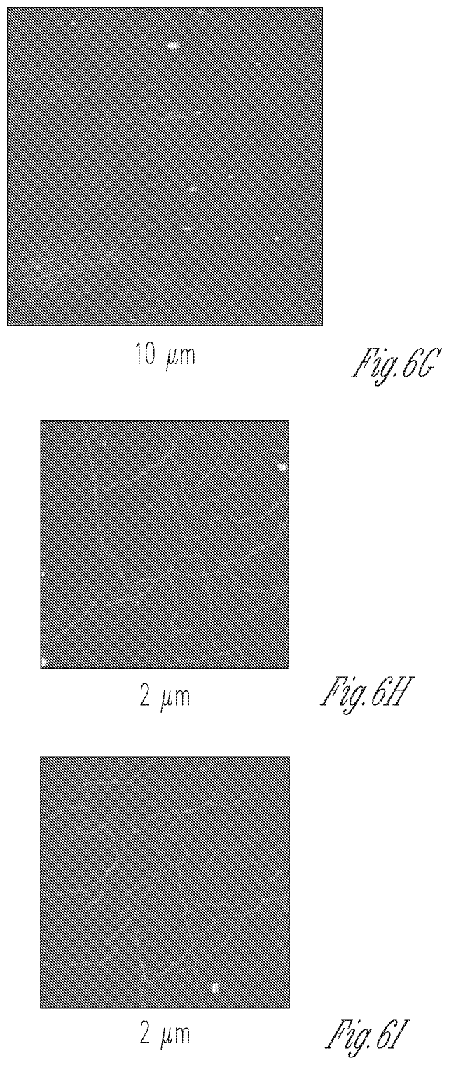

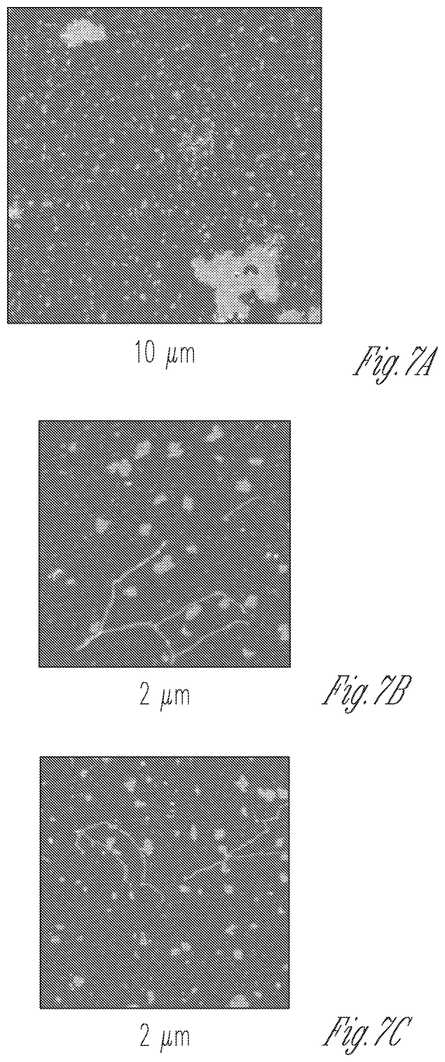

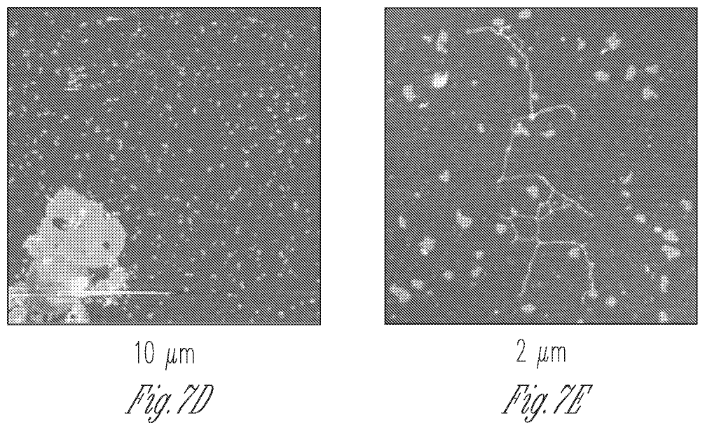

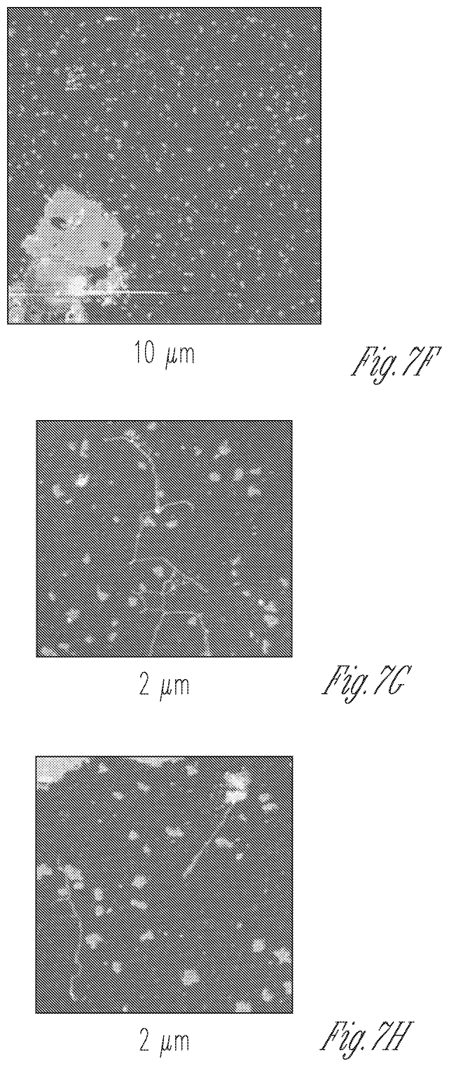

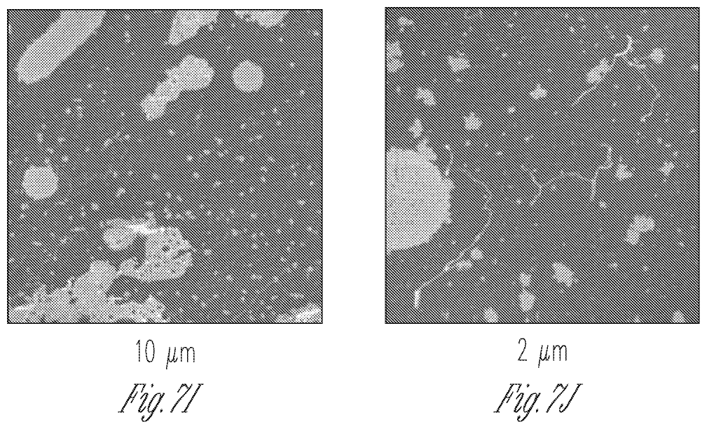

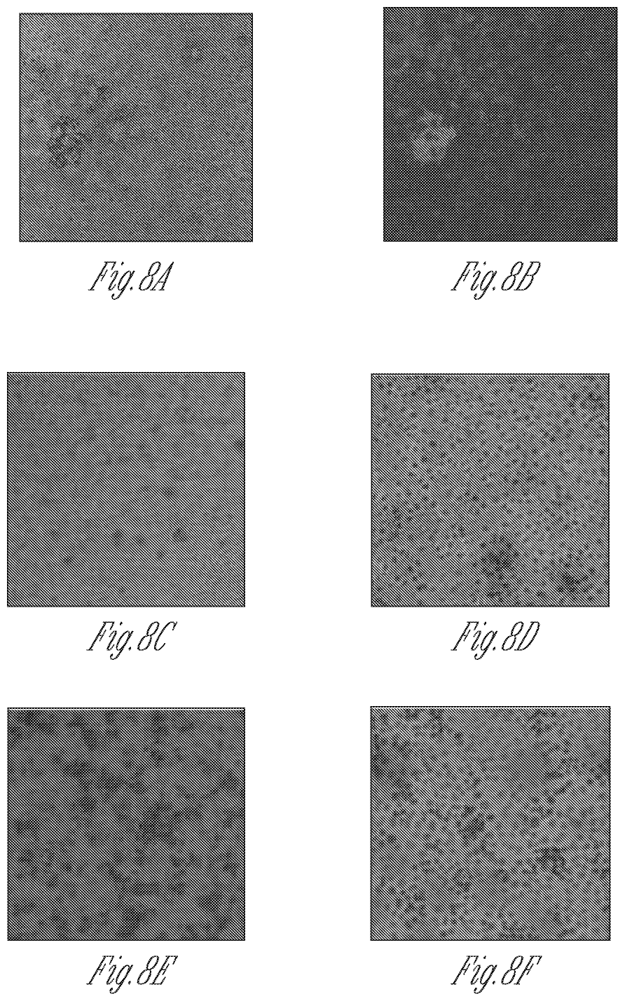

The AFM results are shown in FIGS. 3A-H, 4A-I, 5A-H, 6A-J, and 7A-I, as shown in Table 6. FIGS. 3A-H illustrate AFM images of Sample 1A at 2 micron and 10 micron image size. FIGS. 4A-I illustrate AFM images of Sample C1A at 2 micron and 10 micron image size. FIGS. 5A-H illustrate AFM images of Sample C1B at 2 micron and 10 micron image size. FIGS. 6A-I illustrate AFM images of Sample 2 at 2 micron and 10 micron image size. FIGS. 7A-J illustrate AFM images of Sample C2A, at 2 micron and 10 micron image size. Visual differences can be seen between the images for Sample 1A as compared to Sample C1A and Sample C1B. The uniformity in size and shape of the generally round light domains in the Sample 1A images is in contrast to the large domains of Sample C1A and Sample C1B which possibly correspond to microgel formations. The filament structures also appear different between Sample 1A as compared to Sample C1A and Sample C1B. Some of the domains appear to contain ordered filaments and some random strands.

TABLE 6
AFM images.

| FIG. | Sample | Size of image (microns) |
|---|---|---|
| 3A | 1A | 10 × 10 |
| 3B | 1A | 2 × 2 |
| 3C | 1A | 10 × 10 |
| 3D | 1A | 2 × 2 |
| 3E | 1A | 10 × 10 |
| 3F | 1A | 2 × 2 |
| 3G | 1A | 10 × 10 |
| 3H | 1A | 2 × 2 |
| 4A | C1A | 2 × 2 |
| 4B | C1A | 2 × 2 |
| 4C | C1A | 10 × 10 |
| 4D | C1A | 10 × 10 |
| 4E | C1A | 2 × 2 |
| 4F | C1A | 2 × 2 |
| 4G | C1A | 10 × 10 |
| 4H | C1A | 2 × 2 |
| 4I | C1A | 2 × 2 |
| 5A | C1B | 10 × 10 |
| 5B | C1B | 2 × 2 |
| 5C | C1B | 2 × 2 |
| 5D | C1B | 10 × 10 |
| 5E | C1B | 2 × 2 |
| 5F | C1B | 2 × 2 |
| 5G | C1B | 10 × 10 |
| 5H | C1B | 2 × 2 |
| 6A | 2 | 10 × 10 |
| 6B | 2 | 2 × 2 |
| 6C | 2 | 10 × 10 |
| 6D | 2 | 2 × 2 |
| 6E | 2 | 10 × 10 |
| 6F | 2 | 2 × 2 |
| 6G | 2 | 10 × 10 |
| 6H | 2 | 2 × 2 |
| 6I | 2 | 2 × 2 |
| 7A | C2A | 10 × 10 |
| 7B | C2A | 2 × 2 |
| 7C | C2A | 2 × 2 |
| 7D | C2A | 10 × 10 |
| 7E | C2A | 2 × 2 |
| 7F | C2A | 10 × 10 |
| 7G | C2A | 2 × 2 |
| 7H | C2A | 2 × 2 |
| 7I | C2A | 10 × 10 |
| 7J | C2A | 2 × 2 |

### Confocal Laser Scanning Microscopy (CLSM).

A mass balance was used to add 25 mg of powdered beta-glucan Sample to a small beaker. After adding the beta-glucan, 5 mL of deionized water at room temperature was added to the beaker. The solution was then mixed with an IKA® T25 digital Ultra TURRAX® at 20,000 rpm for 8 minutes, at which point the solution is a single phase with no visible solid particles. Glutaraldehyde (biocide) was added to the Samples in an amount of 900 ppm.

The Samples, filtered through a Pall Acrodisc® 37 mm diameter syringe filter with a 5 micron pore size glass filter membrane, were diluted down to a beta-glucan concentration of about 5 μg/mL. To some Samples, one drop (about 0.3 g) of Congo Red (for carbohydrates) was added to 2 mL of the Sample solution for staining. To some Samples, one drop (about 0.3 g) of Fast Green FCF (for protein) were added into 2 mL of the Sample solution for staining. A thin layer (about 50 μL) of the solution was placed on a glass surface and dried in air.

The glass surface including the Sample was loaded and images were acquired using a Leica Microsystems TCS SP8 Confocal Laser Scanning Microscope equipped with 4 excitation lasers, 2 photomultiplier tubes (PMT), and one Leica HyD detector. Excitations were set at 532 nm (for Congo red) and 638 nm (for Fast green FCF), and emissions were collected between 550-675 nm and 680-750 nm, respectively. A 40× air objective (numerical aperture (NA)=0.65) was used. Each image contained 2048×2048 pixels and was acquired with line average of 16 and frame average of 4. The color green in the images corresponds to carbohydrates and the color red corresponds to protein.

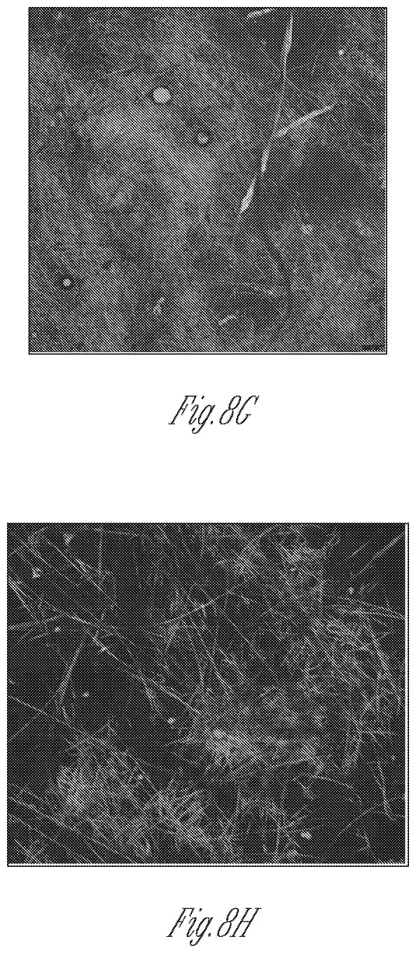

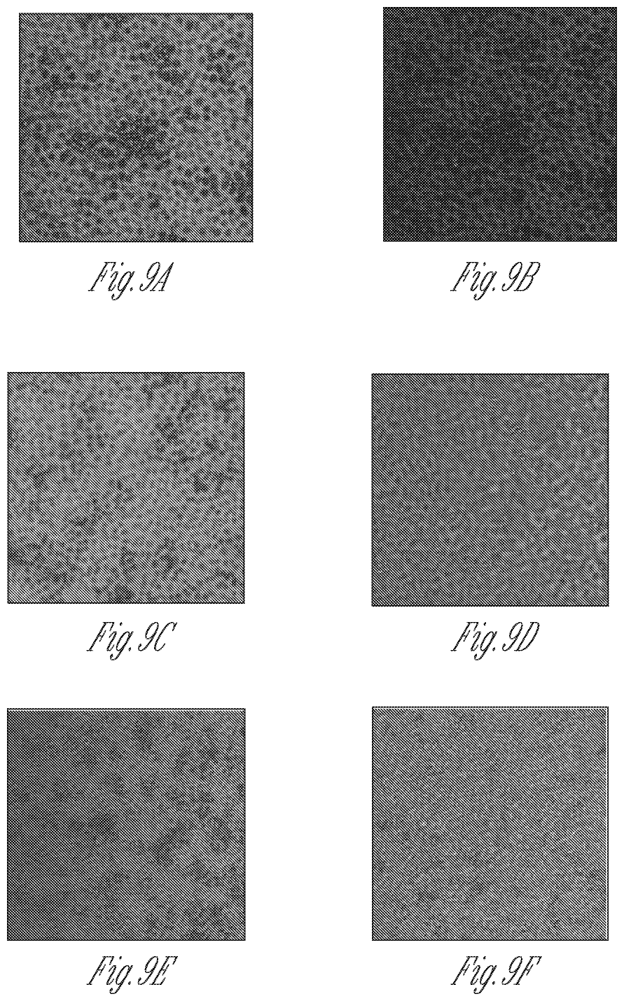

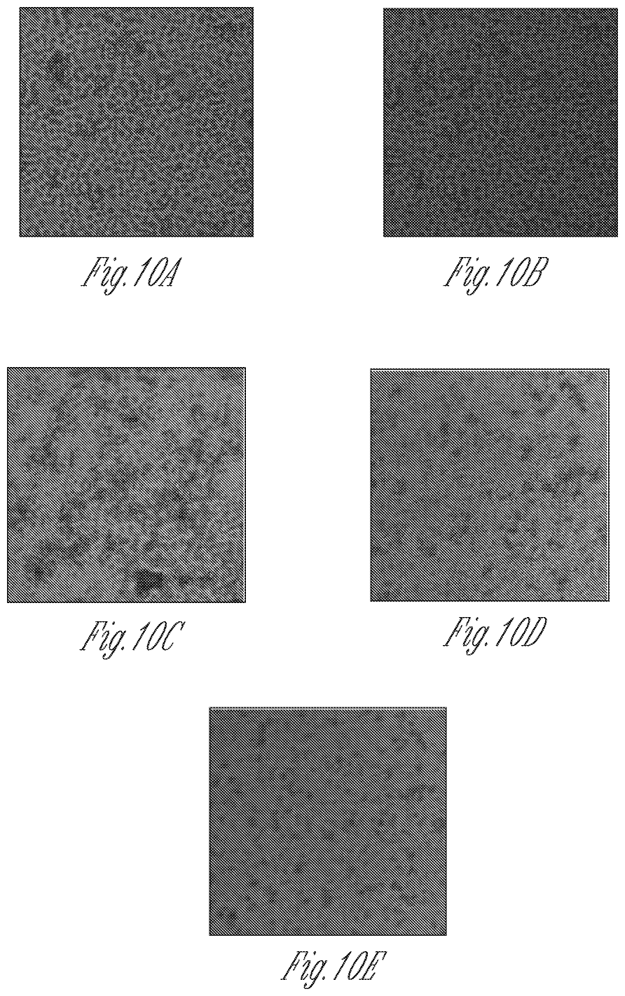

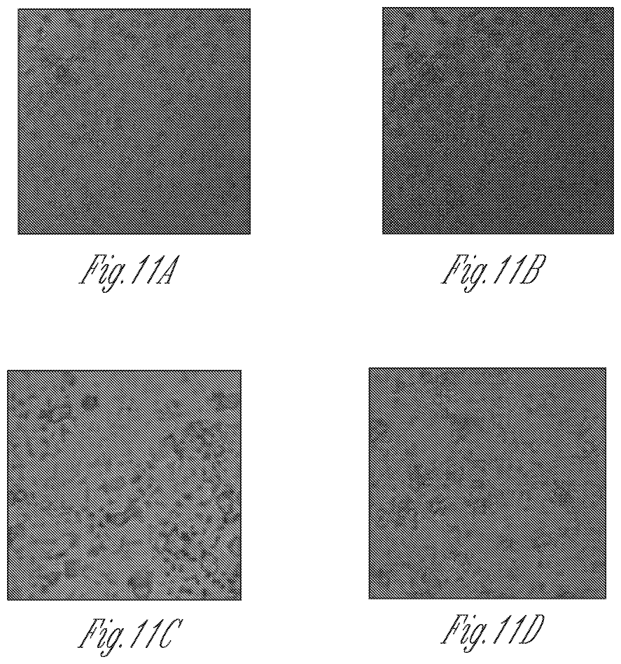

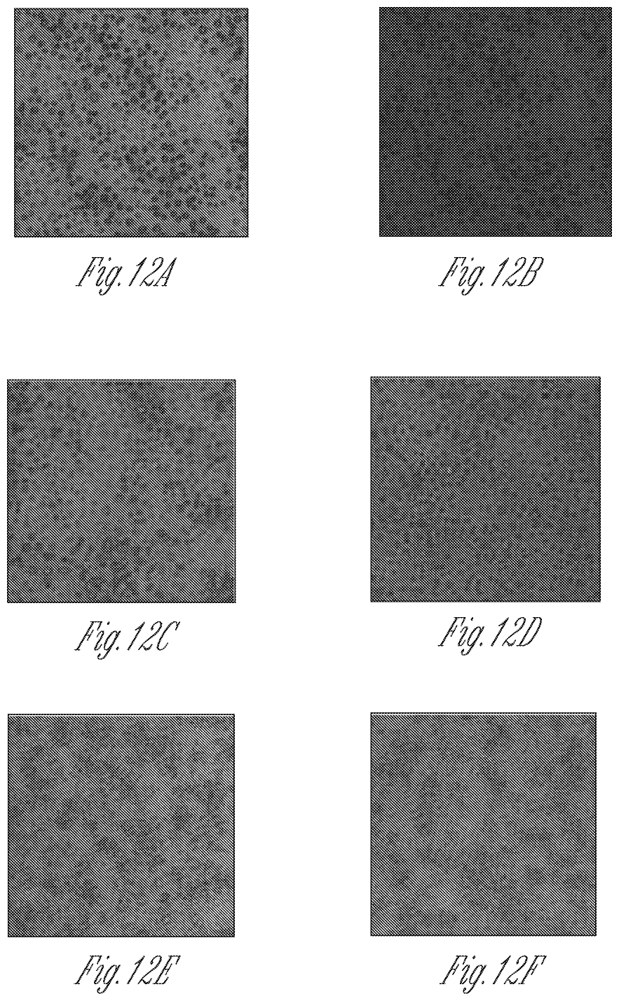

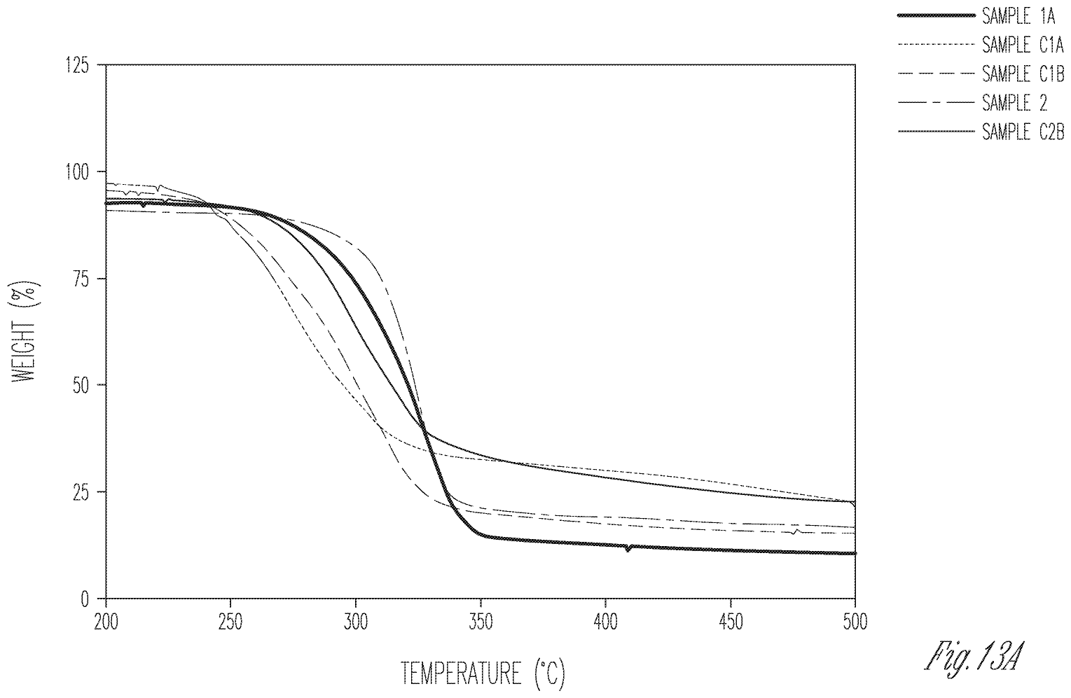

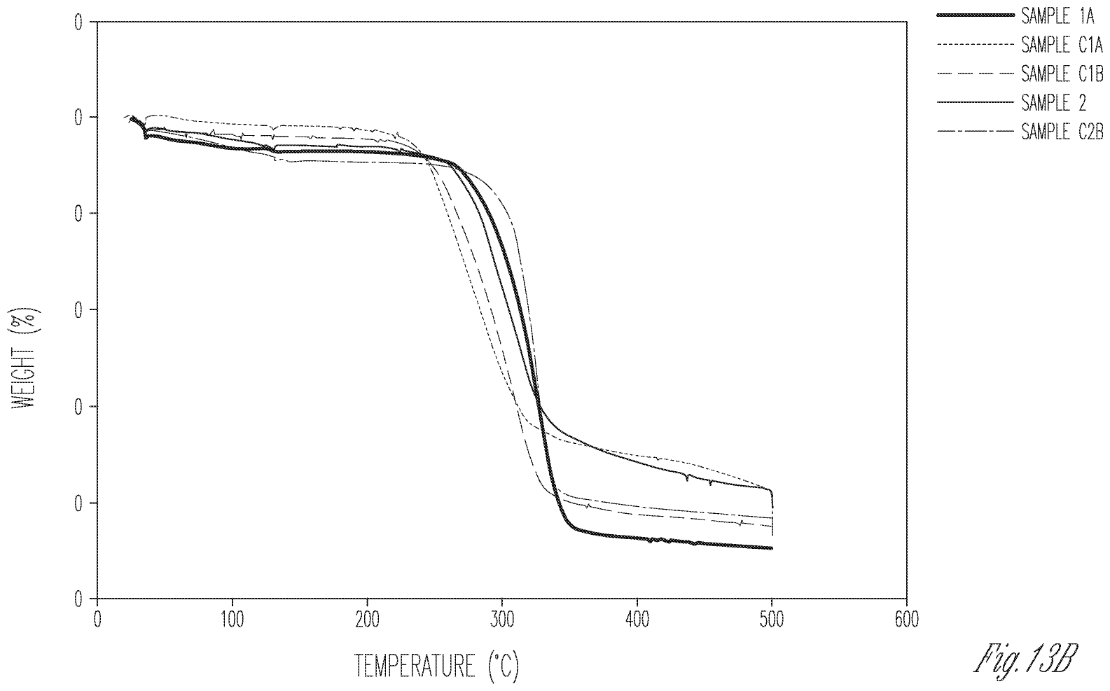

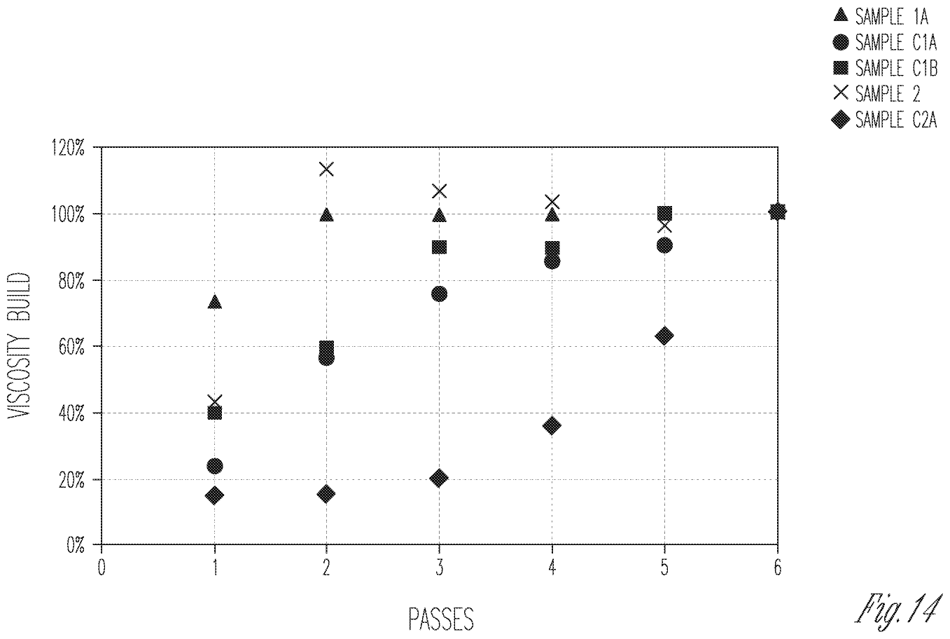

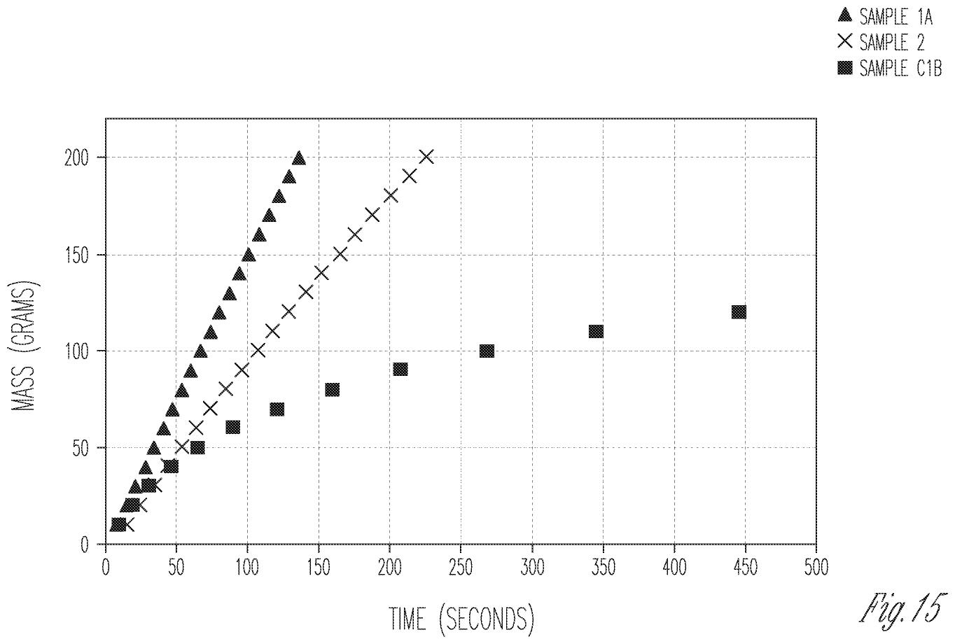

The CLSM results are shown in FIGS. 8A-H, 9A-F, 10A-E, 11A-D, and 12A-F as shown in Table 7. All images were gathered at 40× magnification. FIGS. 8A-F illustrate CLSM images of Sample 1A. FIGS. 8G-H illustrate CLSM images of Sample 1B. FIGS. 9A-F illustrate CLSM images of Sample C1A. FIGS. 10A-E illustrate CLSM images of Sample C2A. FIGS. 11A-D illustrate CLSM images of Sample 2. FIGS. 12A-F illustrate CLSM images of C2A. The carbohydrates form a continuous matrix on the surface, with small embedded domains (about 1-2 μm in size); some large domains (>15 μm) were present as well. This aligned with AFM images. Protein seems to coexist with carbohydrates, with higher concentrations associated with larger carbohydrate domains. Some differences were observed among Samples. It is not clear how the dye crystals affected the observed structures.

TABLE 7
CLSM images.

| FIG. | Sample | Protein/carbohydrate |
|---|---|---|
| 8A | 1A | Carbohydrate |
| 8B | 1A | Protein |
| 8C | 1A | Carbohydrate |
| 8D | 1A | Carbohydrate |
| 8E | 1A | Carbohydrate |
| 8F | 1A | Carbohydrate |
| 8G | 1B | Carbohydrate |
| 8H | 1B | Protein |
| 9A | C1A | Carbohydrate |
| 9B | C1A | Protein |
| 9C | C1A | Carbohydrate |
| 9D | C1A | Carbohydrate |
| 9E | C1A | Carbohydrate |
| 9F | C1A | Carbohydrate |
| 10A | C2A | Carbohydrate |
| 10B | C2A | Protein |
| 10C | C2A | Carbohydrate |
| 10D | C2A | Carbohydrate |
| 10E | C2A | Carbohydrate |
| 11A | 2 | Carbohydrate |
| 11B | 2 | Protein |
| 11C | 2 | Carbohydrate |
| 11D | 2 | Carbohydrate |
| 12A | C2A | Carbohydrate |
| 12B | C2A | Protein |
| 12C | C2A | Carbohydrate |
| 12D | C2A | Carbohydrate |
| 12E | C2A | Carbohydrate |
| 12F | C2A | Carbohydrate |

### Example II-5

### Thermogravimetric Analysis (TGA)

A small amount (about 2 mg) of the powdered Sample was loaded on a sample plate. The Sample was placed in a TA Instruments Q50 TGA for analysis, which ran Samples in the presence of nitrogen gas at a flow rate of 100 mL/min. The TGA furnace was less than 35° C. before putting the Sample into it. The TGA was equilibrated at 35 C and was held at 35° C. for 5 minutes. The Sample was ramped up to 130° C. at 20° C./minute. The Sample was held at 130° C. for 20 minutes, to ensure water and volatiles were eliminated before tracking the decomposition. The Sample was ramped up to 500° C. at 20° C./minute. The Sample was held at 500° C. for 10 minutes.

The results are shown in FIGS. 13A-B, each showing weight % versus temperature, with FIG. 13B showing a zoomed-in temperature scale. In the temperate range from 200° C. to 400° C., the Samples C1A and C1B showed weight loss and decomposition at lower temperatures as compared to Samples 1A, 2, and C2A. Samples 1A and 2 had the highest weight loss and majority decomposition temperatures.

### Example II-6

### Moisture Analysis—Desiccation by Thermogravitmetry Filterability

Samples of Sample 1A and 1B were prepared having different moisture content by drying for different lengths of time. The apparatus was turned on and opened. The aluminum dish was placed on its support in the measuring chamber. The analyzer was closed so the apparatus could tare the dish. The analyzer was opened, and 1-2 g of sample was uniformly distributed in the aluminum dish, and the analyzer was closed. The measurement function was engaged, and the % dry matter was read using a Mettler-Toledo LP 16 Thermogravimetric Analyzer operating at 100° C. set point. The system monitored weight loss and recorded final wt % dry matter value after 2 minutes of stable weight measurement.

Filterability Ratio determination. At ambient conditions, water (250 mL) was added to a VWR1213-1173 Borosilicate glass 3.3 400 mL beaker. While agitating the water using an IKA RW20 DZM stirrer at 700 rpm, Sample 1A (0.25 g+/−0.1 g) was sprinkled into the beaker onto the wall of the vortex. During the agitation, the center of the bottom of the shaft was located 2 cm above the center of the bottom of the beaker. The contents of the beaker were agitated for 20 minutes to incorporate the solids and build some viscosity before increasing agitation. The agitation was increased to 2,000 rpm, and agitation was performed for 4 hours.

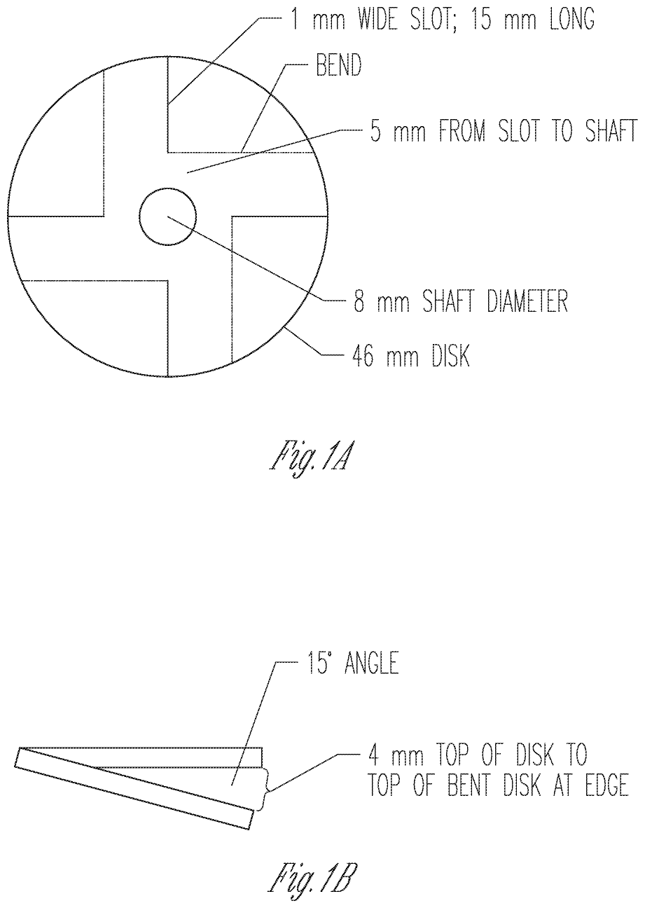

The shaft and mixing element used on the IKA RW20 DZM stirrer is shown in FIGS. 1A-B and have the following geometry. An 8 mm diameter shaft has a 46 mm diameter disc 1 mm thick welded to the bottom of shaft. The disc has four 1 mm slots cut at 90 degrees from each other. They extend from the exterior of the disc to within 5 mm of the end of the shaft. In the clockwise direction the side of the slot on the disk is bent downwards 4 mm (as measured from the top of the disc to top of the disk at the outer edge of disk) with the fold making a right angle with the slot and commencing at the base of the slot and extending to the edge of the disc. The descent angle at the fold is about 15 degrees. FIG. 1A illustrates atop view of the stirrer, FIG. 1B illustrates a side view of one of the four bends of the stirrer, as viewed perpendicularly to one the slot adjacent to the bend.

The subsequent filtration testing is carried out within one hour of solubilization before any microbe formation in the solution can negatively impact the Filterability Ratio. A Pall stainless steel filter housing (4280) was assembled with a 47 mm diameter Millipore AP25 filter (AP2504700), having a pore size of 2 microns. For each Sample, the dispersion was passed through the housing using a flow rate of 100-300 ml/min, and the filtered dispersion was used for future steps. The Pall stainless steel filter housing (4280) was assembled with 47 mm diameter, 1.2 μm pore size, EMD Millipore mixed cellulose esters filter (part #RAWP04700), with >200 mL of solution. A container was placed on a mass balance for recording mass of material passing through the filter. Pressure was applied to the filter. The filter was unplugged and pressure was adjusted to achieve a target flux of 1-3 g/s. Once target flux was established, a constant pressure was maintained and the time needed to filter 60 g, 80 g, 160 g, and 180 g of solution through the filter was measured. Filterability Ratio was determined as (time(180 g)−time (160 g))/(time(80 g)−time (60 g)). The elapsed time between the assembly of the Pall stainless steel filter with >200 mL of solution and the time to complete the passing of the 180 g solution through the filter took between 30 minutes and 4 hours. Results of the moisture content analysis and Filterability Ratio determination are shown in Table 8. The Sample having 98.7% dry matter, dispersed according to the procedure described herein, had a poor Filterability Ratio. Sample 1B had a bulk density of 0.402 kg/L, which was determined by weighing a volume of about 200 mL of the powdered Sample without shaking or tapping it down, and then calculating the weight per volume.

TABLE 8
Moisture content and Filterability Ratio for Sample 1A
and 1B materials having various moisture content.

| Sample | Dry Matter (wt %) | Filterability Ratio |
|---|---|---|
| 1A | 88.0% | 1.35 |
| 1A | 90.5% | 1.29 |
| 1B | 91.0% | 1.06* |
| 1A | 92.5% | 1.26 |
| 1A | 94.5% | 2.1 |
| 1A | 98.7% | Blocked <100 ml |

*measured after 12 passes through the Magic Lab at 20,000 rpm

### Example II-7

### Viscosity Build and Filterability

Scleroglucan Samples were sprinkled onto the vortex in a stirred beaker and mixed for 5 minutes to form a 2 g/L solubilized solutions of each Sample. The solutions were placed in an IKA® Magic Lab® in UTL configuration with a 4M rotor stator pair running unit at 26,000 rpm. The IKA® Magic Lab® is an inline mixer using a rotor stator to impart shear on the solution. As used herein, one “pass” through the Magic Lab denotes feeding solution to the Magic Lab and collecting it at the discharge, wherein the solution has been processed through the equipment one time. Each pass through the single rotor stator assembly of the Magic Lab subjected the Sample to a shear rate (s⁻¹) of about 10 times the rotor speed setting in rpm for a duration of about 0.01 s to about 1 s. After the pass through the Magic Lab, a portion of each solution (50 mL) was set aside. The remainder of each Sample was passed through the Magic Lab using the same setting and equipment. After the second pass, another portion of each solution (50 mL) was set aside. This process was repeated with a total of 6 passes through the Magic Lab. The viscosity of the Samples was measured using a Brookfield DV2T (Spindle 21) viscometer. “Viscosity Build” was calculated for the Samples, defined as the ratio of viscosity measured after a pass through the Magic Lab divided by the ultimate viscosity, or viscosity measured after 6 passes of solubilization. The results are presented in Table 9, and FIG. 14 which illustrates Viscosity Build versus passes through the Magic Lab. Samples 1A, 1B, and 2 had more rapid viscosity build than the Comparative Samples. A rapid build of viscosity up to 90% of ultimate viscosity occurred for Samples 1A, 1B, and 2 in only two passes, compared to more passes required for the Comparative Samples. An rpm of 1 rpm is 1/60 s⁻¹ or 0.0167 s⁻¹, giving 12 rpm=0.2 s⁻¹, 30 rpm=0.5 s⁻¹, and 60 rpm=1 s⁻¹.

TABLE 9
Viscosity Build of solubilized samples in the Magic Lab.

| | | Average | Viscosity | Viscosity | Viscosity |
|---|---|---|---|---|---|
| | | Viscosity | Build at 12 | Build at 30 | Build at 60 |
| Sample | Pass | Build | rpm | rpm | rpm |
| 1A | 1 | 73% | 70% | 73% | 75% |
| 1A | 2 | 100% | 100% | 100% | 100% |
| 1A | 3 | 98% | 100% | 100% | 95% |
| 1A | 6 | 100% | 100% | 100% | 100% |
| 1B* | 1 | 62% | 64% | 57% | 66% |
| 1B* | 2 | 98% | 98% | 96% | 100% |
| 1B* | 3 | 101% | 100% | 103% | 101% |
| 1B* | 6 | 100% | 100% | 100% | 100% |
| C1A | 1 | 25% | 70% | 24% | 31% |
| C1A | 2 | 57% | 53% | 57% | 62% |
| C1A | 3 | 75% | 73% | 76% | 77% |
| C1A | 6 | 100% | 100% | 100% | 100% |
| C1B | 1 | 40% | 33% | 40% | 46% |
| C1B | 2 | 60% | 50% | 60% | 69% |
| C1B | 3 | 92% | 100% | 90% | 85% |
| C1B | 6 | 100% | 100% | 100% | 100% |
| 2 | 1 | 44% | 33% | 43% | 54% |
| 2 | 2 | 122% | 142% | 113% | 110% |
| 2 | 3 | 110% | 117% | 107% | 108% |
| 2 | 6 | 100% | 100% | 100% | 100% |
| C2A | 1 | 17% | 11% | 15% | 24% |
| C2A | 2 | 18% | 10% | 16% | 27% |
| C2A | 3 | 25% | 19% | 21% | 34% |
| C2A | 6 | 100% | 100% | 100% | 100% |

*measured using 20,000 rpm passes through the Magic Lab instead of 26,000 rpm.

Table 10 shows the Filterability Ratio of the solubilized Sample 1A, as tested after each pass through the Magic Lab, demonstrating that the filterability remained relatively constant. Table 10 also shows the Filterability Ratio of solubilized Sample 1B, after 12 passes at 20,000 rpm each. Similarly, the solubilized Sample 2 demonstrated good filterability after 6 passes, having a Filterability Ratio of 1.2, based on 25 seconds to pass 160 g to 180 g and 21 seconds to pass 60 g to 80 g of material. FIG. 15 illustrates mass of filtrate versus time for the solubilized Sample 1, Sample 2, and Sample C1B. The solubilized Sample C1A plugged the pre-filter before passing 200 g of filtrate. Solubilized Sample C1B plugged the 1.2 micron filter before passing 180 g. Because the solubilized Samples C1A and C1B plugged the pre-filter and filter, the Filterability Ratio could not be quantified; however, if a filterability ratio was quantified it would exceed 1.5.

TABLE 10
Filterability Ratio of solubilized Samples 1A and 1B
after passes through the Magic Lab.

| | | Time (s) | | | | Filterability |
|---|---|---|---|---|---|---|
| Sample | Pass | 60 g | 80 g | 160 g | 180 g | Ratio |
| 1A | 1 | 69 | 97 | 225 | 260 | 1.25 |
| 1A | 2 | 58 | 78 | 164 | 187 | 1.15 |
| 1A | 3 | 61 | 81 | 170 | 194 | 1.2 |
| 1A | 4 | 46 | 61 | 128 | 146 | 1.2 |
| 1A | 5 | 56 | 77 | 167 | 191 | 1.14 |
| 1A | 6 | 55 | 75 | 158 | 181 | 1.15 |
| 1B | 12 | 46 | 62 | 128 | 145 | 1.06* |

*20,000 rpm passes through the Magic Lab were used.

Table 11 shows the viscosity loss of each Sample. Viscosity loss was calculated comparing the viscosity after the six passes through the Magic Lab to the final viscosity after the filterability ratio test. Samples 1A, 1B, and 2 suffered less viscosity loss than Comparative Samples C1A, C1B, and C2A.

TABLE 11
Viscosity loss at 30 rpm after 6 passes through the
Magic Lab.

| Sample | Viscosity loss |
|---|---|
| 1A | 0% |
| 1B | 4%* |
| C1A | 76% |
| C1B | 14% |
| 2 | 3% |
| C2A | Plugged AP25 |

*measured at 30 rpm, after 12 passes at 20,000 rpm.

### Example II-8

### Differential Scanning Calorimetry (DSC) Rheology Characterization

Viscoelastic behavior was measured using a TA Instruments Q1000 Differential scanning calorimeter. Solid Samples at ambient condition were loaded on aluminum pans into the DSC. The instrument was equilibrated at 15.00° C. and data storage was set to “on” with a sampling interval of 1.00 sec/point. The temperature was ramped at 10.00° C./minute to 90° C. and the end of cycle 1 was marked. The initial heating to 90° C. minimized the contribution from the heat of vaporization on the final ramp up in temperature. An isothermal condition was maintained for 10.00 minutes. The temperature was then ramped at 10.00° C./minute to −50° C. The end of cycle 2 was marked. An isothermal condition was maintained for 2 minutes, then the temperature was ramped at 10.00° C./minute to 250.00° C. The end of cycle 3 was marked.

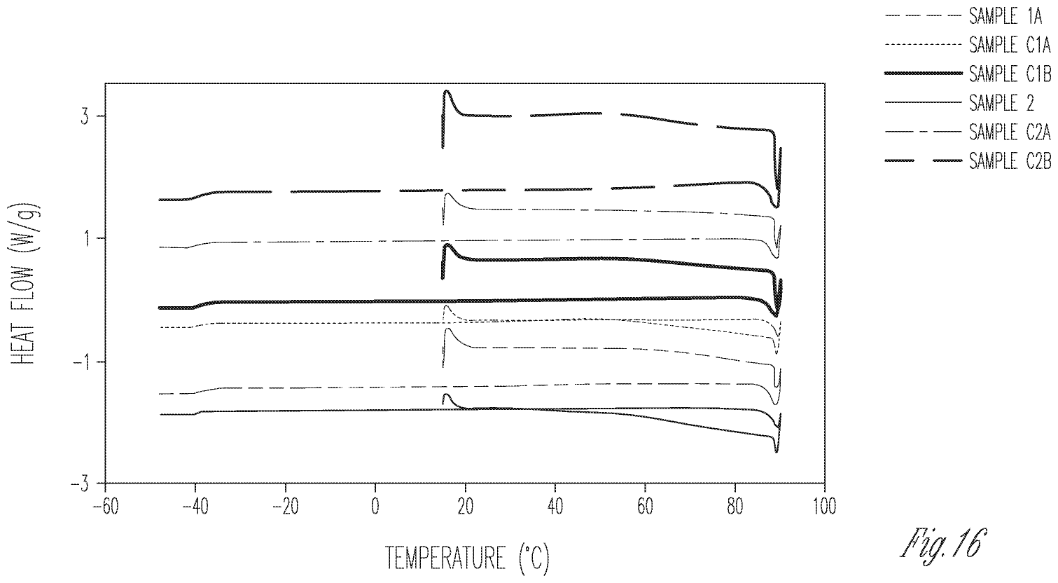

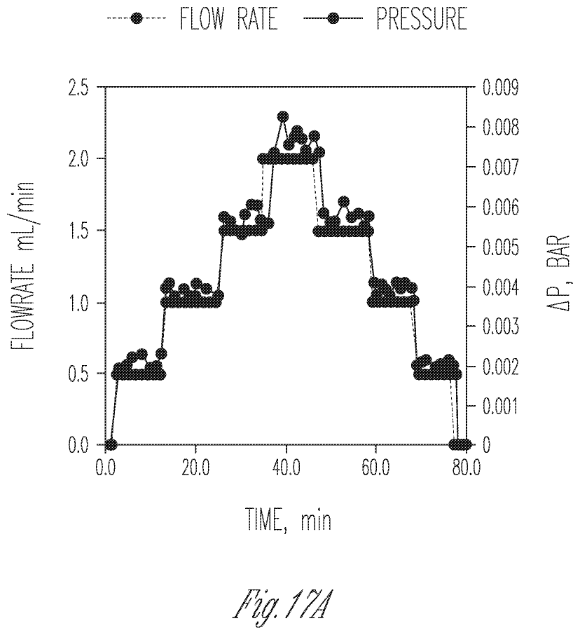

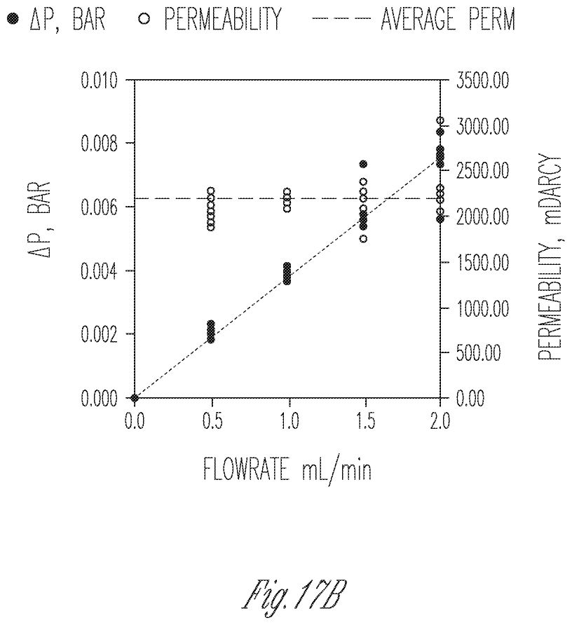

The results are shown in FIG. 16, which illustrates heat flow versus temperature during the scan to 90° C. with subsequent cooling to −50° C.

### Example II-9

### Inductively Coupled Plasma (ICP) Atomic Emission Spectroscopy Characterization

Solid Samples were prepared according to EPA 200.7 for analysis of ions in waste water. They were acid digested using a Milestone UltraWave and injected in aqueous form on an Arcos MV ICP analyzer. The results are shown in Table 12.

TABLE 12
ICP results in μg/g.

| Sample | Ca | Cu | Fe | K | Mg | Mn | Na | P | S | Zn |
|---|---|---|---|---|---|---|---|---|---|---|
| 1A | 3921 | 2.8 | 275 | <125 | 31 | 1.3 | 3116 | <125 | 148 | 2.3 |
| 1B | 945 | <1.5 | 178 | 82 | 9.2 | 0.5 | 279 | 287 | 218 | <1.5 |
| C1A | 1282 | 1.0 | 112 | 1669 | 686 | 2.6 | 6070 | 2110 | 1005 | 3.8 |
| C1B | 264 | <0.2 | 210 | 596 | 165 | 2.0 | 1294 | 738 | 371 | 1.9 |
| 6 | 8566 | 1.3 | 53 | 281 | 12860 | 18.7 | 247 | 11051 | 295 | 12.8 |
| C2A | 3753 | 7.1 | 11.5 | 1890 | 2625 | 5.6 | 5156 | 6489 | 2059 | 35.6 |
| C2B | 2342 | <0.2 | 69 | <125 | 387 | 13.0 | 362 | 2100 | 48 | 11.7 |

### Example II-10

### Ash and Total Nitrogen Characterization

Solid Samples were loaded into Shimadzu TOC-L/TNM-L for total nitrogen measurement. Solid Samples were loaded into a Thermolyne 30400 muffle furnace for ash measurement. The results are illustrated in Tables 13 and 14. Insufficient Sample 2 material was on-hand for total ash content testing.

TABLE 13
Total nitrogen content.

| Sample | μg/g |
|---|---|
| 1A | 2.7 |
| C1A | 15.5 |
| C1B | 2.4 |
| 2 | 6.1 |
| C2A | 13.3 |
| C2B | 3.6 |

TABLE 14
Total ash content.

| Sample | Ash (wt %) |
|---|---|
| 1A | 1.1 |
| 1B | 0.40 |
| C1A | 2.34 |
| C1B | 0.84 |
| C2A | 3.61 |
| C2B | 0.59 |

### Example II-11

### Sand Pack

Samples with a concentration of 1 g/L were prepared by slowing adding solid material to brine on a mixing plate with vigorous mixing. The brine was 35 g/L sea salt water. The Sample solutions were then placed in the Magic Lab described herein at Example II-7, with 6 passes at 26,000 rpm per pass. The solutions were filtered through a 12 μm filter. The viscosities were measured and recorded. The solutions were stabilized using 1000 ppm glutaraldehyde.

Samples were degassed using vacuum pump under stirring for 1 hr. This removed dissolvable gas bubbles that might come off during flooding causing measurement error. The samples were degassed by applying a vacuum to the stirred sample, with a cold trap between the sample and the vacuum.

To a dry Chromaflex® column by Kimble Chase having a length of 15 cm and a diameter of 2.5 cm, sand (US Silica™ Ottawa F-75 sand) was added and packed, with tapping and settling of sand performed with every one-half inch of sand added to the column. The column was fitted with an adjustable adapter to run any size pack length of interest. A pack length of 1 inch of sand was used in the 15 cm long sand-packed column.

The sand-packed column was connected to a water line in a feed water container, with the outlet connected to a vacuum via a trap. The vacuum line was run slowly allowing for −2 to −3 psi pressure and the water was allowed to climb slowly through the sand and displace air. The trap between the column and the vacuum was allowed to fill with 100 mL of solution before turning the vacuum line off. The difference between the weight of water lost from the water container and the weight of water in the trap corresponded to the weight of water occupying the pore space and the lines (dead volume). The difference was converted into pore volume using the density (total pore volume=4.8 mL), and then was converted to porosity using the total volume of the sand pack, wherein porosity %=(volume of space)/(volume of sand pack) (porosity=38%).

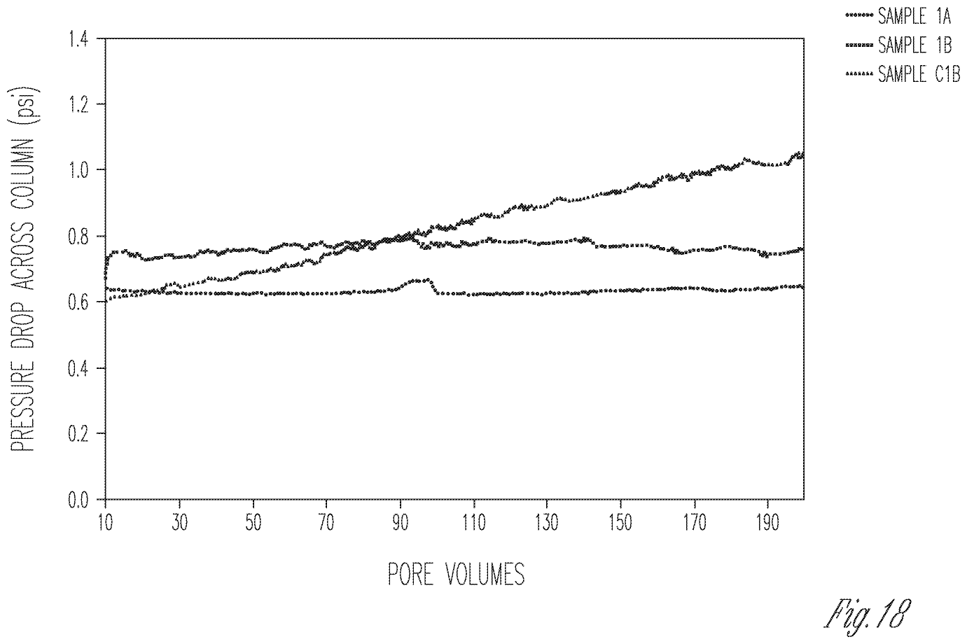

A peristaltic HPLC pump connected to an Additel 680 digital pressure gauge was connected to the inlet of the sand pack column. Water was injected into the sand pack with a flow of 2 mL/min. The water was flowed for 10 minutes to establish pressure drop and then the flow was stopped. The system was allowed to stand for 10 minutes to establish the zero flow rate. The pressure gauge was reset to zero and a flow rate of 0.5 to 2 mL/min was used, while recording the pressure versus flow rate. The permeability of the sand pack column was then calculated using Darcy's law at each flow rate, wherein permeability (m²)=(flow rate (m³/s)*viscosity (Pas)*length of the column (m))/(area (m²)*pressure change (Pa)). FIG. 17A illustrates flowrate and delta P versus time, and FIG. 17B illustrates delta P and permeability versus flowrate. The average permeability of the sand pack column was about 2200 mDarcy. Then another procedure was performed to measure pressure drop versus total pore volumes of the dispersed Sample flowed through the column. The procedure began with injecting sea salt water only (with 35 g/L of sea salt) at 1 mL/min (i.e., 0.208 pore volumes per minute) through a fresh sand column for each Sample, until the flow became stable. Then injection of the dispersed beta-glucan was initiated as the first pore volume was counted, which at first caused a slight pressure rise to about 0.5 psi to about 0.8 psi due to the increased viscosity but then stabilized. The flowrate of the dispersed beta-glucan through the column was 1 mL/min. Effective polymers maintained their pressure drop, while ineffective polymers caused an increase in the pressure drop over time which would eventually result in plugging of the column. FIG. 18 illustrates the pressure drop across the column (psi) versus the total pore volumes for Samples 1A, 1B, and C1B, using a flow rate of 1 mL/min. Table 15 illustrates the percent that the pressure drop increased over 200 total pore volumes of flow, with Sample C1A plugging a 12 micron prefilter between the HPLC pump and the column before the completion of testing.

TABLE 15
Sand Pack.

| | | Sandpack ΔP increase |
|---|---|---|
| Sample | 12 micron pre-filter | over 200 pore volumes |
| C1A | Plugged | N/A |
| C1B | Passed | 67.6% |
| 1A | Passed | 2.2% |
| 1B | Passed | 5.1% |

### Example II-12

### Oxalic Acid

Samples were prepared by adding 1 gram solid to about 80 mL of water in a 250 mL beaker with stirring to disperse. Ethylenediaminetetraacetic acid (EDTA, 0.06 g) was added, the pH was adjusted to 12 with sodium hydroxide solution and stirring was continued for 30 minutes. Successive filtration was performed with a 0.45 μm syringe filter (PTFE) and a 0.2 μm syringe filter (nylon).

Standards of calcium oxalate at 1 g/kg and 0.1 g/kg were prepared by adding 100 mg solid to 80 mL of water in a beaker and stirring. EDTA (0.6 g) was added, adjust the pH to 12 with sodium hydroxide solution and stir for 30 minutes. Using a pipette, 1 mL of the mother solution was added to a 100 mL volumetric flask, and the flask was filled to the mark with water. The solution was homogenized, then filtered with 0.45 μm PTFE syringe filter and 0.2 μm nylon syringe filter, directly into a vial.

Samples (injection volume of 20 μL) were injected into an HPLC with pre-column (Kj0-4282 Phenomenex®) and Aminex HPX 87 H 300 mm×7.8 mm column (BIORAD 125-4010). The analysis conditions included a flowrate of 0.6 mL/min, a column temperature of 55° C. and UV detector wavelength at 210 nm. The retention time of calcium oxalate was about 7.4 minutes. The result is given in mg (oxalic acid) per kilogram (ppm) of sample, derived by using the following calculation: (Aₛₐₘₚₗₑ×PE_{standard}×M_{oxalic acid})/(A_{standard}×PEₛₐₘₚₗₑ×M_{calcium oxalate}), where A is the area of the oxalic acid peak from the HPLC, PE is the weight in grams and M is the molar mass. The molar mass of oxalic acid is 90.03 g/mol. The molar mass of (monohydrated) calcium oxalate is 146.10 g/mol. Table 16 illustrates the reduced amount of oxalic acid in samples 1B compared to samples C1A and C1B. The oxalic acid concentration in Sample 1B ranged from 52 ppm to 377 ppm over 15 data points collected.

TABLE 16
Oxalic acid concentration.

| Sample | Oxalic acid (ppm) |
|---|---|
| C1A | 12237.51 |
| C1B | 2125.88 |
| 1B | 52-317 |

The terms and expressions that have been employed are used as terms of description and not of limitation, and there is no intention in the use of such terms and expressions of excluding any equivalents of the features shown and described or portions thereof, but it is recognized that various modifications are possible within the scope of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of the present invention.

### Exemplary Aspects.

The following exemplary aspects are provided, the numbering of which is not to be construed as designating levels of importance:

Aspect 1 provides a refined beta-glucan.

Aspect 2 provides the refined beta-glucan of Aspect 1, wherein the beta-glucan is an isolated beta-glucan.

Aspect 3 provides the refined beta-glucan of any one of Aspects 1-2, in the form of a power, a dispersion in a liquid, a solution in a liquid, or a combination thereof.

Aspect 4 provides the refined beta-glucan of any one of Aspects 1-3, wherein the beta-glucan is a 1,3 beta-glucan.

Aspect 5 provides the refined beta-glucan of any one of Aspects 1-4, wherein the beta-glucan is a 1,3-1,6 beta-D-glucan.

Aspect 6 provides the refined beta-glucan of any one of Aspects 1-5, wherein the beta-glucan is a 1,3-1,4 beta-D-glucan.

Aspect 7 provides the refined beta-glucan of any one of Aspects 1-6, wherein the beta-glucan is scleroglucan.

Aspect 8 provides the refined beta-glucan of any one of Aspects 1-7, wherein the beta-glucan is schizophyllan.

Aspect 9 provides the refined beta-glucan of any one of Aspects 1-8, wherein a dispersed mixture of the beta-glucan in water at a concentration of 1 mg/mL has an obscuration of less than or equal to about 0.7%.

Aspect 10 provides the refined beta-glucan of any one of Aspects 1-9, wherein a dispersed mixture of the beta-glucan in water at a concentration of 1 mg/mL has an obscuration of about 0.01% to about 0.6%.

Aspect 11 provides the refined beta-glucan of any one of Aspects 7 and 9-10, wherein a dispersed mixture of the beta-glucan in water at a concentration of 1 mg/mL has an obscuration of about 0.001% to about 0.5%.

Aspect 12 provides the refined beta-glucan of any one of Aspects 7 and 9-11, wherein a dispersed mixture of the beta-glucan in water at a concentration of 1 mg/mL has an obscuration of about 0.01% to about 0.35%.

Aspect 13 provides the refined beta-glucan of any one of Aspects 8-12, wherein a dispersed mixture of the beta-glucan in water at a concentration of 1 mg/mL has an obscuration of about 0.3% to about 0.7%.

Aspect 14 provides the refined beta-glucan of any one of Aspects 8-13, wherein a dispersed mixture of the beta-glucan in water at a concentration of 1 mg/mL has an obscuration of about 0.4% to about 0.5%.

Aspect 15 provides the refined beta-glucan of any one of Aspects 1-14, wherein a dispersed mixture of the beta-glucan in water at a concentration of 1 mg/mL experiences less than a 50% increase in pressure drop across a sand-packed column having a total pore volume equal to one Sand Column Void Space Volume during passage of 200 Sand Column Void Space Volumes of the dispersed mixture through the sand-packed column.

Aspect 16 provides the refined beta-glucan of any one of Aspects 1-15, wherein a dispersed mixture of the beta-glucan in water at a concentration of 1 mg/mL experiences an about 0.1% to about 50% increase in pressure drop across a sand-packed column having a total pore volume equal to one Sand Column Void Space Volume during passage of 200 Sand Column Void Space Volumes of the dispersed mixture through the sand-packed column.

Aspect 17 provides the refined beta-glucan of any one of Aspects 1-16, wherein a dispersed mixture of the beta-glucan in water at a concentration of 1 mg/mL experiences an about 1% to about 10% increase in pressure drop across a sand-packed column having a total pore volume equal to one Sand Column Void Space Volume during passage of 200 Sand Column Void Space Volumes of the dispersed mixture through the sand-packed column.

Aspect 18 provides the refined beta-glucan of any one of Aspects 1-17, wherein the beta-glucan has an oxalic acid concentration of about 5 ppm to about 1000 ppm.

Aspect 19 provides the refined beta-glucan of any one of Aspects 1-18, wherein the beta-glucan has an oxalic acid concentration of about 10 ppm to about 500 ppm.

Aspect 20 provides the refined beta-glucan of any one of Aspects 1-19, wherein the T_{g} of the beta-glucan as measured by onset of storage modulus change as detected by dynamic mechanical analysis is about 50° C. to about 90° C.

Aspect 21 provides the refined beta-glucan of any one of Aspects 1-20, wherein T_{g} of the beta-glucan as measured by onset of storage modulus change as detected by dynamic mechanical analysis is about 60° C. to about 80° C.

Aspect 22 provides the refined beta-glucan of any one of Aspects 7 and 9-21, wherein the T_{g} of the beta-glucan as measured by onset of storage modulus change as detected by dynamic mechanical analysis is about 70° C. to about 80° C.

Aspect 23 provides the refined beta-glucan of any one of Aspects 7 and 9-22, wherein the T_{g} of the beta-glucan as measured by onset of storage modulus change as detected by dynamic mechanical analysis is about 72° C. to about 76° C.

Aspect 24 provides the refined beta-glucan of any one of Aspects 8-23, wherein the T_{g} of the beta-glucan as measured by onset of storage modulus change as detected by dynamic mechanical analysis is about 60° C. to about 70° C.

Aspect 25 provides the refined beta-glucan of any one of Aspects 8-24, wherein the T_{g} of the beta-glucan as measured by onset of storage modulus change as detected by dynamic mechanical analysis is about 65° C. to about 66° C.

Aspect 26 provides the refined beta-glucan of any one of Aspects 1-25, wherein the T_{g} of the beta-glucan as measured by the peak tan delta as detected by dynamic mechanical analysis is about 70° C. to about 110° C.

Aspect 27 provides the refined beta-glucan of any one of Aspects 1-26, wherein the T_{g} of the beta-glucan as measured by the peak tan delta as detected by dynamic mechanical analysis is about 85° C. to about 100° C.

Aspect 28 provides the refined beta-glucan of any one of Aspects 7 and 9-27, wherein the T_{g} of the beta-glucan as measured by the peak tan delta as detected by dynamic mechanical analysis is about 90° C. to about 105° C.

Aspect 29 provides the refined beta-glucan of any one of Aspects 7 and 9-28, wherein the T_{g} of the beta-glucan as measured by the peak tan delta as detected by dynamic mechanical analysis is about 97° C. to about 99° C.

Aspect 30 provides the refined beta-glucan of any one of Aspects 8-29, wherein the T_{g} of the beta-glucan as measured by the peak tan delta as detected by dynamic mechanical analysis is about 85° C. to about 95° C.

Aspect 31 provides the refined beta-glucan of any one of Aspects 8-30, wherein the T_{g} of the beta-glucan as measured by the peak tan delta as detected by dynamic mechanical analysis is about 89° C. to about 90° C.

Aspect 32 provides the refined beta-glucan of any one of Aspects 1-31, wherein AFM images of the beta-glucan are substantially free of monolithic globular domains larger than about 4 microns.

Aspect 33 provides the refined beta-glucan of any one of Aspects 1-32, wherein AFM images of the beta-glucan are substantially free of monolithic globular domains larger than about 2 microns.

Aspect 34 provides the refined beta-glucan of any one of Aspects 7 and 9-33, wherein AFM images of the beta-glucan are substantially free of monolithic globular domains larger than about 1 micron.

Aspect 35 provides the refined beta-glucan of any one of Aspects 8-34, wherein AFM images of the beta-glucan are substantially free of monolithic globular domains larger than about 2 microns.

Aspect 36 provides the refined beta-glucan of any one of Aspects 1-35, wherein the beta-glucan has a majority decomposition temperature of about 300° C. to about 350° C.

Aspect 37 provides the refined beta-glucan of any one of Aspects 1-36, wherein the beta-glucan has a majority decomposition temperature of about 315° C. to about 340° C.

Aspect 38 provides the refined beta-glucan of any one of Aspects 7 and 9-37, wherein the beta-glucan has a majority decomposition temperature of about 330° C. to about 350° C.

Aspect 39 provides the refined beta-glucan of any one of Aspects 7 and 9-38, wherein the beta-glucan has a majority decomposition temperature of about 335° C. to about 345° C.

Aspect 40 provides the refined beta-glucan of any one of Aspects 8-39, wherein the beta-glucan has a majority decomposition temperature of about 340° C. to about 355° C.

Aspect 41 provides the refined beta-glucan of any one of Aspects 8-40, wherein the beta-glucan has a majority decomposition temperature of about 345° C. to about 350° C.

Aspect 42 provides the refined beta-glucan of any one of Aspects 1-41, wherein about 80 wt % to about 98 wt % of the beta-glucan is dry matter.

Aspect 43 provides the refined beta-glucan of any one of Aspects 1-42, wherein about 88 wt % to about 94.5 wt % of the beta-glucan is dry matter.

Aspect 44 provides the refined beta-glucan of any one of Aspects 1-43, wherein a solution of the beta-glucan in water prepared by subjecting to a shear of about 260,000 s⁻¹ or 200,000 s⁻¹ for about 0.01 s to about 2 s has a viscosity that is at least about 70% of an ultimate viscosity of the solution.

Aspect 45 provides the refined beta-glucan of any one of Aspects 1-44, wherein a solution of the beta-glucan in water prepared by subjecting to a shear of about 260,000 s⁻¹ or 200,000 s⁻¹ for about 0.01 s to about 2 s has a viscosity that is at least about 90% of an ultimate viscosity of the solution.

Aspect 46 provides the refined beta-glucan of any one of Aspects 44-45, wherein the ultimate viscosity of the solution is the viscosity of a solution of the beta-glucan in water prepared by subjecting to a shear of about 260,000 s⁻¹ for about 0.06 s to about 6 s, or at 200,000 s⁻¹ for about 0.12 s to about 12 s.

Aspect 47 provides the refined beta-glucan of any one of Aspects 1-46, wherein a 2 g/L solution of the beta-glucan in water prepared by subjecting the solution to a shear of about 260,000 s⁻¹ for about 0.06 s to about 6 s, or at 200,000 s⁻¹ for about 0.12 s to about 12 s, provides a sheared solution that has a Filterability Ratio that is about 1.01 to about 1.3.

Aspect 48 provides the refined beta-glucan of any one of Aspects 1-47, wherein a 2 g/L solution of the beta-glucan in water prepared by subjecting the solution to a shear of about 260,000 s⁻¹ for about 0.06 s to about 6 s, or at 200,000 s⁻¹ for about 0.12 s to about 12 s, provides a sheared solution that has a Filterability Ratio that is about 1.01 to about 1.25.

Aspect 49 provides the refined beta-glucan of any one of Aspects 7 and 9-48, wherein a 2 g/L solution of the beta-glucan in water prepared by subjecting the solution to a shear of about 260,000 s⁻¹ for about 0.06 s to about 6 s, or at 200,000 s⁻¹ for about 0.12 s to about 12 s, provides a sheared solution that has a Filterability Ratio that is about 1.01 to 1.2.

Aspect 50 provides the refined beta-glucan of any one of Aspects 8-49, wherein a 2 g/L solution of the beta-glucan in water prepared by subjecting the solution to a shear of about 260,000 s⁻¹ for about 0.06 s to about 6 s, or at 200,000 s⁻¹ for about 0.12 s to about 12 s, provides a sheared solution that has a Filterability Ratio that is about 1.15 to 1.25.

Aspect 51 provides the refined beta-glucan of any one of Aspects 1-50, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a filtered solution that has a viscosity that is about 90% to about 100% of the original viscosity.

Aspect 52 provides the refined beta-glucan of any one of Aspects 1-51, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a filtered solution that has a viscosity that is about 95% to about 100% of the original viscosity.

Aspect 53 provides the refined beta-glucan of any one of Aspects 7 and 9-52, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a filtered solution that has a viscosity that is about 98% to about 100% of the original viscosity.

Aspect 54 provides the refined beta-glucan of any one of Aspects 7 and 9-53, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a filtered solution that has a viscosity that is about 99.5% to about 100% of the original viscosity.

Aspect 55 provides the refined beta-glucan of any one of Aspects 8-54, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a sheared solution that has a viscosity that is about 94% to about 99% of the original viscosity.

Aspect 56 provides the refined beta-glucan of any one of Aspects 8-55, wherein a 2 g/L solution of the beta-glucan in water prepared by mixing at 260,000 s⁻¹ for about 0.06 s to about 6 s, or at 200,000 s⁻¹ for about 0.12 s to about 12 s, has an original viscosity, and subjecting the solution to filtration through a 1.2 micron filter provides a filtered solution that has a viscosity that is about 96% to about 98% of the original viscosity.

Aspect 57 provides the refined beta-glucan of any one of Aspects 1-56, wherein the beta-glucan has a total atomic calcium content of about 300 μg/g to about 10,000 μg/g.

Aspect 58 provides the refined beta-glucan of any one of Aspects 1-57, wherein the beta-glucan has a total atomic calcium content of about 500 μg/g to about 9,000 μg/g.

Aspect 59 provides the refined beta-glucan of any one of Aspects 7 and 9-58, wherein the beta-glucan has a total atomic calcium content of about 3,500 μg/g to about 4,500 μg/g.

Aspect 60 provides the refined beta-glucan of any one of Aspects 7 and 9-59, wherein the beta-glucan has a total atomic calcium content of about 3,800 μg/g to about 4,100 μg/g.

Aspect 61 provides the refined beta-glucan of any one of Aspects 8-60, wherein the beta-glucan has a total atomic calcium content of about 7,000 μg/g to about 10,000 μg/g.

Aspect 62 provides the refined beta-glucan of any one of Aspects 8-61, wherein the beta-glucan has a total atomic calcium content of about 8,000 μg/g to about 9,000 μg/g.

Aspect 63 provides the refined beta-glucan of any one of Aspects 1-62, wherein the beta-glucan has a total atomic copper content of about 0 μg/g to about 4 μg/g.

Aspect 64 provides the refined beta-glucan of any one of Aspects 1-63, wherein the beta-glucan has a total atomic copper content of about 0 μg/g to about 3 μg/g.

Aspect 65 provides the refined beta-glucan of any one of Aspects 7 and 9-64, wherein the beta-glucan has a total atomic copper content of about 0 μg/g to about 4 μg/g.

Aspect 66 provides the refined beta-glucan of any one of Aspects 7 and 9-65, wherein the beta-glucan has a total atomic copper content of about 0 μg/g to about 3.5 μg/g.

Aspect 67 provides the refined beta-glucan of any one of Aspects 8-66, wherein the beta-glucan has a total atomic copper content of about 0.5 μg/g to about 2 μg/g.

Aspect 68 provides the refined beta-glucan of any one of Aspects 8-67, wherein the beta-glucan has a total atomic copper content of about 1.1 μg/g to about 1.5 μg/g.

Aspect 69 provides the refined beta-glucan of any one of Aspects 1-68, wherein the beta-glucan has a total atomic iron content of about 10 μg/g to about 300 μg/g.

Aspect 70 provides the refined beta-glucan of any one of Aspects 1-69, wherein the beta-glucan has a total atomic iron content of about 40 μg/g to about 290 μg/g.

Aspect 71 provides the refined beta-glucan of any one of Aspects 7 and 9-70, wherein the beta-glucan has a total atomic iron content of about 150 μg/g to about 300 μg/g.

Aspect 72 provides the refined beta-glucan of any one of Aspects 7 and 9-71, wherein the beta-glucan has a total atomic iron content of about 160 μg/g to about 290 μg/g.

Aspect 73 provides the refined beta-glucan of any one of Aspects 8-72, wherein the beta-glucan has a total atomic iron content of about 30 μg/g to about 80 μg/g.

Aspect 74 provides the refined beta-glucan of any one of Aspects 8-73, wherein the beta-glucan has a total atomic iron content of about 45 μg/g to about 60 μg/g.

Aspect 75 provides the refined beta-glucan of any one of Aspects 1-74, wherein the beta-glucan has a total atomic potassium content of about 0 μg/g to about 500 μg/g.

Aspect 76 provides the refined beta-glucan of any one of Aspects 1-75, wherein the beta-glucan has a total atomic potassium content of about 0 μg/g to about 300 μg/g.

Aspect 77 provides the refined beta-glucan of any one of Aspects 7 and 9-76, wherein the beta-glucan has a total atomic potassium content of about 0 μg/g to about 200 μg/g.

Aspect 78 provides the refined beta-glucan of any one of Aspects 7 and 9-77, wherein the beta-glucan has a total atomic potassium content of about 0 μg/g to about 125 μg/g.

Aspect 79 provides the refined beta-glucan of any one of Aspects 8-78, wherein the beta-glucan has a total atomic potassium content of about 250 μg/g to about 310 μg/g.

Aspect 80 provides the refined beta-glucan of any one of Aspects 8-79, wherein the beta-glucan has a total atomic potassium content of about 260 μg/g to about 300 μg/g.

Aspect 81 provides the refined beta-glucan of any one of Aspects 1-80, wherein the beta-glucan has a total atomic magnesium content of about 1 μg/g to about 14,000 μg/g.

Aspect 82 provides the refined beta-glucan of any one of Aspects 1-81, wherein the beta-glucan has a total atomic magnesium content of about 5 μg/g to about 13,000 μg/g.

Aspect 83 provides the refined beta-glucan of any one of Aspects 7 and 9-82, wherein the beta-glucan has a total atomic magnesium content of about 1 μg/g to about 100 μg/g.

Aspect 84 provides the refined beta-glucan of any one of Aspects 7 and 9-83, wherein the beta-glucan has a total atomic magnesium content of about 5 μg/g to about 50 μg/g.

Aspect 85 provides the refined beta-glucan of any one of Aspects 8-84, wherein the beta-glucan has a total atomic magnesium content of about 12,000 μg/g to about 14,000 μg/g.

Aspect 86 provides the refined beta-glucan of any one of Aspects 8-85, wherein the beta-glucan has a total atomic magnesium content of about 12,800 μg/g to about 12,900 μg/g.

Aspect 87 provides the refined beta-glucan of any one of Aspects 1-86, wherein the beta-glucan has a total atomic manganese content of about 0.1 μg/g to about 30 μg/g.

Aspect 88 provides the refined beta-glucan of any one of Aspects 1-87, wherein the beta-glucan has a total atomic manganese content of about 0.2 μg/g to about 20 μg/g.

Aspect 89 provides the refined beta-glucan of any one of Aspects 7 and 9-88, wherein the beta-glucan has a total atomic manganese content of about 0.1 μg/g to about 2 μg/g.

Aspect 90 provides the refined beta-glucan of any one of Aspects 7 and 10-89, wherein the beta-glucan has a total atomic manganese content of about 0.2 μg/g to about 1.9 μg/g.

Aspect 91 provides the refined beta-glucan of any one of Aspects 8-90, wherein the beta-glucan has a total atomic manganese content of about 14 μg/g to about 25 μg/g.

Aspect 92 provides the refined beta-glucan of any one of Aspects 8-91, wherein the beta-glucan has a total atomic manganese content of about 16 μg/g to about 22 μg/g.

Aspect 93 provides the refined beta-glucan of any one of Aspects 1-92, wherein the beta-glucan has a total atomic sodium content of about 100 μg/g to about 4,000 μg/g.

Aspect 94 provides the refined beta-glucan of any one of Aspects 1-93, wherein the beta-glucan has a total atomic sodium content of about 200 μg/g to about 3,200 μg/g.

Aspect 95 provides the refined beta-glucan of any one of Aspects 7 and 9-94, wherein the beta-glucan has a total atomic sodium content of about 100 μg/g to about 3,500 μg/g.

Aspect 96 provides the refined beta-glucan of any one of Aspects 7 and 9-95, wherein the beta-glucan has a total atomic sodium content of about 250 μg/g to about 3,200 μg/g.

Aspect 97 provides the refined beta-glucan of any one of Aspects 8-96, wherein the beta-glucan has a total atomic sodium content of about 150 μg/g to about 350 μg/g.

Aspect 98 provides the refined beta-glucan of any one of Aspects 8-97, wherein the beta-glucan has a total atomic sodium content of about 200 μg/g to about 300 μg/g.

Aspect 99 provides the refined beta-glucan of any one of Aspects 1-98, wherein the beta-glucan has a total atomic phosphorus content of about 0 μg/g to about 15,000 μg/g.

Aspect 100 provides the refined beta-glucan of any one of Aspects 1-99, wherein the beta-glucan has a total atomic phosphorus content of about 0 μg/g to about 12,000 μg/g.

Aspect 101 provides the refined beta-glucan of any one of Aspects 7 and 9-100, wherein the beta-glucan has a total atomic phosphorus content of about 0 μg/g to about 500 μg/g.

Aspect 102 provides the refined beta-glucan of any one of Aspects 7 and 9-101, wherein the beta-glucan has a total atomic phosphorus content of about 0 μg/g to about 300 μg/g.

Aspect 103 provides the refined beta-glucan of any one of Aspects 8-102, wherein the beta-glucan has a total atomic phosphorus content of about 10,000 μg/g, to about 12,000 μg/g.

Aspect 104 provides the refined beta-glucan of any one of Aspects 8-103, wherein the beta-glucan has a total atomic phosphorus content of about 10,500 μg/g to about 11,500 μg/g.

Aspect 105 provides the refined beta-glucan of any one of Aspects 1-104, wherein the beta-glucan has a total atomic sulfur content of about 50 μg/g to about 400 μg/g.

Aspect 106 provides the refined beta-glucan of any one of Aspects 1-105, wherein the beta-glucan has a total atomic sulfur content of about 100 μg/g to about 350 μg/g.

Aspect 107 provides the refined beta-glucan of any one of Aspects 7 and 9-106, wherein the beta-glucan has a total atomic sulfur content of about 50 μg/g to about 300 μg/g.

Aspect 108 provides the refined beta-glucan of any one of Aspects 7 and 9-107, wherein the beta-glucan has a total atomic sulfur content of about 100 μg/g to about 250 μg/g.

Aspect 109 provides the refined beta-glucan of any one of Aspects 8-108, wherein the beta-glucan has a total atomic sulfur content of about 200 μg/g to about 400 μg/g.

Aspect 110 provides the refined beta-glucan of any one of Aspects 8-109, wherein the beta-glucan has a total atomic sulfur content of about 250 μg/g to about 350 μg/g.

Aspect 111 provides the refined beta-glucan of any one of Aspects 1-110, wherein the beta-glucan has a total atomic zinc content of about 0 μg/g, to about 15 μg/g.

Aspect 112 provides the refined beta-glucan of any one of Aspects 1-111, wherein the beta-glucan has a total atomic zinc content of about 0 μg/g to about 13 μg/g.

Aspect 113 provides the refined beta-glucan of any one of Aspects 7 and 9-112, wherein the beta-glucan has a total atomic zinc content of about 0 μg/g to about 4 μg/g.

Aspect 114 provides the refined beta-glucan of any one of Aspects 7 and 9-113, wherein the beta-glucan has a total atomic zinc content of about 0 μg/g to about 3 μg/g.

Aspect 115 provides the refined beta-glucan of any one of Aspects 8-114, wherein the beta-glucan has a total atomic zinc content of about 10 μg/g to about 16 μg/g.

Aspect 116 provides the refined beta-glucan of any one of Aspects 8-115, wherein the beta-glucan has a total atomic zinc content of about 12 μg/g to about 14 μg/g.

Aspect 117 provides the refined beta-glucan of any one of Aspects 1-116, wherein protein is about 0.01 wt % to about 2 wt % of the beta-glucan.

Aspect 118 provides the refined beta-glucan of any one of Aspects 1-117, wherein protein is about 0.10 wt % to about 0.45 wt % of the beta-glucan.

Aspect 119 provides the refined beta-glucan of any one of Aspects 7 and 9-118, wherein protein is about 0.05 wt % to about 0.3 wt % of the beta-glucan.

Aspect 120 provides the refined beta-glucan of any one of Aspects 7 and 9-119, wherein protein is about 0.10 wt % to about 0.20 wt % of the beta-glucan.

Aspect 121 provides the refined beta-glucan of any one of Aspects 8-120, wherein protein is about 0.2 wt % to about 0.6 wt % of the beta-glucan.

Aspect 122 provides the refined beta-glucan of any one of Aspects 8-121, wherein protein is about 0.35 wt % to about 0.45 wt % of the beta-glucan.

Aspect 123 provides the refined beta-glucan of any one of Aspects 1-122, wherein the beta-glucan has a total atomic nitrogen content of about 1 μg/g to about 10 μg/g.

Aspect 124 provides the refined beta-glucan of any one of Aspects 1-123, wherein the beta-glucan has a total atomic nitrogen content of about 2 μg/g to about 7 μg/g.

Aspect 125 provides the refined beta-glucan of any one of Aspects 7 and 9-124, wherein the beta-glucan has a total atomic nitrogen content of about 1 μg/g to about 5 μg/g.

Aspect 126 provides the refined beta-glucan of any one of Aspects 7 and 9-125, wherein the beta-glucan has a total atomic nitrogen content of about 2.5 μg/g to about 3 μg/g.

Aspect 127 provides the refined beta-glucan of any one of Aspects 8-126, wherein the beta-glucan has a total atomic nitrogen content of about 4 μg/g to about 8 μg/g.

Aspect 128 provides the refined beta-glucan of any one of Aspects 8-127, wherein the beta-glucan has a total atomic nitrogen content of about 5.5 μg/g, to about 6.5 μg/g.

Aspect 129 provides the refined beta-glucan of any one of Aspects 1-128, wherein upon total combustion the beta-glucan forms an ash that is about 0.01 wt % to about 3 wt % of the beta-glucan.

Aspect 130 provides the refined beta-glucan of any one of Aspects 1-129, wherein upon total combustion the beta-glucan forms an ash that is about 0.1 wt % to about 1.3 wt % of the beta-glucan.

Aspect 131 provides the refined beta-glucan of any one of Aspects 1-130, wherein upon total combustion the beta-glucan forms an ash that is about 0.01 wt % to about 0.5 wt % of the beta-glucan.

Aspect 132 provides a refined beta-glucan, wherein:

a dispersed mixture of the beta-glucan in water at a concentration of 1 mg/mL has an obscuration of less than or equal to about 0.7%,

the T_{g} of the beta-glucan as measured by onset of storage modulus change as detected by dynamic mechanical analysis is about 50° C. to about 90° C.,

the T_{g} of the beta-glucan as measured by the peak tan delta as detected by dynamic mechanical analysis is about 70° C. to about 110° C.,

the beta-glucan has a majority decomposition temperature of about 300° C. to about 350° C.,

about 80 wt % to about 98 wt % of the beta-glucan is dry matter,

the beta-glucan has a total atomic calcium content of about 300 μg/g to about 10,000 μg/g,

the beta-glucan has a total atomic copper content of about 0 μg/g to about 4 μg/g,

the beta-glucan has a total atomic iron content of about 10 μg/g to about 300 μg/g,

a total atomic potassium content of about 0 μg/g to about 500 μg/g,

the beta-glucan has a total atomic magnesium content of about 1 μg/g to about 14,000 μg/g,

the beta-glucan has a total atomic manganese content of about 0.1 μg/g to about 30 μg/g,

the beta-glucan has a total atomic sodium content f about 100 μg/g to about 4,000 μg/g,

the beta-glucan has a total atomic phosphorus content of about 0 μg/g to about 15,000 μg/g,

the beta-glucan has a total atomic sulfur content of about 50 μg/g to about 400 μg/g,

the beta-glucan has a total atomic zinc content of about 0 μg/g to about 15 μg/g, and

the beta-glucan has a total atomic nitrogen content of about 1 μg/g to about 10 μg/g.

Aspect 133 provides a refined beta-glucan, wherein:

a dispersed mixture of the beta-glucan in water at a concentration of 1 mg/mL has an obscuration of about 0.001% to about 0.6%,

the T_{g} of the beta-glucan as measured by onset of storage modulus change as detected by dynamic mechanical analysis is about 60° C. to about 80° C.,

the T_{g} of the beta-glucan as measured by the peak tan delta as detected by dynamic mechanical analysis is about 85° C. to about 100° C.,

the beta-glucan has a majority decomposition temperature of about 315° C. to about 340° C.,

about 80 wt % to about 98 wt % of the beta-glucan is dry matter,

the beta-glucan has a total atomic calcium content of about 500 μg/g to about 9,000 μg/g,

the beta-glucan has a total atomic copper content of about 0 μg/g to about 3 μg/g,

the beta-glucan has a total atomic iron content of about 40 μg/g to about 290 μg/g,

the beta-glucan has a total atomic potassium content of about 0 μg/g to about 300 μg/g,

the beta-glucan has a total atomic magnesium content of about 5 μg/g to about 13,000 μg/g,

the beta-glucan has a total atomic manganese content of about 1 μg/g to about 20 μg/g,

the beta-glucan has a total atomic sodium content of about 200 μg/g to about 3,200 μg/g,

the beta-glucan has a total atomic phosphorus content of about 0 μg/g to about 12,000 μg/g,

the beta-glucan has a total atomic sulfur content of about 100 μg/g to about 350 μg/g,

the beta-glucan has a total atomic zinc content of about 0 μg/g to about 13 μg/g, and

the beta-glucan has a total atomic nitrogen content of about 2 μg/g to about 7 μg/g.

Aspect 134 provides a refined beta-glucan, wherein:

the beta-glucan is scleroglucan,

a dispersed mixture of the beta-glucan in water at a concentration of 1 mg/mL has an obscuration of about 0.001% to about 0.5%,

the T_{g} of the beta-glucan as measured by onset of storage modulus change as detected by dynamic mechanical analysis is about 70° C. to about 80° C.,

the T_{g} of the beta-glucan as measured by the peak tan delta as detected by dynamic mechanical analysis is about 90° C. to about 105° C.,

the beta-glucan has a majority decomposition temperature of about 330° C. to about 350° C.,

about 80 wt % to about 98 wt % of the beta-glucan is dry matter,

the beta-glucan has a total atomic calcium content of about 300 μg/g to about 4,500 μg/g,

the beta-glucan has a total atomic copper content of about 0 μg/g to about 4 μg/g,

the beta-glucan has a total atomic iron content of about 150 μg/g to about 300 μg/g,

the beta-glucan has a total atomic potassium content of about 0 μg/g to about 200 μg/g,

the beta-glucan has a total atomic magnesium content of about 1 μg/g to about 100 μg/g,

the beta-glucan has a total atomic manganese content of about 0.2 μg/g to about 2 μg/g,

the beta-glucan has a total atomic sodium content of about 100 μg/g to about 3,500 μg/g,

the beta-glucan has a total atomic phosphorus is content of about 0 μg/g to about 500 μg/g,

the beta-glucan has a total atomic sulfur content of about 50 μg/g to about 300 μg/g,

the beta-glucan has a total atomic zinc content of about 0 μg/g to about 4 μg/g,

the beta-glucan has a total atomic nitrogen content of about 1 μg/g to about 5 μg/g, and

upon total combustion the beta-glucan forms an ash that is about 0.1 wt % to about 1.3 wt % of the beta-glucan.

Aspect 135 provides a refined beta-glucan, wherein:

the beta-glucan is scleroglucan,

protein is about 0.10 wt % to about 0.20 wt % of the beta-glucan,

a dispersed mixture of the beta-glucan in water at a concentration of 1 mg/mL has an obscuration of about 0.01% to about 0.35%,

the T_{g} of the beta-glucan as measured by onset of storage modulus change as detected by dynamic mechanical analysis is about 72° C. to about 76° C.,

the T_{g} of the beta-glucan as measured by the peak tan delta as detected by dynamic mechanical analysis is about 97° C. to about 99° C.,

the beta-glucan has a majority decomposition temperature of about 335° C. to about 345° C.,

about 80 wt % to about 98 wt % of the beta-glucan is dry matter,

the beta-glucan has a total atomic calcium content of about 500 μg/g to about 4,100 μg/g,

the beta-glucan has a total atomic copper content of about 0 μg/g to about 3.5 μg/g,

the beta-glucan has a total atomic iron content of about 60 μg/g to about 290 μg/g,

the beta-glucan has a total atomic potassium content of about 0 μg/g to about 125 μg/g,

the beta-glucan has a total atomic magnesium content of about 5 μg/g to about 50 μg/g,

the beta-glucan has a total atomic manganese content of about 0.2 μg/g to about 1.9 μg/g,

the beta-glucan has a total atomic sodium content of about 250 μg/g to about 3,200 μg/g,

the beta-glucan has a total atomic phosphorus is content of about 0 μg/g to about 300 μg/g,

the beta-glucan has a total atomic sulfur content of about 100 μg/g to about 250 μg/g,

the beta-glucan has a total atomic zinc content of about 0 μg/g to about 3 μg/g,

the beta-glucan has a total atomic nitrogen content of about 2.5 μg/g to about 3 μg/g, and

upon total combustion the beta-glucan forms an ash that is about 0.1 wt % to about 1.2 wt % of the beta-glucan.

Aspect 136 provides a refined beta-glucan, wherein:

the beta-glucan is schizophyllan,

a dispersed mixture of the beta-glucan in water at a concentration of 1 mg/mL has an obscuration of about 0.3% to about 0.7%,

the T_{g} of the beta-glucan as measured by onset of storage modulus change as detected by dynamic mechanical analysis is about 60° C. to about 70° C.,

the T_{g} of the beta-glucan as measured by the peak tan delta as detected by dynamic mechanical analysis is about 85° C. to about 95° C.,

the beta-glucan has a majority decomposition temperature of about 340° C. to about 355° C.,

about 80 wt % to about 98 wt % of the beta-glucan is dry matter,

the beta-glucan has a total atomic calcium content of about 7,000 μg/g to about 10,000 μg/g,

the beta-glucan has a total atomic copper content of about 0.5 μg/g to about 2 μg/g,

the beta-glucan has a total atomic iron content of about 30 μg/g to about 80 μg/g,

the beta-glucan has a total atomic potassium content of about 250 μg/g to about 310 μg/g,

the beta-glucan has a total atomic magnesium content of about 12,000 μg/g to about 14,000 μg/g,

the beta-glucan has a total atomic manganese content of about 14 μg/g to about 25 μg/g,

the beta-glucan has a total atomic sodium content of about 150 μg/g to about 350 μg/g,

the beta-glucan has a total atomic phosphorus content of about 10,000 μg/g to about 12,000 μg/g,

the beta-glucan has a total atomic sulfur content of about 200 μg/g to about 400 μg/g,

the beta-glucan has a total atomic zinc content of about 10 μg/g to about 16 μg/g, and

the beta-glucan has a total atomic nitrogen content of about 4 μg/g to about 8 μg/g.

Aspect 137 provides a refined beta-glucan, wherein:

the beta-glucan is schizophyllan,

protein is about 0.35 wt % to about 0.45 wt % of the beta-glucan,

a dispersed mixture of the beta-glucan in water at a concentration of 1 mg/mL has an obscuration of about 0.4% to about 0.5%,

the T_{g} of the beta-glucan as measured by onset of storage modulus change as detected by dynamic mechanical analysis is about 65° C. to about 66° C.,

the T_{g} of the beta-glucan as measured by the peak tan delta as detected by dynamic mechanical analysis is about 89° C. to about 90° C.,

the beta-glucan has a majority decomposition temperature of about 345° C. to about 350° C.,

about 80 wt % to about 98 wt % of the beta-glucan is dry matter,

the beta-glucan has a total atomic calcium content of about 8,000 μg/g to about 9,000 μg/g,

the beta-glucan has a total atomic copper content of about 1.1 μg/g to about 1.5 μg/g,

the beta-glucan has a total atomic iron content of about 45 μg/g to about 60 μg/g,

the beta-glucan has a total atomic potassium content of about 260 μg/g to about 300 μg/g,

the beta-glucan has a total atomic magnesium content of about 12,800 μg/g to about 12,900 μg/g,

the beta-glucan has a total atomic manganese content of about 16 μg/g to about 22 μg/g,

the beta-glucan has a total atomic sodium content of about 200 μg/g to about 300 μg/g,

the beta-glucan has a total atomic phosphorus content of about 10,500 μg/g to about 11,500 μg/g,

the beta-glucan has a total atomic sulfur content of about 250 μg/g to about 350 μg/g,

the beta-glucan has a total atomic zinc content of about 12 μg/g to about 14 μg/g, and

the beta-glucan has a total atomic nitrogen content of about 5.5 μg/g to about 6.5 μg/g.

Aspect 138 provides a composition comprising the refined beta-glucan of any one of Aspects 1-137.

Aspect 139 provides the composition of Aspect 138, wherein the composition is a solid, a liquid, a solution, or a combination thereof.

Aspect 140 provides the composition of any one of Aspects 138-139, wherein the composition is a liquid.

Aspect 141 provides the liquid of Aspect 140, wherein the liquid is an aqueous liquid.

Aspect 142 provides the liquid of any one of Aspects 140-141, wherein the beta-glucan is about 0.001 wt % to about 99.999 wt % of the liquid.

Aspect 143 provides the liquid of any one of Aspects 140-142, wherein the liquid is a liquid for treatment of a subterranean formation.

Aspect 144 provides the liquid of any one of Aspects 140-143, wherein the liquid is a liquid for enhanced oil recovery polymer flooding, for hydraulic fracturing, or a combination thereof.

Aspect 145 provides the composition of any one of Aspects 138-139, wherein the composition is a solid.

Aspect 146 provides the solid of Aspect 145, wherein the solid is a power.

Aspect 147 provides the solid of any one of Aspects 145-146, wherein the beta-glucan is about 0.001 wt % to about 99.999 wt % of the solid.

Aspect 148 provides a method of forming the refined beta-glucan of any one of Aspects 1-137, the method comprising:

filtering a solution of a crude beta-glucan, to form a filtrate comprising the refined beta-glucan of any one of Aspects 1-137.

Aspect 149 provides the method of Aspect 148, further comprising homogenizing the crude beta-glucan in water to form the solution of the crude beta-glucan.

Aspect 150 provides the method of Aspect 149, wherein the homogenizing occurs at about 40° C. to about 90° C.

Aspect 151 provides the method of any one of Aspects 148-150, wherein the solution has a pH of about 4 to about 7.

Aspect 152 provides the method of any one of Aspects 148-151, wherein the solution has a pH of about 5 to about 6.

Aspect 153 provides the method of any one of Aspects 148-152, further comprising acidifying the solution of the crude beta-glucan to precipitate oxalic acid therefrom, then raising the pH to about 4 to about 7 prior to the filtering.

Aspect 154 provides the method of Aspect 153, wherein the acidifying comprises adding acid to decrease the pH of the solution to about 1 to about 4.5.

Aspect 155 provides the method of any one of Aspects 153-154, wherein the acidifying comprises adding acid to decrease the pH of the solution to about 1.5 to about 3.5.

Aspect 156 provides the method of any one of Aspects 148-155, wherein the filtering comprises filtering through a filter.

Aspect 157 provides the method of any one of Aspects 148-156, wherein the filtering comprises adding one or more filter aids to the solution and filtering the solution through a filter.

Aspect 158 provides the method of Aspect 157, wherein the concentration of each filter aid in the solution is independently about 1 g/L to about 100 g/L.

Aspect 159 provides the method of any one of Aspects 157-158, wherein the concentration of each filter aid in the solution is independently about 2 g/L to about 50 g/L.

Aspect 160 provides the method of any one of Aspects 157-159, wherein the filter aid has a permeability of about 0.001 Darcy to about 30 Darcy.

Aspect 161 provides the method of any one of Aspects 157-160, wherein the filter aid has a permeability of about 1 Darcy to about 30 Darcy.

Aspect 162 provides the method of any one of Aspects 157-161, wherein the filter aid has a permeability of about 1.5 Darcy to about 5 Darcy.

Aspect 163 provides the method of any one of Aspects 157-162, wherein the filter aid has a permeability of about 0.001 Darcy to about 1 Darcy.

Aspect 164 provides the method of any one of Aspects 157-163, wherein the filter aid has a permeability of about 0.02 Darcy to about 0.200 Darcy.

Aspect 165 provides the method of any one of Aspects 157-164, wherein the one or more filter aids comprise a filter aid having a permeability of about 1 Darcy to about 30 Darcy and another filter aid having a permeability of about 0.001 Darcy to about 1 Darcy.

Aspect 166 provides the method of any one of Aspects 157-165, wherein the filtering comprises filtering all or a portion of the solution through the filter to form a filter cake on the filter, and filtering all of the solution through the filter cake on the filter.

Aspect 167 provides the method of any one of Aspects 157-166, wherein the filtering comprises filtering all or a portion of the solution through the filter to form a filer cake on the filter, adding additional filter aid to the filtrate, filtering all or a portion of the solution with the additional aid through the filter cake to form a second filter cake, and filtering all of the solution through the filter cake on the filter.

Aspect 168 provides the method of any one of Aspects 148-167, comprising performing the filtering at a temperature of about 40° C. to about 90° C.

Aspect 169 provides the method of any one of Aspects 148-168, comprising performing the filtering at a temperature of about 75° C. to about 85° C.

Aspect 170 provides the method of any one of Aspects 148-169, further comprising performing multiple cycles of the filtration.

Aspect 171 provides the method of any one of Aspects 148-170, further comprising precipitating biopolymer from the filtrate.

Aspect 172 provides the method of Aspect 171, wherein the precipitating comprises adding an organic solvent to the filtrate to decrease the solubility of the biopolymer therein, and draining liquid from the precipitated biopolymer.

Aspect 173 provides the method of Aspect 172, further comprising washing the precipitated biopolymer with an organic solvent and draining the organic solvent wash from the precipitated biopolymer.

Aspect 174 provides the method of any one of Aspects 171-173, further comprising drying the precipitated biopolymer.

Aspect 175 provides the method of Aspect 174, wherein the drying comprises drying such that the biopolymer has a dry matter content of about 80 wt % to about 98 wt %.

Aspect 176 provides the method of any one of Aspects 174-175, wherein the drying comprises drying to a dry matter content of about 85 wt % to about 95 wt %.

Aspect 177 provides the method of any one of Aspects 174-176, further comprising grinding the precipitated biopolymer, to provide the beta-glucan of any one of Aspects 1-137.

Aspect 178 provides the method of Aspect 177, wherein the grinding comprises grinding to a particle size of about 1000 microns or less.

Aspect 179 provides the method of any one of Aspects 177-178, wherein the winding comprises grinding to a particle size of about 500 microns or less.

Aspect 180 provides the method of any one of Aspects 177-179, wherein the grinding comprises grinding to a particle size of about 250 microns or less.

Aspect 181 provides a refined beta-glucan made by the method of any one of Aspects 148-180.

Aspect 182 provides a method of forming the refined beta-glucan of any one of Aspects 1-137, the method comprising:

filtering a solution of a crude beta-glucan, comprising adding one or more filter aids to the solution and filtering all or a portion of the solution through a filter to form a filter cake on a filter, and filtering all of the solution through the filter cake on the filter, to form a first filtrate;

filtering the first filtrate, comprising adding one or more filter aids to the solution and filtering all or a portion of the solution through a filter to form a filter cake on a filter, and filtering all of the solution through the filter cake on the filter, to form a second filtrate;

filtering the second filtrate, comprising adding one or more filter aids to the solution and filtering all or a portion of the solution through a filter to form a filter cake on a filter, and filtering all of the solution through the filter cake on the filter, to form a third filtrate;

precipitating biopolymer from the third filtrate, comprising adding an organic solvent to the filtrate to decrease the solubility of the biopolymer therein, and draining liquid from the precipitated biopolymer;

washing the biopolymer with an organic solvent and draining the organic solvent wash from the biopolymer;

drying the biopolymer such that the biopolymer has a dry matter content of about 80 wt % to about 98 wt %;

grinding the dried biopolymer to a particle size of equal to or less than about 1,000 microns, to provide the refined beta-glucan of any one of Aspects 1-137;

wherein
each filtration is independently performed at a temperature of about 40° C. to about 90° C.
the concentration of each filter aid is independently about 1 g/L to about 100 g/L, and
each filter aid independently has a permeability of about 0.001 Darcy to about 30 Darcy.

Aspect 183 provides a method of treating a subterranean formation, the method comprising:

placing the refined beta-glucan of any one of Aspects 1-137 in the subterranean formation.

Aspect 184 provides the method of Aspect 183, comprising performing a hydraulic fracturing operating in the subterranean formation using a liquid comprising the beta-glucan.

Aspect 185 provides the method of any one of Aspects 183-184, comprising performing an enhanced oil recovery procedure in the subterranean formation using a liquid comprising the beta-glucan.

Aspect 186 provides the method of Aspect 185, wherein the enhanced oil recovery procedure comprises polymer flooding.

Aspect 1875 provides the method of any one of Aspects 185-186, wherein the liquid comprising the beta-glucan in the subterranean formation sweeps petroleum in the subterranean formation toward a well.

Aspect 188 provides the method of Aspect 187, further comprising removing the petroleum from the well.

Aspect 189 provides the use of the refined beta-glucan of any one of Aspects 1-137 for treatment of a subterranean formation.

Aspect 190 provides the refined beta-glucan of any one of Aspects 1-130, having a bulk density of about 0.2 to about 0.6 kg/L.

Aspect 191 provides the refined beta-glucan of any one of Aspects 1-130, having a bulk density of about 0.3 to about 0.5 kg/L.

Aspect 192 provides the refined beta-glucan of any one of Aspects 1-130, wherein the beta-glucan has a particle size of about 0.01 microns to about 5,000 microns.

Aspect 193 provides the refined beta-glucan of any one of Aspects 1-130, wherein a majority of particles of the beta-glucan have a particle size of about 1.5 micron to about 500 microns and of about 700 micron to about 5,000 microns.

Aspect 194 provides the refined beta-glucan of any one of Aspects 1-130, wherein the beta-glucan is substantially free of particles having a particle size of greater than about 500 microns to less than about 700 microns, particles having a particle size greater than about 5,000 microns, and particles having a particle size of 0.01 microns to less than about 1.5 microns.

Aspect 195 provides the refined beta-glucan of any one of Aspects 1-130, wherein a majority of particles of the beta-glucan have a particle size of about 0.01 micron to about 0.8 microns and of about 1.05 micron to about 2,000 microns.

Aspect 196 provides the refined beta-glucan of any one of Aspects 1-130, wherein the beta-glucan is substantially free of particles having a particle size of greater than about 0.8 microns to less than about 1.05 microns and particles having a particle size greater than about 2,000 microns.

Aspect 197 provides the refined beta-glucan of any one of Aspects 1-130, wherein the beta-glucan has a purity of about 75 wt % to about 100 wt %.

Aspect 198 provides the refined beta-glucan of any one of Aspects 1-130, wherein the beta-glucan has a purity of about 82 wt % to about 92 wt %.

Aspect 199 provides the refined beta-glucan of any one of Aspects 15-17, wherein the sand-packed column has a permeability of about 0.001 Darcy to about 30 Darcy.

Aspect 200 provides the refined beta-glucan of any one of Aspects 15-17, wherein the sand-packed column has a permeability of about 1 Darcy to about 4 Darcy.

Aspect 201 provides the refined beta-glucan of any one of Aspects 15-17, wherein the dispersed mixture of the beta-glucan in water is a dispersed mixture of the beta-glucan in salt water.

Aspect 202 provides the refined beta-glucan of Aspect 201, wherein the salt water has a total dissolved solids level of about 1,000 mg/L to about 250,000 mg/L.

Aspect 203 provides the refined beta-glucan of any one of Aspects 201-202, wherein the salt water has a total dissolved solids level of about 20,000 mg/L to about 50,000 mg/L.

Aspect 204 provides the refined beta-glucan of any one of Aspects 15-17, wherein the flow rate of the dispersed mixture of the beta-glucan in water through the sand-packed column is about 0.01 to about 10 Sand Column Void Space Volumes/min.

Aspect 205 provides the refined beta-glucan of any one of Aspects 15-17, wherein the flow rate of the dispersed mixture of the beta-glucan in water through the sand-packed column is about 0.1 to about 0,3 Sand Column Void Space Volumes/min.

Aspect 206 provides the refined beta-glucan, composition, method, or use of any one or any combination of Aspects 1-205 optionally configured such that all elements or options recited are available to use or select from.

1. A refined beta-glucan, wherein a dispersed mixture of the beta-glucan in water at a concentration of 1 mg/mL experiences less than or equal to a 50% increase in pressure drop across a sand-packed column having a total pore volume equal to one Sand Column Void Space Volume during passage of 200 Sand Column Void Space Volumes of the dispersed mixture through the sand-packed column.

2. The refined beta-glucan of claim 1, wherein a dispersed mixture of the beta-glucan in salt water having a total dissolved solids level of about 20,000 mg/L to about 50,000 mg/L at a concentration of 1 mg/mL experiences an about 1% to about 10% increase in pressure drop across a sand-packed column having a permeability of about 1 Darcy to about 4 Darcy and having a total pore volume equal to one Sand Column Void Space Volume during passage of 200 Sand Column Void Space Volumes of the dispersed mixture through the sand-packed column at a flowrate of about 0.1 to about 0.3 Sand Column Void Space Volumes/min.

3. (canceled)

4. The refined beta-glucan of claim 1, wherein the beta-glucan has an oxalic acid concentration of about 5 ppm to about 1000 ppm.

5. (canceled)

6. The refined beta-glucan of claim 1, wherein the T_{g} of the beta-glucan as measured by the peak tan delta as detected by dynamic mechanical analysis is about 70° C. to about 110° C.

7. The refined beta-glucan of claim 1, wherein AFM images of the beta-glucan are substantially free of monolithic globular domains larger than about 4 microns.

8. (canceled)

9. The refined beta-glucan of claim 1, wherein about 80 wt % to about 98 wt % of the beta-glucan is dry matter.

10. The refined beta-glucan of claim 1, wherein the beta-glucan has a total atomic calcium content of about 300 μg/g to about 10,000 μg/g.

11. The refined beta-glucan of claim 1, wherein the beta-glucan has a total atomic iron content of about 10 μg/g to about 300 μg/g.

12. The refined beta-glucan of claim 1, wherein the beta-glucan has a total atomic potassium content of about 0 μg/g to about 500 μg/g.

13. The refined beta-glucan of claim 1, wherein the beta-glucan has a total atomic sodium content of about 100 μg/g to about 4,000 μg/g

14. (canceled)

15. The refined beta-glucan of claim 1, wherein protein is about 0.01 wt % to about 2 wt % of the beta-glucan.

16. (canceled)

17. The refined beta-glucan of claim 1, wherein upon total combustion the beta-glucan forms an ash that is about 0.01 wt % to about 3 wt % of the beta-glucan.

18. A method of treating a subterranean formation, the method comprising: placing the refined beta-glucan of claim 1 in the subterranean formation.

19. A refined beta-glucan, wherein:
a dispersed mixture of the beta-glucan in water at a concentration of 1 mg/mL has an obscuration of less than or equal to about 0.7%,
the T_{g} of the beta-glucan as measured by onset of storage modulus change as detected by dynamic mechanical analysis is about 50° C. to about 90° C.,
the T_{g} of the beta-glucan as measured by the peak tan delta as detected by dynamic mechanical analysis is about 70° C. to about 110° C.,
the beta-glucan has a majority decomposition temperature of about 300° C. to about 350° C.,
about 80 wt % to about 98 wt % of the beta-glucan is dry matter,
the beta-glucan has a total atomic calcium content of about 300 μg/g to about 10,000 μg/g,
the beta-glucan has a total atomic copper content of about 0 μg/g to about 4 μg/g,
the beta-glucan has a total atomic iron content of about 10 μg/g to about 300 μg/g,
a total atomic potassium content of about 0 μg/g to about 500 μg/g,
the beta-glucan has a total atomic magnesium content of about 1 μg/g to about 14,000 μg/g,
the beta-glucan has a total atomic manganese content of about 0.1 μg/g to about 30 μg/g,
the beta-glucan has a total atomic sodium content of about 100 μg/g to about 4,000 μg/g,
the beta-glucan has a total atomic phosphorus content of about 0 μg/g to about 15,000 μg/g,
the beta-glucan has a total atomic sulfur content of about 50 μg/g to about 400 μg/g,
the beta-glucan has a total atomic zinc content of about 0 μg/g to about 15 μg/g, and
the beta-glucan has a total atomic nitrogen content of about 1 μg/g to about 10 μg/g.

20. A method of forming a refined beta-glucan, the method comprising:
filtering a solution of a crude beta-glucan, to form a filtrate comprising the refined beta-glucan, wherein a dispersed mixture of the beta-glucan in water at a concentration of 1 mg/mL experiences less than or equal to a 50% increase in pressure drop across a sand-packed column having a total pore volume equal to one Sand Column Void Space Volume during passage of 200 Sand Column Void Space Volumes of the dispersed mixture through the sand-packed column.
